(19) United States
(12) Patent Application Publication
(10) Pub. No.: US 2023/0321105 A1
(43) Pub. Date: Oct. 12, 2023

(54) CELL SIGNAL DETECTION AND MODULATION IN DISEASE

(71) Applicant: Washington University, St. Louis, MO (US)

(72) Inventors: Stephanie Markovina, St. Louis, MO (US); Julie Schwarz, St. Louis, MO (US)

(73) Assignee: Washington University, St. Louis, MO (US)

(21) Appl. No.: 18/298,256

(22) Filed: Apr. 10, 2023

Related U.S. Application Data
(60) Provisional application No. 63/328,758, filed on Apr. 8, 2022.

(51) Int. Cl. A61K 31/519; G01N 33/574; A61P 35/00

(52) U.S. Cl. CPC A61K 31/519; G01N 33/574; A61P 35/00

(57) ABSTRACT

Among the various aspects of the present disclosure is the provision of compositions and methods to detect immune system activation and inhibition, including but not limited to a pro-tumor radioresistant tumor microenvironment, including but not limited to overexpressed SERPINB3, and the modulation of this microenvironment with modulators, including but not limited to STAT inhibitors, which can improve radiotherapy in cancer patients.

### CROSS-REFERENCE TO RELATED APPLICATIONS

This application claims priority from U.S. Provisional Application Ser. No. 63/328,758 filed on Apr. 8, 2022, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention entitled ‘020110_SEQ_LISTING_SUBSTITUTE.XML’, created on 6/20/2023, and sized at 25,596 bytes. The subject matter of the Sequence Listing is incorporated herein by reference in its entirety.

### FIELD OF THE INVENTION

The present disclosure generally relates to the detection and modulation of a pro-tumor, immunosuppressive microenvironment associated with radioresistance by the detection of SERPIN3B and the modulation of STAT.

### BACKGROUND OF THE INVENTION

Radiotherapy (RT) is commonly used in the treatment of patients with squamous cell carcinomas, including head and neck, esophageal, lung, and cervical cancer. RT can have both immunostimulatory and immunosuppressive effects, which in part influence the prognosis of cancer. The activation and infiltration of cytotoxic T cells post-radiation are critical to the curative activity of RT. However, tumors with an immunosuppressive tumor microenvironment (TME), dominated by myeloid cells tend to diminish T cell activity and may be more susceptible to the suppressive immune response induced by RT. These immune responses include increased M2 macrophage polarization, myeloid-derived suppressor cell (MDSC) infiltration, T cell inhibitory receptor expression as well as chemokine release upon radiation that alter TME. Chemokines are a subclass of cytokines with chemotactic properties that control the migration of cells and influence the composition of the tumor immune microenvironment. Some chemokines promote an immunostimulatory environment, such as CXCL9, CXCL10, CXCL11, and CXCL16, which improve dendritic cell activation and T cell trafficking to tumors. Conversely, CCL2, CCL5, CXCL1, CXCL8, and CXCL12 can be induced by RT and have the opposite effect of recruiting suppressive immune cells and inhibiting effector T cells, which are often correlated with poor treatment outcomes.

Squamous cell carcinoma antigen 1 (SCCA), encoded by the SERPINB3 gene locus and now known as SERPINB3, is a highly conserved cysteine proteinase inhibitor that interacts with lysosomal proteases upon lysosomal leakage and prevents cell death. We have recently demonstrated that SERPINB3 also protected cervix tumor cells against RT-induced cell death by preventing lysoptosis. In many cancers, SERPINB3/SCCA1 was highly expressed in tumors or in the circulation of cancer patients, including cervical, head and neck, lung, breast, and esophageal cancers, often associated with poor prognosis, treatment outcomes, and recurrence. In addition, elevated SERPINB3 expression was also found in autoimmune disorders and implicated in the induction of inflammatory cytokines. However, in both tumors and autoimmune diseases, the mechanistic link between SERPINB3 and immune regulation remains poorly understood.

Considering the increasing number of studies reporting the association of SERPINB3 with tumorigenesis, metastasis, prognosis, and recurrence, additional roles of SERPINB3, independent of proteinase-inhibitory activity, in tumor progression and resistance to therapy are likely.

### SUMMARY OF THE INVENTION

Among the various aspects of the present disclosure is the provision of compositions and methods to detect immune system activation and inhibition, including but not limited to the detection of overexpressed SERPINB3 that is indicative of a pro-tumor radioresistant tumor microenvironment. The present disclosure further provides compositions and methods for the modulation of the pro-tumor radioresistant tumor microenvironment with modulators, including but not limited to STAT inhibitors, to improve radiotherapy outcomes in cancer patients.

Squamous cell carcinoma antigen (SERPINB3) is highly expressed in many epithelial cancers and associated with poor prognosis, including radiation therapy resistance by an unknown mechanism. Using human cancer cells and PBMCs, it is shown herein using RNA sequencing that SERPINB3 upregulated CXCL1/8 and S100A8/A9 production, and the supernatants attracted myeloid cell migration. SERPINB3 promoted MDSC, TAM, and M2 macrophage infiltration contributing to a suppressive environment, which was further augmented locally and systemically upon radiation. Further study revealed a STAT-dependent mechanism, where inhibiting STAT signaling by Ruxolitinib abrogated SERPINB3-mediated CXCL1/8 and S100A8/A9 production. As described herein, a clinical assessment of patients with cervical cancer, a type of squamous cell carcinoma, demonstrated that a combination of high pre-treatment SCCA (SERPINB3) and phosphorylated STAT3 corresponded to a worse outcome with cancer-specific survival at 2 years of about 10% compared to patients with low SCCA and pSTAT3 of 95%.

Briefly, therefore, the present disclosure is directed to compositions and methods for the detection and modulation of a pro-tumor microenvironment to abrogate SERPINB3 radioresistance.

The present teachings include methods for treating squamous cell carcinoma in a patient in need. In one aspect, the methods include administering a therapeutically effective amount of an active agent. In another aspect, the active agent can be configured to modulate STAT-related immunosuppression as an adjuvant to a radiotherapy treatment. In another aspect, the active agent can be a STAT modulator or a SERPINB3 inhibiting agent. In some embodiments, the STAT modulator can be selected from an anti-STAT antibody, a fusion protein, a small molecule, an inhibitory protein, and an sgRNA targeting STAT. In some embodiments, the STAT modulator can be a STAT3 inhibitor. In some embodiments, the SERPINB3 inhibiting agent can be selected from an anti-SERPINB3 antibody, a fusion protein, a small molecule, an inhibitory protein, and an sgRNA targeting SERPINB3. In an exemplary embodiment, the active agent is Ruxolitinib.

The present teachings also include methods for selecting a treatment for a patient with squamous cell carcinoma. In one aspect, the method can include detecting an expression level of SIRPINB3 of the patient. In another aspect, the method includes selecting a treatment comprising radiotherapy and administration of a therapeutically effective amount of an active agent if the expression level of SIRPINB3 falls above a threshold level. In some aspects, detecting the expression level of SIRPINB3 within the tumor comprises obtaining a serum concentration of SCCA from the patient. In some aspects, the threshold level is a serum SCCA concentration of about 9.16 ng/ml. In some aspects, the threshold level is a serum SCCA concentration of about 16.1 ng/ml. In some aspects, the method further includes detecting a pSTAT3 score within a sample of tumor cells stained for pSTAT3 and selecting a treatment comprising radiotherapy and administration of a therapeutically effective amount of the active agent if the expression level of SIRPINB3 falls above a threshold level and the pSTAT3 score falls above a second threshold level. In some aspects, the pSTAT score comprises a percentage of positively-stained tumor cells weighted by an intensity score, wherein the intensity score ranges from about 1 to about 3. In some aspects, the second threshold comprises a pSTAT score of about 100. In one aspect, the active agent can be configured to modulate STAT-related immunosuppression as an adjuvant to a radiotherapy treatment. In another aspect, the active agent can be a STAT modulator or a SERPINB3 inhibiting agent. In some embodiments, the STAT modulator can be selected from an anti-STAT antibody, a fusion protein, a small molecule, an inhibitory protein, and an sgRNA targeting STAT. In some embodiments, the STAT modulator can be a STAT3 inhibitor. In some embodiments, the SERPINB3 inhibiting agent can be selected from an anti-SERPINB3 antibody, a fusion protein, a small molecule, an inhibitory protein, and an sgRNA targeting SERPINB3. In an exemplary embodiment, the active agent is Ruxolitinib.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a schematic of the Structure-function analysis of SERPINB3 interaction with MAPK/STAT to promote chemokine induction & myeloid cell recruitment. Targeted inhibition of the MAPK/STAT pathway reverses SERPINB3-mediated radiation resistance.

FIG. 2A is a graph of SERPINB3 transcript levels in 66 primary patient cervical tumors by RNAseq. Samples are divided into thirds (Low, Intermediate, and High) by FPKM.

FIG. 2B is a heat map of chemokine transcript levels (RPKM scale shown) in primary patient samples by RNAseq.

FIG. 2C is a plot of xCell cell type enrichment analysis of indicated myeloid lineage cells in whole tumor RNAseq comparing Low and High tumors.

FIG. 2D is a pair of graphs of chemokine levels in conditioned media from Caski and SW756 cells at baseline compared to those transfected with vector control (Ctrl) or SERPINB3 (B3).

FIG. 2E is a set of graphs measuring the migration of PBMCs isolated from patients with cervical cancer prior to the start of treatment from a transwell assay using conditioned media from Caski and SW756 cells expressing 83 or Ctrl. Fold change migrated cells with 83-conditioned media compared to Ctrl-conditioned media is shown for overall T and myeloid cells, and subsets of myeloid and T cells.

FIG. 3A is a set of graphs of % myeloid cell subtypes (TAMs=tumor associate macrophages, PMN-MDSCs=polymorphonuclear monocyte-derived suppressor cells, Mo-MDSCs=monocytic MDSCs) in cervical tumor line Caski subcutaneous xenografts with an expression of vector control (Ctrl) or SERPINB3 (B3).

FIG. 3B is a t-distributed stochastic neighbor embedding (t-SNE) plot of myeloid cell (red outline) subtypes (key on the left) summarizing multiparametric flow cytometry data from LL2 subcutaneous tumor xenografts expressing vector control (Ctrl) or mSerpinB3a (B3).

FIG. 3C is a graph of CDS+Tcell:Treg ratio following ex vivo stimulation from LL2 xenograft tumors expressing Ctrl (blue dots), or 83 (red dots) following sham-treatment (Sham) or 1 OGy RT (1 OGy). *=p<0.05, **=p<0.01, =p<0.001.

FIG. 3D is a pair of graphs of CDS or CD4+Ki-67+% of de novo isolated T cells following ex vivo stimulation from LL2 xenograft tumors expressing Ctrl (blue dots), or 83 (red dots) following sham-treatment (Sham) or 1 OGy RT (1 OGy). *=p<0.05, **=p<0.01, ***=p<0.001.

FIG. 3E is a pair of graphs of CDS or CD4+ TNF+/IFNg+ cells following ex vivo stimulation from LL2 xenograft tumors expressing Ctrl (blue dots), or 83 (red dots) following sham-treatment (Sham) or 1 OGy RT (1 OGy). *=p<0.05, **=p<0.01, ***=p<0.001.

FIG. 4A is a pair of graphs of fold-change of CXCL 1/8, S100A8/9 expression in Caski and SW756 cells expressing SERPINB3-wt (B3), SERPINA1 (A1), SERPINB2 (B2), or SERPINB3-A341R.

FIG. 4B is a Western blot of Caski parental cells (WT) or expressing vector control (C) or B3-WT (B3 #1, #2), treated with mock, DMSO, or 1 μM ruxolitinib as indicated.

FIG. 4C set of graphs of CXCL 1/8, S1 OOA8/9 concentration in media supernatant of Caski, vector control (Ctrl) or SERPINB3 (B3) expressing cells treated with mock(−), DMSO, or 1 μM ruxolitinib as indicated. *=p<0.05, **=p<0.01, ***=p<0.001.

FIG. 5 is an immunoblot of indicated antibodies following IP with indicated antibody for SW756 and Caski parental (SW, C), expressing SERPINB3-WT (B3) or CRISPR-Cas9 mediated SERPINB3 KO (B3-KO). The whole cell lysate input is shown in the bottom panel.

FIG. 6 is a schematic of the SCCA1/2 secondary structure with RSL sequence divergence.

FIG. 7A is an experimental design schematic for an in vivo ruxolitinib cancer treatment. C57BL6 mice are randomized to no treatment, vehicle, or ruxolitinib, followed by sham- or 1 OGy×2 RT on days 1 and 2. Tumor, spleen, and peripheral blood will be harvested at day 0 (no treatment), day 7, and 15-20 (based on tumor doubling time), and processed as in FIG. 7B.

FIG. 7B is a schematic of how the samples harvested as in FIG. 7A are processed. The tumor and spleen are homogenized and analyzed for immune cell infiltrate using flow cytometry with indicated profiles, while peripheral blood is processed to serum, and a chemokine array is applied.

FIG. 8A is a graph of SERPINB3 levels of human patients quantified from RNAseq on 66 cervical tumor biopsies collected prior to (chemo)-RT. Patients were divided into three groups based on the distribution of SERPINB3 transcript levels; SERPINB3-low (B3/L, n=22), SERPINB3-intermediate (B3/Int, n=22), and SERPINB3-high (B3/H, n=22) groups. Normalized SERPINB3 transcript in cervical tumor biopsies from RNAseq was distributed by reads per kilobase of transcript per million mapped reads (RPKM).

FIG. 8B is a graph of the xCell Immune Score of the samples described in FIG. 8A. The xCell Immune Score was determined via gene signature-based method using single-sample gene set enrichment analysis with the overall score representing a ranking of tumors in the dataset by lowest (IS of 0) to highest predicted immune infiltrate. B3/H tumors from patients who eventually experienced recurrence (R) compared to those who remained recurrence-free (NR) showed overall higher immune scores then B3/L tumors indicating a potential immune-rich microenvironment. P*<0.05, one-way ANOVA test.

FIG. 8C is a heatmap of cell types from the patients described in FIG. 8A, separated by patients with low (B3/L) and high (B3/H) SERPINB3 expression. Heatmap of enriched immune cell subpopulation was generated through xCell immune infiltrate prediction. Color intensity is proportional to the average xCell score for each population across samples.

FIG. 8D is a graph of CXCL1 expression as a function of SERPINB3 expression, showing a statistically significant correlation between the two factors. Spearman's correlation of SERPINB3 with the expression of CXCL1 was performed using RNAseq from 66 cervical tumor biopsies collected prior to (chemo)-RT.

FIG. 8E is a graph of CXCL8 expression as a function of SERPINB3 expression, showing a statistically significant correlation between the two factors. Spearman's correlation of SERPINB3 with the expression of CXCL8 was performed using RNAseq from 66 cervical tumor biopsies collected prior to (chemo)-RT.

FIG. 8F is a graph of S100A8 expression as a function of SERPINB3 expression, showing a statistically significant correlation between the two factors. Spearman's correlation of SERPINB3 with the expression of S100A8 was performed using RNAseq from 66 cervical tumor biopsies collected prior to (chemo)-RT.

FIG. 8G is a graph of S100A9 expression as a function of SERPINB3 expression, showing a statistically significant correlation between the two factors. Spearman's correlation of SERPINB3 with the expression of S100A9 was performed using RNAseq from 66 cervical tumor biopsies collected prior to (chemo)-RT.

FIG. 8H is a table showing the correlation of SERPINB3 expression with the expression of CXCL1, CXCL8, S100A8, and S100A9 in different human cancer types (BLCA, BRCA, CESC, HNSC, LUSC, PRAD, UCEC). SERPINB3 expression correlated with CXCL1, CXCL8, S100A8, and S100A9 expression in multiple cancer types. Analysis was performed using TCGA PanCancer Atlas and numeric values indicate Spearman's correlation coefficient. BLCA, bladder urothelial carcinoma; BRCA, breast invasive carcinoma; CESC, cervical squamous cell carcinoma and endocervical adenocarcinoma; HNSC, head and neck squamous cell carcinoma; LUSC, lung squamous cell carcinoma; PRAD, prostate adenocarcinoma; UCEC, uterine corpus endometrial carcinoma.

FIG. 9A is a pair of 2 graphs quantifying the fold change of CXCL1, CXCL8, S100A8, and S100A9 in Caski (left) and SW756 (right) human cancer cells when they are genetically modified to express SERPINB3, showing significant increases in all factors in both lines. Cells were transduced with pUltra vector (Caski/Ctrl, SW756/Ctrl) or pUltra-SERPINB3 (Caski/B3, SW756/B3), and CXCL1/8 and S100A8/A9 expression was examined by qPCR.

FIG. 9B is a pair of 2 graphs quantifying the fold change of CXCL1, CXCL8, S100A8, and S100A9 in Caski (left) and SW756 (right) human cancer cells when SERPINB3 is down regulated with shRNA (shB3) or CRISPR-mediated deletion (CRISPR-B3KO), showing significant decreases in all factors in both lines. Caski cells were transfected with scrambled negative control shRNA (Caski/shCtrl) or shRNAs specific SERPINB3 (Caski/shB3); SW756 cells were transduced with CRISPR control vector (SW756/CRISPR-Ctrl) or CRISPR-Cas9 for SERPINB3 knockdown (SW756/CRISPR-B3KO). The expression of CXCL1/8 and S100A8/A9 was examined by qPCR. Gene expression was normalized to GAPDH and fold changes were calculated by comparing to the expression levels in parental cells (Caski WT or SW756 WT).

FIG. 9C is a pair of 2 graphs quantifying the normalized protein concentration of CXCL1, CXCL8, and S100A8/A9 in Caski (left) and SW756 (right) human cancer cells when they are genetically modified to express SERPINB3 (B3), showing significant increases in all factors in both lines. Intracellular chemokine protein was measured by ELISA and the expression levels were normalized to total protein concentration.

FIG. 9D is a pair of 2 graphs quantifying the normalized protein concentration of CXCL1, CXCL8, and S100A8/A9 in Caski (left) and SW756 (right) human cancer cells when they are genetically modified to express SERPINB3 (B3), showing significant increases in all factors in both lines. The supernatant was collected from adherent cells in a monolayer and chemokine secretion was measured by ELISA. Data are presented as mean±SEM of n=3 independent experiments.

FIG. 9E is a graph quantifying the fold-change in T cell and myeloid cell migration in a transwell assay when treated with supernatant from Caski/B3 and SW756/B3 cells, showing an increase of myeloid cell migration but not T cell migration in both lines. PBMC migration towards supernatant collected from cancer cells was examined by Transwell assays and the migrated PMBC populations were analyzed by flow cytometry (FIG. 18A). Fold changes were calculated as the percentage of migrated T and myeloid cells in Caski/B3 or SW756/B3 relative to Caski/Ctrl or SW756/Ctrl supernatant. Data are shown as mean±SEM, ns, no significance.

FIG. 9F is a graph quantifying the fold-change in CD4 T and CD8 T cell migration in a transwell assay when treated with supernatant from Caski/B3 and SW756/B3 cells, showing no increase in migration in either cell type in either cell line. PBMC migration towards supernatant collected from cancer cells was examined by Transwell assays and the migrated PMBC populations were analyzed by flow cytometry (FIG. 18A). Fold changes were calculated as the percentage of migrated T cell subsets in Caski/B3 or SW756/B3 relative to Caski/Ctrl or SW756/Ctrl supernatant. Data are shown as mean±SEM, ns, no significance.

FIG. 9G is a graph quantifying the fold-change in migration in various myeloid cell types (dendritic cells (DC), monocytes, and monocytic and polymorphonuclear myeloid-derived suppressor cells (Mo-MDMCs and PMN-MDSCs)) in a transwell assay when treated with supernatant from Caski/B3 and SW756/B3 cells. PBMC migration towards supernatant collected from cancer cells was examined by Transwell assays and the migrated PMBC populations were analyzed by flow cytometry (FIG. 18A). Fold changes were calculated as the percentage of migrated myeloid cell subsets in Caski/B3 or SW756/B3 relative to Caski/Ctrl or SW756/Ctrl supernatant. Data are shown as mean±SEM, ns, no significance.

FIG. 10A is a graph of the fold change of gene expression of various factors (mCxcl1, mCxcl3, mS100a8, mS100a9, mCxcl9, and mCxcl10) in LL2 murine cancer cells genetically modified to express SerpinB3a (LL2/mb3a) compared to controls expressing an empty vector.

FIG. 10B is a graph of chemokine secretion of various factors (CXCL1, CXCL3, and S100A8/A9) in LL2 murine cancer cells genetically modified to express SerpinB3a (LL2/mb3a) compared to controls expressing an empty vector and unmodified controls.

FIG. 10C is a graph of tumor volume over time of LL2/Ctrl and LL2/mB3a tumors either treated with 10 Gy of radiotherapy (RT) or not (Sham). Tumor growth of C57/BL6 mice with LL2/Ctrl tumors (blue lines) and LL2/B3a tumors (red lines) randomized to receive sham-treated (solid lines) or 10Gy RT at day 14 (dotted lines). Significance was determined by two-way ANOVA.

FIG. 10D is a graph of tumor weight of LL2/Ctrl and LL2/mB3a tumors either treated with 10 Gy of radiotherapy (RT) or not (Sham) 2 days and 7 days post-RT.

FIG. 10E is a visualization of t-distributed stochastic neighbor embedding (viSNE) from an unsupervised analysis of infiltrating immune cells with flow cytometry to cluster CD11b+ myeloid cell subsets based on pre-defined markers. viSNE plots show flow cytometry analysis of total viable CD45+ immune cells from tumors with separate clustering by pre-defined cell surface markers, including Mo-MDSCs (CD11b+Ly6G−Ly6Chigh), PMN-MDSCs (CD11b+Ly6G+), TAM (CD11b+Ly6G−F4/80+), M2 macrophages (CD11b+Ly6G−F4/80+CD163+) and lymphocytes (CD45+CD11b−).

FIG. 10F is a set of images of infiltrating immune cells of LL2/Ctrl and LL2/mB3a cells from tumors pre-RT and 7 days post-RT, which were analyzed as described in FIG. 10E.

FIG. 11A is a graph of normalized protein concentration of CXCL1 in RT-treated (10Gy RT) and untreated (Sham) LL2/Ctrl and LL2/B3a tumors 2 days and 7 days post-RT. Chemokine CXCL1 in tumor homogenates was examined by ELISA. Data were normalized to the protein concentration for each tumor homogenate.

FIG. 11B is a graph of normalized protein concentration of S100A8/A9 in RT-treated (10Gy RT) and untreated (Sham) LL2/Ctrl and LL2/B3a tumors 2 days and 7 days post-RT. Chemokine S100A8/A9 in tumor homogenates was examined by ELISA. Data were normalized to the protein concentration for each tumor homogenate.

FIG. 11C is a graph of the % of Mo-MDSCs in CD45 tumor infiltrating leukocytes (TILs) in RT-treated (10Gy RT) and untreated (Sham) LL2/Ctrl and LL2/B3a tumors 2 days and 7 days post-RT. The graph represents the frequencies of CD11b+Ly6G-Ly6Chigh M-MDSCs in total tumor infiltrating leukocytes (TILs).

FIG. 11D is a graph of the % of PMN-MDSCs in CD45 tumor infiltrating leukocytes (TILs) in RT-treated (10Gy RT) and untreated (Sham) LL2/Ctrl and LL2/B3a tumors 2 days and 7 days post-RT. The graph represents the frequencies of CD11b+Ly6G+ PMN-MDSCs in total tumor infiltrating leukocytes (TILs).

FIG. 11E is a graph of the % of tumor-associated macrophages (TAMs) in CD45 tumor infiltrating leukocytes (TILs) in RT-treated (10Gy RT) and untreated (Sham) LL2/Ctrl and LL2/B3a tumors 2 days and 7 days post-RT. The graph represents the frequencies of CD11b+Ly6G-F4/80+ TAMs in total tumor infiltrating leukocytes (TILs).

FIG. 11F is a graph of the % of M2 macrophages in CD45 tumor infiltrating leukocytes (TILs) in RT-treated (10Gy RT) and untreated (Sham) LL2/Ctrl and LL2/B3a tumors 2 days and 7 days post-RT. The graph represents the frequencies of CD11b+Ly6G-F4/80+CD163+M2 macrophages in total tumor infiltrating leukocytes (TILs).

FIG. 11G is a graph of the % of CD4 T cells in CD45 tumor infiltrating leukocytes (TILs) in RT-treated (10Gy RT) and untreated (Sham) LL2/Ctrl and LL2/B3a tumors 2 days and 7 days post-RT. Cumulative data from FACS analysis of CD3+CD4+ T cells in tumors.

FIG. 11H is a graph of the % of CD8 T cells in CD45 tumor infiltrating leukocytes (TILs) in RT-treated (10Gy RT) and untreated (Sham) LL2/Ctrl and LL2/B3a tumors 2 days and 7 days post-RT. Cumulative data from FACS analysis of CD3+CD4+ T cells in tumors.

FIG. 11I is a graph of the ratio of CD8+ T cells to Treg cells in CD45 tumor infiltrating leukocytes (TILs) in RT-treated (10Gy RT) and untreated (Sham) LL2/Ctrl and LL2/B3a tumors 2 days and 7 days post-RT. The ratio of CD8/Treg represented the infiltrating percentage of CD8+ T cells relative to CD4+CD25+FoxP3+ regulatory T (Treg) cells.

FIG. 12A is a set of flow cytometry plots quantifying the expression of the proliferation marker Ki-67 in CD8+ and CD4+ T cells in tumors grown LL2/Ctrl and LL2/mB3a mice 2 and 7 days after RT (along with an untreated Sham control).

FIG. 12B is a graph quantifying the % of CD8+ T cells in TILs in tumors grown in LL2/Ctrl and LL2/B3a mice 2 and 7 days post-RT (10 Gy) along with untreated controls (Sham). Frequencies of Ki-67+CD8+ T cells in the total infiltrating CD8+ T cell population were analyzed by flow cytometry.

FIG. 12C is a graph quantifying the % of CD4+ T cells in TILs in tumors grown in LL2/Ctrl and LL2/B3a mice 2 and 7 days post-RT (10 Gy) along with untreated controls (Sham). Frequencies of Ki-67+CD4+ T cells in the total infiltrating CD4+ T cell population were analyzed by flow cytometry.

FIG. 12D is a graph quantifying the % of CD8+ T cells taken from LL2/Ctrl and LL2/B3a tumors 2 and 7 days post-RT (10 Gy) that produce IFNγ when stimulated with phorbol 12-myristate 13-acetate (PMA)/ionomycin ex vivo (along with untreated controls: Sham). Intratumoral T cells were stimulated with phorbol 12-myristate 13-acetate (PMA)/ionomycin for 5 h and the expression of IFN-γ and TNF was examined by intracellular staining using flow cytometry. A protein transport inhibitor, brefeldin A, was used to block the protein transport processes and cytokine release. Positive expression was normalized to cells without PMA/ionomycin stimulation (basal levels). Box plot whiskers span minimum and maximum; lines represent the median.

FIG. 12E is a graph quantifying the % of CD4+ T cells taken from LL2/Ctrl and LL2/B3a tumors 2 and 7 days post-RT (10 Gy) that produce IFNγ when stimulated with PMA/ionomycin ex vivo (along with untreated controls: Sham). Cells were stimulated as described in FIG. 12D.

FIG. 12F is a graph quantifying the % of CD8+ T cells taken from LL2/Ctrl and LL2/B3a tumors 2 and 7 days post-RT (10 Gy) that produce TNFα when stimulated with PMA/ionomycin ex vivo (along with untreated controls: Sham). Cells were stimulated as in FIG. 12D.

FIG. 12G is a graph quantifying the % of CD4+ T cells taken from LL2/Ctrl and LL2/B3a tumors 2 and 7 days post-RT (10 Gy) that produce TNFα when stimulated with PMA/ionomycin ex vivo (along with untreated controls: Sham). Cells were stimulated as in FIG. 12D.

FIG. 12H is a graph quantifying the % of CD8+ T cells taken from LL2/Ctrl and LL2/B3a tumors 2 and 7 days post-RT (10 Gy) that produce TNFα and IFNγ when stimulated with PMA/ionomycin ex vivo (along with untreated controls: Sham). Cells were stimulated as in FIG. 12D.

FIG. 12I is a graph quantifying the % of CD4+ T cells taken from LL2/Ctrl and LL2/B3a tumors 2 and 7 days post-RT (10 Gy) that produce TNFα and IFNγ when stimulated with PMA/ionomycin ex vivo (along with untreated controls: Sham). Cells were stimulated as in FIG. 12D.

FIG. 13A is a graph quantifying the % of Mo-MDSCs, PMN-MDSCs, and M2 macrophages in CD45+ cells found in the spleen from tumor-bearing LL2/Ctrl and LL2/B3a mice 2 and 7 days post-RT (10Gy) along with untreated controls (Sham).

FIG. 13B is a graph quantifying spleen weight in tumor-bearing LL2/Ctrl and LL2/B3a mice 2 and 7 days post-RT (10Gy) along with untreated controls (Sham).

FIG. 13C is a graph quantifying CXCL1 plasma concentration in tumor-bearing LL2/Ctrl and LL2/B3a mice 2 and 7 days post-RT (10Gy) along with untreated controls (Sham).

FIG. 13D is a graph quantifying S100A8/A9 plasma concentration in tumor-bearing LL2/Ctrl and LL2/B3a mice 2 and 7 days post-RT (10Gy) along with untreated controls (Sham).

FIG. 13E is a graph quantifying the % of CD8 T cells in CD45+ cells found in the tumor-draining lymph nodes (TDLNs) from tumor-bearing LL2/Ctrl and LL2/B3a mice 2 and 7 days post-RT (10Gy) along with untreated controls (Sham).

FIG. 13F is a graph quantifying the % of CD4 T cells in CD45+ cells found in the tumor-draining lymph nodes (TDLNs) from tumor-bearing LL2/Ctrl and LL2/B3a mice 2 and 7 days post-RT (10Gy) along with untreated controls (Sham).

FIG. 13G is a graph quantifying the % of Treg cells in CD4+ T cells found in the tumor-draining lymph nodes (TDLNs) from tumor-bearing LL2/Ctrl and LL2/B3a mice 2 and 7 days post-RT (10Gy) along with untreated controls (Sham).

FIG. 14A is a graph quantifying the fold change in the signal intensity between Caski/B3 and Caski/Ctrl cells from a human phosphorylation pathway profiling array that contained five cancer-associated pathways—MAPK, AKT, JAK/STAT, NF-κB, and TGF-β.

FIG. 14B is a Western blot for phosphorylated STAT1 (pSTAT1), STAT1, phosphorylated STAT3 (pSTAT3), STAT3, CXCL1, CXCL8, and SCC1 in Caski/Ctrl and Caski/B3 cells treated with DMSO and Ruxolitinib (along with untreated controls).

FIG. 14C is a graph quantifying the fold change in CXCL1 and CXCL8 between Caski/B3, Caski/Ctrl, and Caski/WT cells treated with DMSO or Ruxolitinib (along with untreated controls) as quantified by qPCR.

FIG. 14D is a graph quantifying the fold change in S100A8 and S100A9 between Caski/B3, Caski/Ctrl, and Caski/WT cells treated with DMSO or Ruxolitinib (along with untreated controls) as quantified by qPCR.

FIG. 14E is a graph quantifying the concentration of CXCL1 in Caski/B3, Caski/Ctrl, and Caski/WT cells treated with DMSO or Ruxolitinib (along with untreated controls) as quantified by ELISA.

FIG. 14F is a graph quantifying the concentration of CXCL8 in Caski/B3, Caski/Ctrl, and Caski/WT cells treated with DMSO or Ruxolitinib (along with untreated controls) as quantified by ELISA.

FIG. 14G is a graph quantifying the concentration of S100A8/A9 in Caski/B3, Caski/Ctrl, and Caski/WT cells treated with DMSO or Ruxolitinib (along with untreated controls) as quantified by ELISA.

FIG. 15A is a survival curve of RT-treated cancer patients comparing those with <16.1 ng/mL SCCA serum concentration to those with >=16.1 ng/mL SCCA serum concentration.

FIG. 15B is a survival curve of RT-treated cancer patients comparing those with high pSTAT3 levels to those with low pSTAT3 levels.

FIG. 15C is a survival curve of RT-treated cancer patients comparing those with high pSTAT3/<16.1 ng/mL SCCA serum concentration, low pSTAT3/<16.1 ng/mL SCCA serum concentration, high pSTAT3/>=16.1 ng/mL SCCA serum concentration, and low pSTAT3/>=16.1 ng/mL SCCA serum concentration.

FIG. 16A is a table of the correlation between SERPINB3 expression and immune score in non-recurrent and recurrent patients.

FIG. 16B is a set of tables of multiple t-test analyses for heatmaps of immune cell subsets in B3/High vs B3/Low.

FIG. 16C is a heat map of the expression of CC and CCX chemokines in patients with high (B3/H), intermediate (B3/Int), and low (B3/L) SERPINB3 expression as quantified by RNA-seq.

FIG. 16D is a set of graphs of the correlation between SERPINB3 and CXCL1, CXCL8, S100A8, or S100A9 expression in the TCGA-CESE (cervical squamous cell carcinoma and endocervical adenocarcinoma, n=306) patient dataset.

FIG. 16E is a graph quantifying SERPINB3 mRNA levels in patients across cancer types, showing elevated levels for bladder, cervical, esophageal, head and neck, and lung cancers.

FIG. 17A is a Western blot showing a successful genetic modification of SERPINB3 expression in human cervical cancer cells (Caski and SW756).

FIG. 17B is a set of 3 graphs comparing the expression of CXCL1, CXCL8, S100A8, and S100A9 in HPV-positive C33A, C33A/Ctrl, and C33A/B3 cells.

FIG. 18A is a schematic showing the experimental design of the chemotactic response of human peripheral blood mononuclear cells (PBMCs), obtained from seven cervical cancer patients prior to treatment, to the chemokines secreted by cancer cells with high SERPINB3 expression using a transwell assay and flow cytometry to characterize migrated cells.

FIG. 18B is a graph quantifying the % of migrated CD3+ T cells, CD11b+ myeloid cells, CD4 T cells, CD8 T cells, DCs, monocytes, Mo-MDSCs, and PMN-MDSCs treated with supernatant collected from Caski/Ctrl, Caski/B3, SW756/Ctrl, and SW756/B3 cells as quantified by the assay described in FIG. 18A.

FIG. 18C is a table that quantifies the fold change in migration between cells treated with SERPINB3-modified cell supernatant (Caski/B3, SW756/B3) and cells treated with control cell supernatant (Caski/Ctrl, SW756/Ctrl).

FIG. 19A is a schematic illustrating how flow cytometry was used to characterize tumor-infiltrating immune cells from Caski/Ctrl and Caski/B3 tumors.

FIG. 19B is a pair of graphs quantifying tumor volume and tumor weight over time of Caski/Ctrl and Caski/B3 tumors.

FIG. 19C is a graph quantifying the % of CD11b+ cells (infiltrating CD11b+ myeloid cells) in Caski/Ctrl and Caski/B3 tumors 22 and 40 days after tumor inoculation.

FIG. 19D is a set of graphs quantifying the % of DCs, TAMs, Mo-MDSCs, M2-like TAMs, PMN-MDSCs, and B cells in CD45 TILs from Caski/Ctrl and Caski/B3 tumors 22 and 40 days after tumor inoculation.

FIG. 20A is a table of murine chemokine expression levels in TC1 and LL2 cells.

FIG. 20B is an immunoblot and associated graph of SERPINB3a expression levels in LL2 WT, LL2/Ctrl, and LL2/mB3a cells.

FIG. 20C is a pair of graphs of chemokine expression in LL2 cells transduced with a pLV-C-GFPSpark vector with mSerpinb3a expression (LL2/B3a) or a control pLV-C-GFPSpark vector (LL2/Ctrl) were examined by qPCR (first sheet) and ELISA (second sheet). Gene expression was normalized to mGapdh and fold changes were calculated by comparing to the expression levels in LL2 parental cells. Data are shown as mean±SEM of n=4 independent experiments, *P<0.05, **P<0.01, ***P<0.001 using the Mann-Whitney test.

FIG. 21A is a graph of tumor weights at 2-day and 7-day post-RT.

FIG. 21B is a schematic illustrating how flow cytometry was used to analyze infiltrating immune cells from RT-treated LL2/Ctrl and LL2/mB3a tumors (as well as untreated controls).

FIG. 21C is a graph quantifying the ratio of M2 macrophages to TAMs in RT-treated LL2/Ctrl and LL2/mB3a tumors (as well as untreated controls).

FIG. 21D is a graph quantifying the % DCs in CD45 TILs in RT-treated LL2/Ctrl and LL2/mB3a tumors (as well as untreated controls).

FIG. 22 is a schematic illustrating how flow cytometry was used to analyze immune cells from the spleen and TDLNs from RT-treated LL2/Ctrl and LL2/mB3a tumors (as well as untreated controls).

FIG. 23 is a set of graphs of fold-change in expression between LL2/mB3a-tumor expression of JAK/STAT/TGFβ, AKT, and MAPK/NFkB pathways as determined by a phosphorylation protein assay.

FIG. 24 is a graph of doubling time (DT) from days 7-14 and days 14-21, calculated by dividing the natural logarithm of 2 by the exponent of growth. DT=duration×ln2/ln(v2/v1). Data are shown as mean±SEM and each dot represents a biologically independent animal; ns, not significant; *P<0.05 using one-way ANOVA with Tukey's post hoc test.

FIG. 25A is an immunoblot (first sheet) and quantification (second sheet) that shows the inhibition of STAT1/3 phosphorylation after treating SW756 parental cells (WT), SW756/Ctrl (C), and SW756/B3 (B3) with 1 uM Ruxolitinib for 48 h.

FIG. 25B is a set of immunoblots of Caski cells that were transfected with scramble shRNA (shC) or SERPINB3 shRNA (shB3). Immunoblotting shows the reduced STAT1/3 phosphorylation by the knockdown of SERPINB3.

FIG. 25C is a set of graphs of CXCL1/8 and S100A8/A9 expression in Caski/SW756 cells. Cells were treated with 1 uM Ruxolitinib and the expression CXCL1/8 and S100A8/A9 mRNA was examined by qPCR. Gene expression was normalized to GAPDH and fold changes were calculated by comparing to the expression levels in parental cells (WT). Data are shown as mean±SEM of n=3, *P<0.05, **P<0.01, ***P<0.001 using one-way ANOVA with Tukey's post hoc test.

FIG. 25D is an immunoblot that shows the knockdown of STAT1/3 by siRNA in SW756 cells.

FIG. 25E is a set of graphs of the expression CXCL1/8 and S100A8/A9 mRNA by qPCR after STAT1/3 knockdown. Gene expression was normalized to GAPDH and fold changes were calculated by comparing to the expression levels in SW756/Ctrl transfected with negative control siRNA. Data are shown as mean±SEM of n=3, *P<0.05, **P<0.01, **P<0.001 using one-way ANOVA with Tukey's post hoc test.

FIG. 26 is a table of the patient characteristics used in the present disclosure.

FIG. 27 is a table of the antibodies used in the present disclosure.

FIG. 28 is a table of the primers used in the present disclosure.

FIG. 29A is a set of flow cytometry histograms characterizing splenic T+intratumoral myeloid cells after stimulation. Myeloid cell subtypes were isolated from tumors and cocultured with CellTrace-labeled splenic T cells at a ratio of 1:1 for 4 days. CD3/28 antibodies were added to stimulate T cell proliferation. Histograms show the percentage of divided cells. Percentages of suppression were calculated by comparing to the dilution of CellTrace in splenic T cells without myeloid cell co-culture. Data are shown as mean±SEM; *P

FIG. 29B is a graph derived from the histograms in FIG. 29A quantifying % suppression of myeloid subtypes. Myeloid cell subtypes were isolated from tumors and co-cultured with CellTrace-labeled splenic T cells at a ratio of 1:1 for 4 days. CD3/28 antibodies were added to stimulate T cell proliferation. Percentages of suppression were calculated by comparing to the dilution of CellTrace in splenic T cells without myeloid cell co-culture. Data are shown as mean±SEM; *P<0.05 using the Mann-Whitney test.

FIG. 30A is a set of flow cytometry histograms and associated graphs quantifying the % proliferating cells from LL2/Ctrl and LL2/mB3a tumors that were either sham-treated or treated with RT. CellTrace-labeled intratumoral T cells were stimulated with anti-CD3/28 antibody for 4 days and cell proliferation was determined by the dilution of CellTrace.

FIG. 30B is a pair of graphs quantifying PD-1 and CTLA-4 expression in T cells from LL2/Ctrl and LL2/mB3a tumors either sham-treated or treated with RT. The expression of PD-1 and CTLA-4 was examined by flow cytometry, shown in mean fluorescence intensity (MFI). Data are shown as mean±SEM and each dot represents a biologically independent sample; * indicates comparisons between LL2/Ctrl and LL2/m; t indicates comparisons between sham-treated and RT; *P<0.005, **P<0.01. ***P<0.001 using one-way ANOVA with Tukey's post hoc test.

FIG. 31A is a set of representative flow cytometry plots that show the depletion of CD11b+ cells in tumors and spleens on day 15 and day 21 post-tumor inoculation, gated on CD45+CD11b+ cells.

FIG. 31B is a graph showing changes in the tumor volume of LL2/Ctrl tumors (blue line), and LL2/B3a tumors treated with CD11b antibody (red dotted line) or IgG2b antibody (red solid line). Significance was determined by two-way ANOVA, ***P<0.001.

FIG. 31C is a graph of the numbers of infiltrating CD8+ T cells in 5×10⁵ total tumor cells that were determined by flow cytometry. Data are shown as mean±SEM and each dot represents a biologically independent sample; *P<0.05, **P<0.01, ***P<0.001 using one-way ANOVA with Tukey's post hoc test.

FIG. 31D is a representative flow cytometry plot and associated graph quantifying % proliferating CD8 T cells from tumors described in FIG. 31B. CellTrace-labeled intratumoral T cells were stimulated with anti-CD3/28 antibody for 4 days and cell proliferation was determined by the dilution of CellTrace. Data are shown as mean±SEM and each dot represents a biologically independent sample; *P<0.05, **P<0.01 ***P<0.001 using one-way ANOVA with Tukey's post hoc test.

FIG. 31E is a pair of representative histograms of intracellular cytokine staining of granzyme B and perforin in CD8+ T cells, along with associated graphs quantifying the % of CD8 T cells positive for granzyme B and perforin. Data are shown as mean±SEM and each dot represents a biologically independent sample; *P<0.05, **P<0.01, ***P<0.001 using one-way ANOVA with Tukey's post hoc test.

FIG. 31F is a pair of graphs quantifying the expression of PD-1 and CTLA-4 by flow cytometry, shown in mean fluorescence intensity (MFI). Data are shown as mean±SEM and each dot represents a biologically independent sample; *P<0.05, **P<0.01, ***P<0.001 using one-way ANOVA with Tukey's post hoc test.

FIG. 32A is a graph showing the knockdown of Serpinb3a in tumors by qPCR. Gene expression was normalized to mGapdh and shown as a log 2 fold change.

FIG. 32B is a plot of tumor growth curves, as well as associated photos, of LL2/B3 treated with negative control siRNA (siNC, red lines) and Serpinb3a siRNA (siB3, green lines) with or without radiation treatment (sham-solid lines; RT-dotted lines). Significance was determined by two-way ANOVA, ***P<0.001.

FIG. 32C is a pair of graphs quantifying the normalized concentration of chemokines, CXCL1 and S100A8/A9 in tumor homogenates from FIG. 2B by ELISA. Data were normalized to the protein concentration for each tumor homogenate. Data are shown as mean±SEM and each dot represents a biologically independent sample; *P<0.05, **P<0.01, ***P<0.001 using one-way ANOVA with Tukey's post hoc test.

FIG. 32D is a set of graphs of cumulative data from FACS analysis that show the frequencies of immune cell populations including CD11b+Ly6G-Ly6Chigh Mo-MDSCs, CD11b+Ly6G+ PMN-MDSCs, CD11b+Ly6G−F4/80+ TAMs, and CD11b+Ly6G−F4/80+CD163+M2 macrophages in total tumor infiltrating leukocytes (TILs) from the tumors in FIG. 32B. Data are shown as mean±SEM and each dot represents a biologically independent sample; *P<0.05, **P<0.01, ***P<0.001 using one-way ANOVA with Tukey's post hoc test.

FIG. 32E is a pair of graphs of cumulative data from FACS analysis that show the frequencies of immune cell populations CD3+CD8+ T cells in total tumor infiltrating leukocytes (TILs) and the ratio of CD3+CD8+ T cells to CD4+CD25+Foxp3+ Treg cells. Data are shown as mean±SEM and each dot represents a biologically independent sample; *P<0.05, **P<0.01, ***P<0.001 using one-way ANOVA with Tukey's post hoc test.

FIG. 32F is a pair of graphs quantifying the percent of CD8 T cells from the tumors in FIG. 32B that are positive for granzyme B and perforin. Intracellular cytokine staining of granzyme B and perforin in CD8+ T cells was analyzed by flow cytometry. Data are shown as mean±SEM and each dot represents a biologically independent sample; *P<0.05, **P<0.01, ***P<0.001 using one-way ANOVA with Tukey's post hoc test.

FIG. 32G is a representative flow cytometry trace and associated graph quantifying % proliferating CD8 T cells in tumors in FIG. 32B. CellTrace-labeled intratumoral T cells were stimulated with anti-CD3/28 antibody for 4 days and cell proliferation was determined by the dilution of CellTrace. Data are shown as mean±SEM and each dot represents a biologically independent sample; *P<0.05, **P<0.01, ***P<0.001 using one-way ANOVA with Tukey's post hoc test.

FIG. 32H is a pair of graphs quantifying the expression of PD-1 and CTLA-4 in CD8 T cells from the tumors in FIG. 32B by flow cytometry, shown in mean fluorescence intensity (MFI). Data are shown as mean±SEM and each dot represents a biologically independent sample; *P<0.05, **P<0.01, ***P<0.001 using one-way ANOVA with Tukey's post hoc test.

FIG. 33A is a graph quantifying the fold-change activation of JAK/STAT pathway-associated proteins using a phosphorylation antibody array. Fold changes in phosphorylation were calculated by normalizing the intensity to the expression levels in Caski parental cells and comparing the phosphorylation intensity in Caski/B3 to the levels in Caski/Ctrl cells. The red line indicates fold change 2 and the blue line indicates fold change s 0.5.

FIG. 33B is an immunoblot (first sheet) and quantification (second sheet) of the immunoblot that shows the inhibition of STAT1/3 phosphorylation after treating Caski parental cells (WT), Caski/Ctrl (C), and Caski/B3 (B3 #1, B3 #2) with 1 uM Ruxolitinib for 48 h. Data are shown as mean±SEM of n=3, *P<0.05, **P<0.01 ***P<0.001 using one-way ANOVA with Tukey's post hoc test.

FIG. 33C is a set of graphs of concentration of CXCL1, CXCL8, and S100A8/A9 secreted by Caski/WT, Caski/Ctrl, and Caski/B3 treated with 1 uM Ruxolitinib, and the secretion CXCL1, CXCL8, and S100A8/A9 was examined by ELISA. Data are shown as mean±SEM of n=3, *P<0.05, **P<0.01, ***P<0.001 using one-way ANOVA with Tukey's post hoc test.

FIG. 33D is an immunoblot showing the knockdown of STAT1/3 by siRNA in Caski cells.

FIG. 33E is a set of graphs quantifying the expression of CXCL1/8 and S100A8/A9 mRNA by qPCR after STAT1/3 knockdown. Gene expression was normalized to GAPDH and fold changes were calculated by comparing to the expression levels in Caski/Ctrl transfected with negative control siRNA. Data are shown as mean±SEM of n=3, *P<0.05, **P<0.01, ***P<0.001 using one-way ANOVA with Tukey's post hoc test.

FIG. 33F is an immunoblot showing the expression of pSTAT1/3 in the nucleus (Nuc), cytoplasm (Cyt), and total cell lysates.

FIG. 33G is an immunoprecipitation using anti-JAK1 antibody that shows increased interaction with STAT1 and STAT3 in Caski/B3 and SW756/B3 compared with Caski/Ctrl and SW756/Ctrl cells.

FIG. 34A is a set of representative immunohistochemistry images of mouse tumors treated with negative control or Serpinb3a siRNA stained with pSTAT3. Box plots show pSTAT3 staining score from 8-12 representative fields each for n=6 or 7 mice per group. Box plot whiskers span minimum and maximum; lines represent the median.

FIG. 34B is a set of representative immunohistochemistry images of mouse tumors treated with negative control or Serpinb3a siRNA stained with CD11b. Box plots show regions of CD11b positive staining from 8-12 representative fields each for n=6 or 7 mice per group. Box plot whiskers span minimum and maximum; lines represent the median.

FIG. 34C is a set of representative images of pSTAT3 and CD11b staining from patients with SCCA< or ≥9.16 ng/ml.

FIG. 34D is a graph of phosphorylated STAT3 staining score (histoscore) in patients with serum SCCA<9.16 ng/ml vs. SCCA≥9.16 ng/ml.

FIG. 34E is a graph of myeloid cell marker CD11b staining in patients with serum SCCA< or ≥9.16 ng/ml with pSTAT3 histoscore<100 (low) or ≥100 (high). Each dot represents an individual patient. Data are shown as mean±SEM, using the Mann-Whitney test.

### DETAILED DESCRIPTION OF THE INVENTION

The present disclosure is based, at least in part, on the discovery that overexpression of SERPINB3 causes radioresistance in squamous cell carcinoma by STAT-related immunosuppression. In various aspects, compositions and methods for the detection of SERPINB3 and other immune system-modulating pathways using nucleic acid-based assays are described. In various other aspects, compositions and methods for the modulation of the tumor microenvironment with immunomodulators, including but not limited to STAT modulators are disclosed.

It has previously been demonstrated that patients with persistently high levels of SCCA before treatment and throughout the course of definitive RT had an increased risk of recurrence and death. Prospective cohort studies also showed the prognostic value of SCCA for monitoring the response to RT and clinical outcome post-RT in cervical cancer patients. Given the unfavorable outcomes of patients with high SERPINB3 expression, it was hypothesized that SERPINB3 promotes immune evasion by modulating suppressive immune responses that alter the tumor microenvironment and impede RT-induced anti-tumor immunity. By characterizing cancer cells with high SERPINB3 expression within the context of the tumor microenvironment, the methods described herein provide a better understanding of treatment responsiveness and a strategy to improve tumor control. By analysis of RNA-sequencing (RNAseq) data to predict immune infiltrate, enriched myeloid cell gene signature and immunosuppressive chemokine gene expression were found in tumors with high SERPINB3 expression. In vitro and in vivo assays suggested that chemokines induced by SERPINB3 contributed to myeloid cell migration and led to a resistant environment to RT, in which high MDSCs, tumor-associated macrophages (TAMs), regulatory T cell (Treg) infiltration, and impaired T cell functionality were observed. It was further discovered that SERPINB3 functioned through STAT signaling and therefore, inhibiting STAT activity significantly reduced SERPINB3-associated suppressive chemokine production. Here, the clinical importance and regulatory function of SERPIN3 in the tumor microenvironment that facilitated tumor progression are presented.

One aspect of the present disclosure provides methods for the detection of SERPINB3 expression that causes radioresistance in cancer using biological assays, which include but are not limited to RNA sequencing.

Another aspect of the present disclosure includes compositions and methods of use for STAT and SERPINB3 modulators.

Another aspect of the present disclosure includes the use of STAT and/or SERPINB3 modulators to improve radiotherapy by abrogating SERPINB3-modulated immunosuppression in cancer.

SERPINB3 and STAT Modulation Agents

As described herein, SERPIN3 expression has been implicated in various diseases, disorders, and conditions, including radioresistance in cancer patients. As such, modulation of SERPINB3 (e.g., through modulation of STAT) can be used for the treatment of such conditions. A STAT modulation agent can modulate tumor response or induce or inhibit the immune system and/or a pro-tumor microenvironment. STAT modulation can comprise modulating the expression of SERPINB3 on cells, modulating the quantity of cells that express SERPINB3, or modulating the quality of the SERPINB3-overexpressing cells.

In various aspects, STAT modulation agents can be any composition or method that can modulate STAT expression in cells without limitation. For example, a STAT modulation agent can be an activator, an inhibitor, an agonist, or an antagonist. As another example, STAT modulation can be the result of gene editing.

In some aspects, the STAT modulation agent can be an antibody.

In various aspects, STAT modulating agents can be an agent that induces or inhibits progenitor cell differentiation into STAT (and therefore SERPINB3) expressing cells. For example, the STAT modulation agent used to inhibit STAT may be Ruxolitinib.

In various additional aspects, a SERPINB3 modulation agent can be any of the above embodiments of the STAT modulation agents described above whose mechanism of action is STAT-independent.

SIRPINB3 STAT Signal Reduction, Elimination, or Inhibition by Small Molecule Inhibitors, shRNA, siRNA, or ASOs

As described herein, a STAT modulation agent can be used as at least a portion of a cancer therapy. In various aspects, the STAT modulation agent can be used to reduce/eliminate or enhance/increase SERPINB3 signals. For example, a STAT modulation agent can be a small molecule inhibitor of STAT. As another example, a STAT modulation agent can be a short hairpin RNA (shRNA). As another example, a STAT modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

SERPINB3 and STAT Inhibiting Agent

One aspect of the present disclosure provides for targeting of STAT and/or SERPINB3, its receptor, or its downstream signaling. The present disclosure provides methods of treating or preventing radioresistance associated with a cancer treatment based on the discovery that SERPINB3 μlays a central role in a pro-tumor microenvironment through overexpression of SERPINB3.

As described herein, inhibitors of SERPINB3 (e.g., antibodies, fusion proteins, small molecules) can reduce or prevent radioresistance in cancer. A SERPINB3 inhibiting agent can be any agent that can inhibit SERPINB3, downregulate SERPINB3, or knockdown SERPINB3.

As an example, a STAT-inhibiting agent can inhibit SERPINB3 signaling.

As another example, the SERPINB3 inhibiting agent can be an anti-SERPINB3 antibody, wherein the anti-SERPINB3 antibody prevents binding of SERPINB3 to its receptor or prevents activation of SERPINB3 and downstream signaling.

As another example, the SERPINB3 inhibiting agent can be a fusion protein. For example, the fusion protein can be a decoy receptor for SERPINB3. Furthermore, the fusion protein can comprise a mouse or human Fc antibody domain fused to the ectodomain of SERPINB3.

As another example, a SERPINB3 inhibiting agent can be Ruxolitinib, which has been shown to be a potent and specific inhibitor of STAT signaling.

As another example, a SERPINB3 inhibiting agent can be an inhibitory protein that antagonizes SERPINB3 or STAT. For example, the SERPINB3 inhibiting agent can be a viral protein, which has been shown to antagonize SERPINB3 or STAT.

Other non-limiting examples of suitable STAT inhibiting agents are STAT3 inhibitors, including S31-201, WP1066, Resveratrol (SRT501), Stattic, Niclosamide (BAY2353), Morusin, STAT3-IN-1, inS3-54-A18, C188-9, BP-1-102, SH-4-54, and Cryptotanshinone.

As another example, a SERPINB3 inhibiting agent can be a short hairpin RNA (shRNA) or a short interfering RNA (siRNA) targeting SERPINB3 or STAT.

As another example, a SERPINB3 inhibiting agent can be an sgRNA targeting SERPINB3 or STAT.

Methods for preparing a SERPINB3 or STAT inhibiting agent (e.g., an agent capable of inhibiting SERPINB3 and/or STAT signaling) can comprise the construction of a protein/Ab scaffold containing the natural SERPINB3 receptor as a SERPINB3 neutralizing agent; developing inhibitors of the SERPINB3 receptor “down-stream”; or developing inhibitors of the SERPINB3 production “up-stream”.

Inhibiting SIRPINB3 and/or STAT can be performed by genetically modifying SIRPINB3 and/or STAT in a subject or genetically modifying a subject to reduce or prevent expressions of their genes, such as through the use of CRISPR-Cas9 or analogous technologies, wherein, such modification reduces or prevents SIRPINB3 and/or STAT expression.

Chemical Agents:

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀ alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated.

The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein.

Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterions. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions.

Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| | Ile, Val, Met, Ala, |
| Leu (L) | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| | Ile, Leu, Met, Phe, |
| Val (V) | Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editinq

As described herein, SIRPINB3 and/or STAT signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of SIRPINB3 and/or STAT by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double-strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for the modulating agents to target cells by the removal of SIRPINB3 and/or STAT signals.

For example, the methods as described herein can comprise a method for altering a target polynucleotide sequence in a cell comprising contacting the polynucleotide sequence with a clustered regularly interspaced short palindromic repeats-associated (Cas) protein.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Therapeutic Methods

Also provided is a process of treating, preventing, or reversing cancer in a subject in need of administration of a therapeutically effective amount of a tumor microenvironment modulating agent, so as to, for example, improve response to radiotherapy in cancer subjects.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a tumor microenvironment modulating agent (which can include immunomodulatory agents such as STAT inhibitors) is, for example, an amount that would cause the desired therapeutic effect in a subject while minimizing undesired side effects. In various embodiments, an effective amount of the tumor microenvironment modulating agent described herein can substantially inhibit tumor-based immunosuppression, slow the progress of progression, or limit the development of radioresistance.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a tumor microenvironment modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to improve response to radiotherapy in cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the tumor microenvironment modulating agent (for example, a SIRPINB3 or STAT modulating agent) can occur as a single event or over a time course of treatment. For example, the modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancer, and potentially other diseases associated with immunosuppression.

A tumor microenvironment modulating agent (including immunomodulatory agents like STAT modulators) can be administered simultaneously or sequentially with another agent, such as an antibiotic, an anti-inflammatory, a chemotherapy, or another agent. For example, a tumor microenvironment modulating agent can be administered simultaneously with another agent, such as an antibiotic or an anti-inflammatory. Simultaneous administration can occur through the administration of separate compositions, each containing one or more of a tumor microenvironment modulating agent, an antibiotic, an anti-inflammatory, or another agent. Simultaneous administration can occur through the administration of one composition containing two or more of an immunomodulatory agent, an antibiotic, an anti-inflammatory, or another agent. The immunomodulatory agent (which is also a tumor microenvironment modulator can be administered sequentially with an antibiotic, an anti-inflammatory, or another agent. For example, an immunomodulatory agent can be administered before or after the administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

Screening

Also provided are methods for screening.

The subject methods find use in the screening of a variety of different candidate molecules (e.g., potentially therapeutic candidate molecules).

Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a mixture of immunomodulatory factors, chemotherapy, and tumor targeting moieties. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrates, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

### Example 1—SerpinB3 Promotes STAT-Pathway Activation, Enhanced Chemokine Production, Recruitment of Myeloid Cells, and Radioresistance

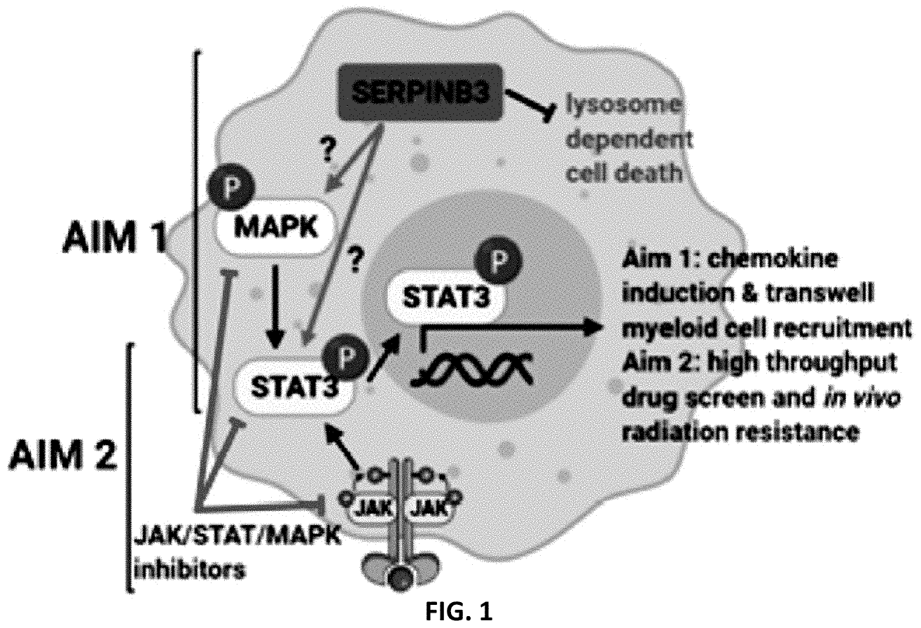

SERPINB3-high tumors are enriched for TAMs and MDSCs. SERPINB3 expression results in increased activation of STAT transcription factors, and expression and secretion of myeloid cell chemoattractants (FIG. 1). Inhibition of JAK/STAT with the FDA-approved anti-JAK drug ruxolitinib abolished SERPINB3-dependent chemokine expression, while a reactive-site-loop mutant of SERPINB3 (A341R) within the RSL domain, eliminating protease inhibitory function did not. Proximity labeling using the biotinylation enzyme TurboID fused to SERPIN83 identified members of the STAT and MAPK pathways. Coimmunoprecipitation confirmed the interaction between SERPIN83 and STAT3, and SERPIN83 and ERK1/2.

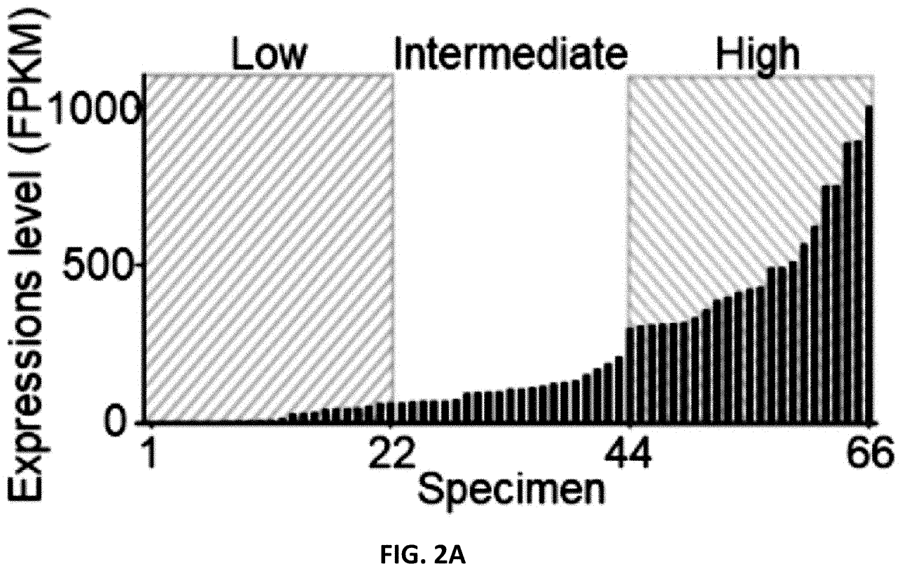

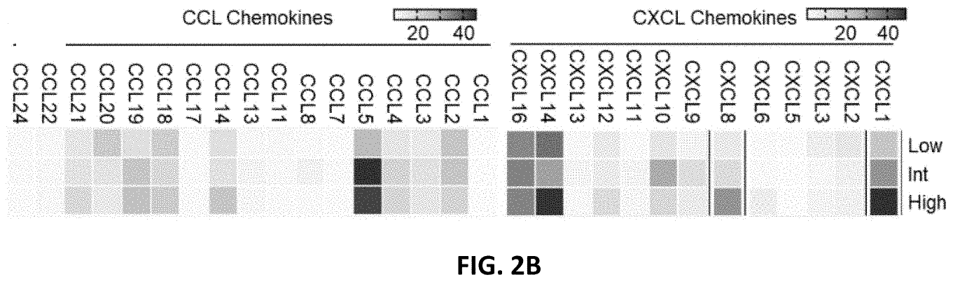

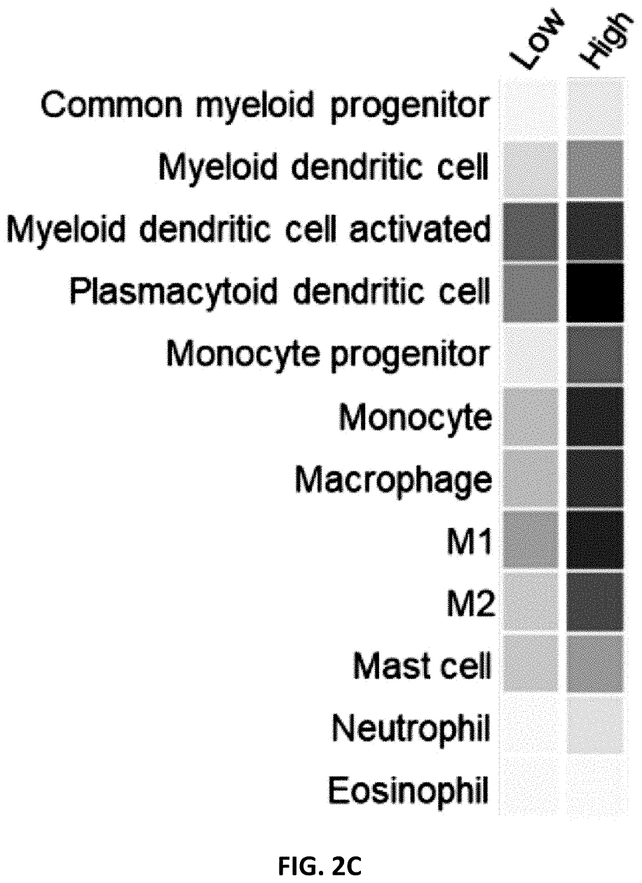

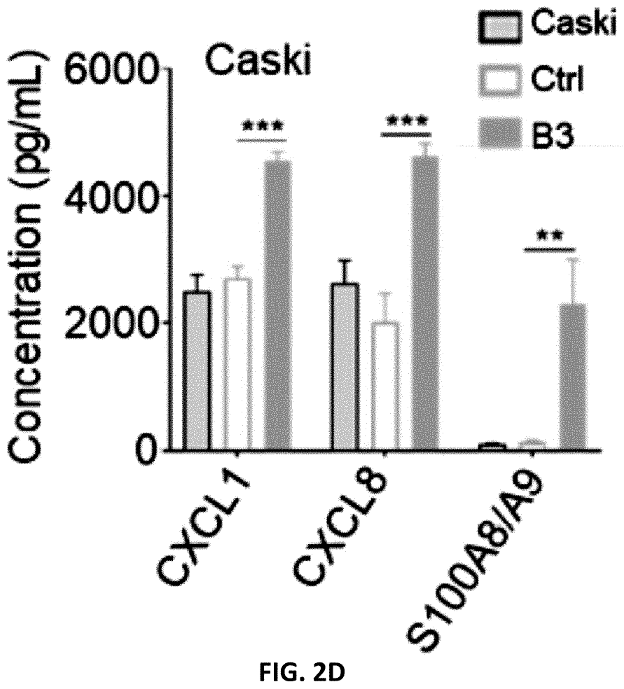

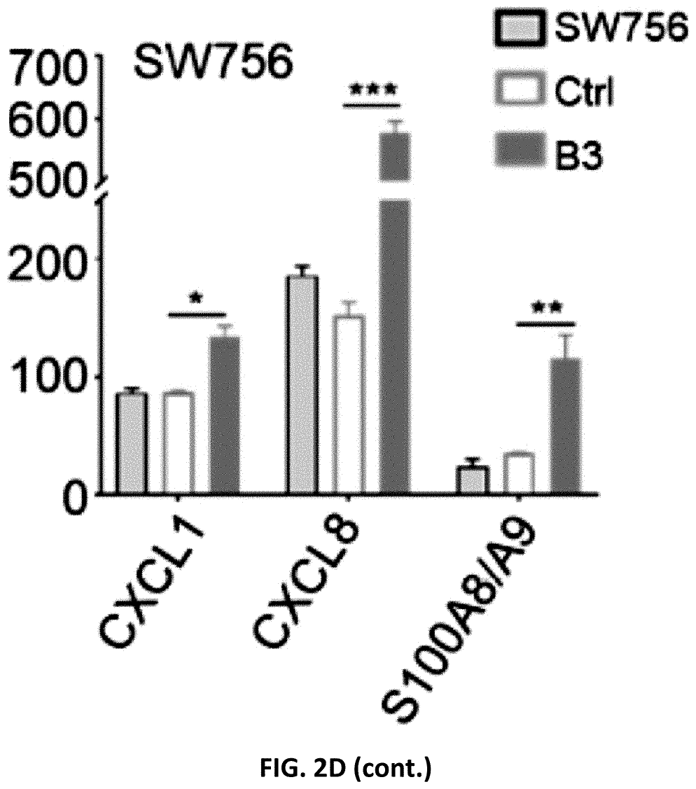

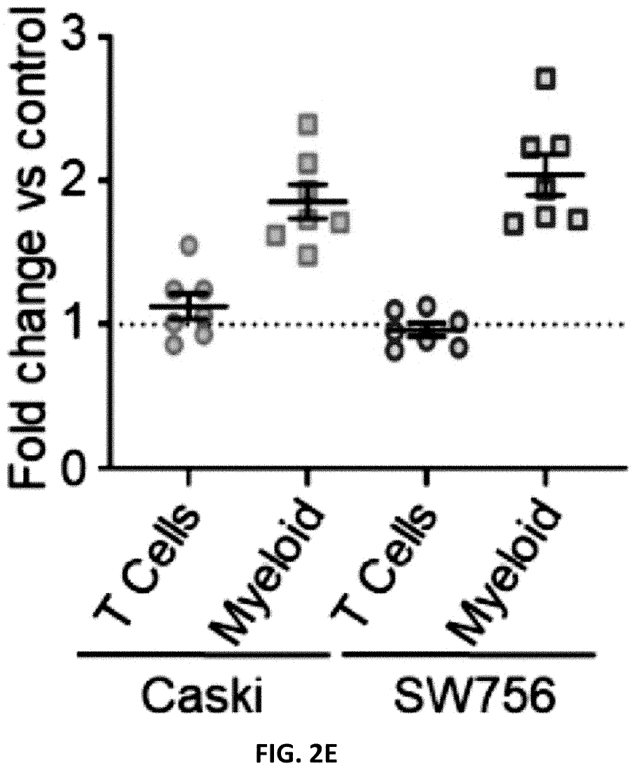

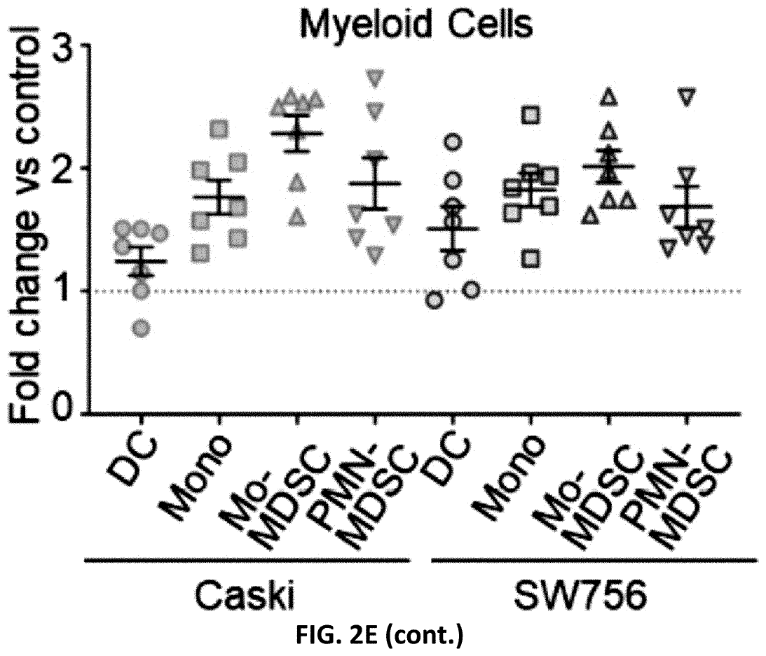

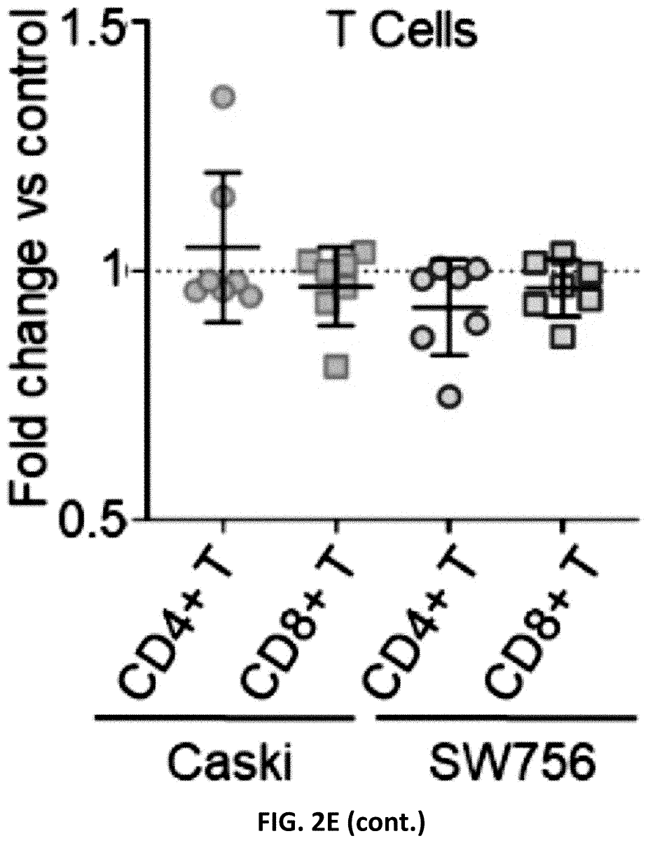

RNAseq analysis of primary cervical tumor specimens revealed an association between high SERPINB3 expression and increased expression of myeloid cell chemoattractants CXLC1, CXCL8, S1 OOA8, and S100A9, as well as higher predicted representation of myeloid cells (FIG. 2A-C). In two cell culture models, cervix tumor cells engineered to express SERPINB3 secreted much higher levels of these chemokines, and conditioned media from these cells induced migration of myeloid cells through a Transwell insert when cultured with peripheral blood mononuclear cells (PBMCs) obtained from patients with cervical cancer (FIG. 2D-E).

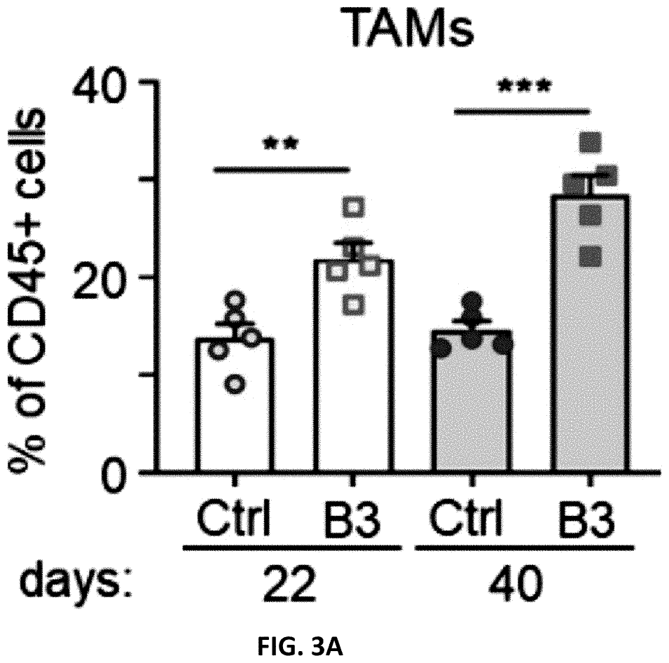

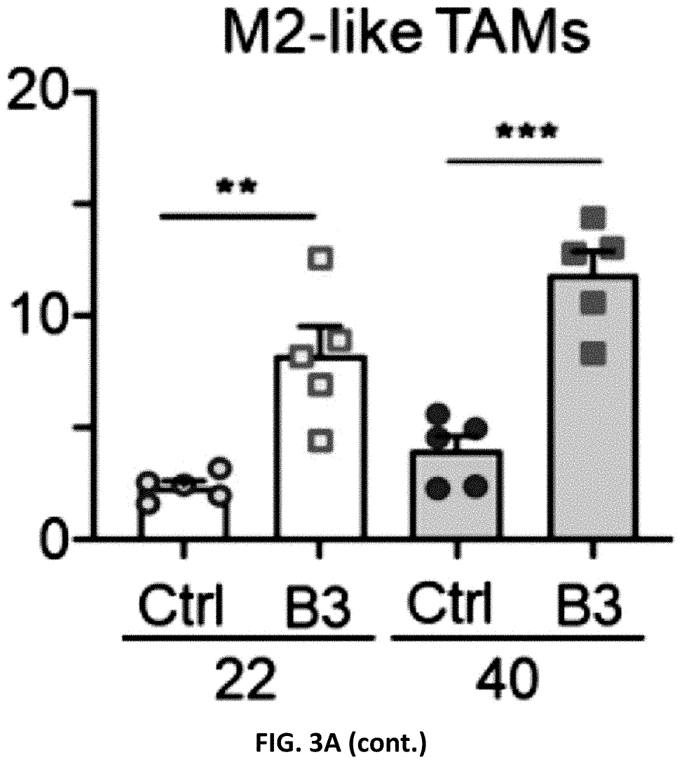

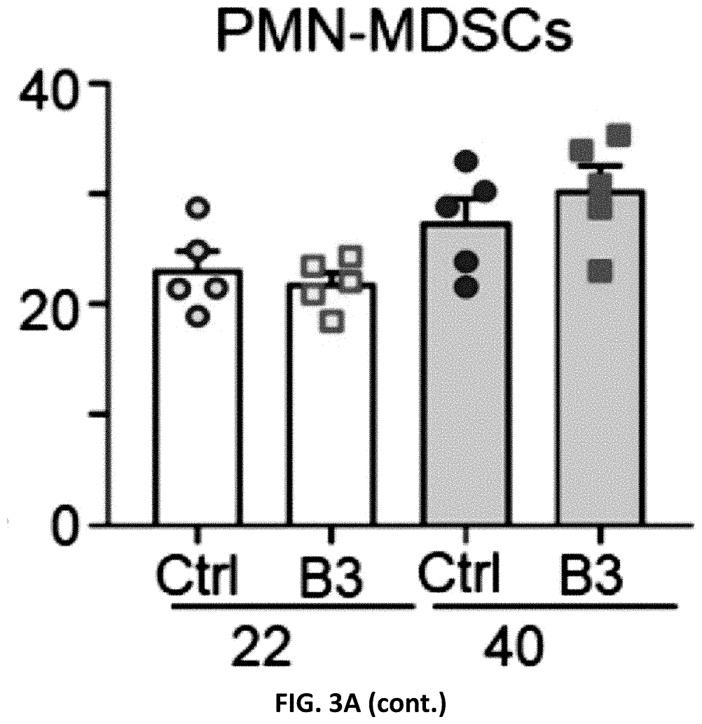

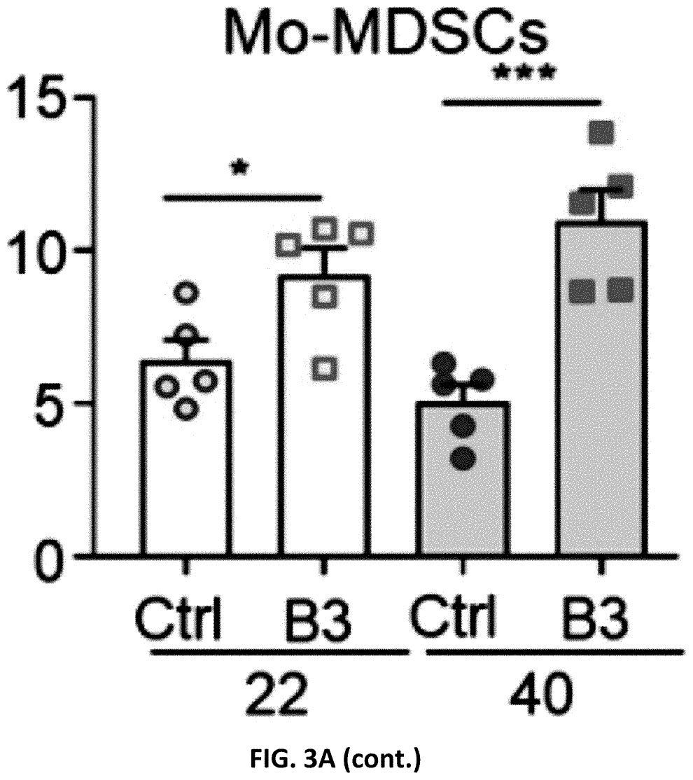

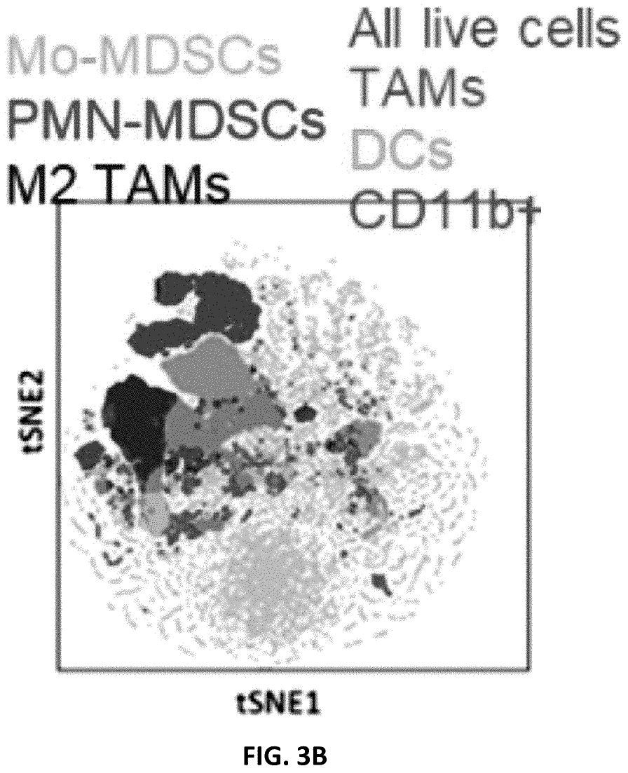

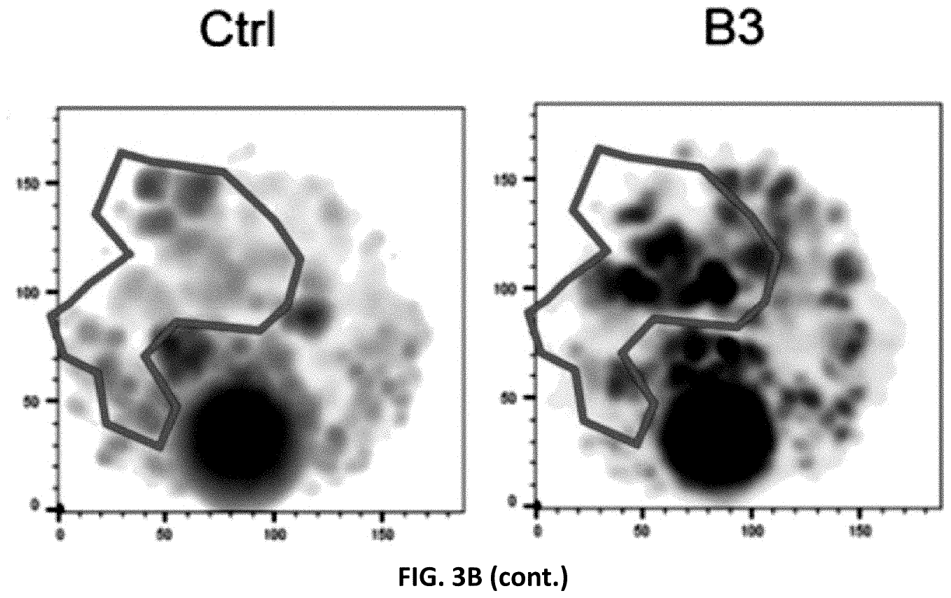

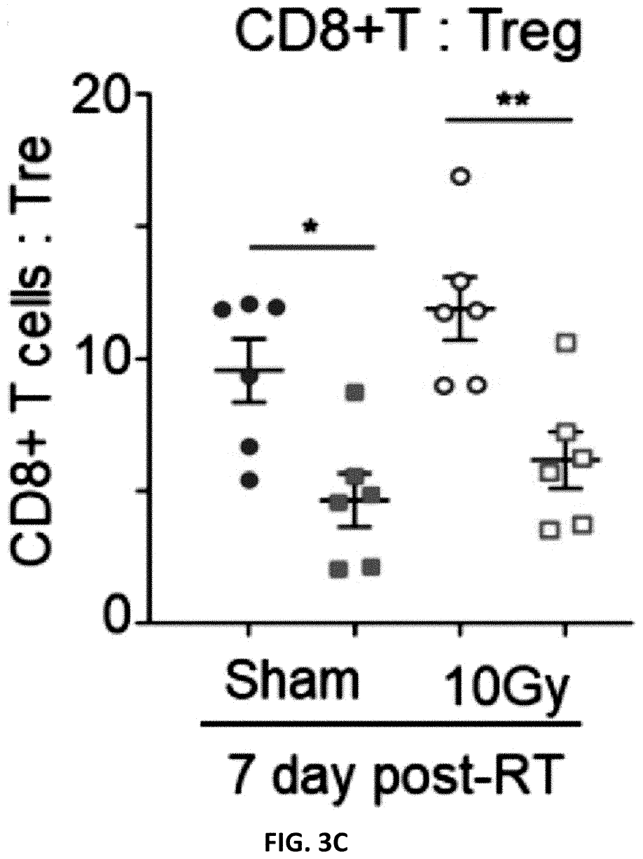

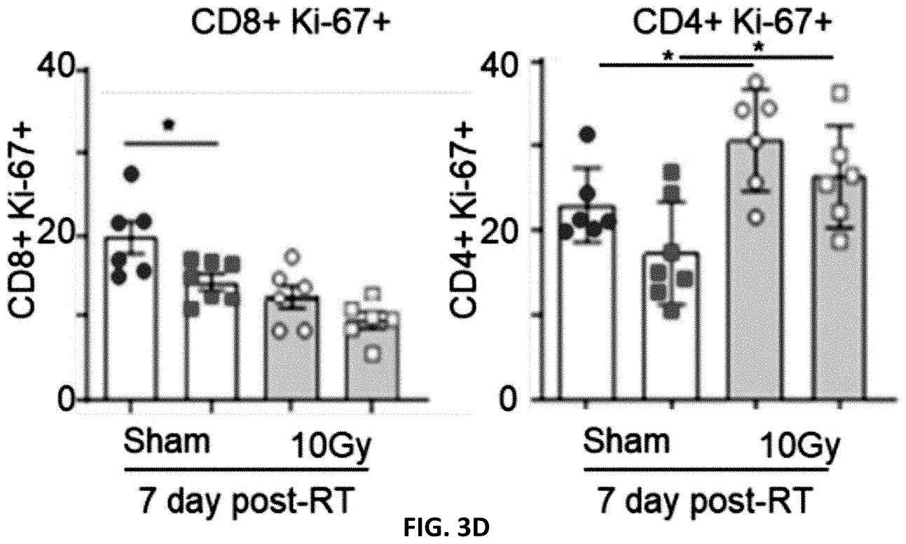

Two in vivo tumor models recapitulated these findings. First, athymic nude mice, receptive to human tumor cells but void of an intact T cell compartment, were injected with human cervix tumor cell lines engineered to express SERPINB3 (B3) or vector control (Ctrl), and mice were sacrificed at multiple time points. Second, lewis lung carcinoma (LL2) murine lung adenocarcinoma cells were engineered with the murine homolog mSerpinb3a (mB3a) or Ctrl and injected into immunocompetent C57BL6 mice. These mice were sacrificed at multiple time points before and after tumor-directed RT. In both tumor models, tumors expressing B3/mB3a demonstrated increased infiltration of TAMs and MDSCs (FIGS. 3A and B). In the LL2 model, mB3a-expressing tumors displayed a lower baseline CD8+:Treg ratio (FIG. 3C), lower T cell proliferation (FIG. 3D), and functional markers (TNFa+/IFNy+, FIG. 3E) following ex-vivo stimulation compared to Ctrl tumors. Whereas tumor-directed radiation of Ctrl tumors induced robust CD8+ T cell infiltration at 7-days post-RT, most of which had a non-Treg phenotype, mB3a tumors had minimal T cell infiltration, a low CD8+:Treg ratio, and a functionally exhausted phenotype as evidenced by low TNFa+/IFNy+ CD4+ and CD8+ T cells (FIG. 3C-E).

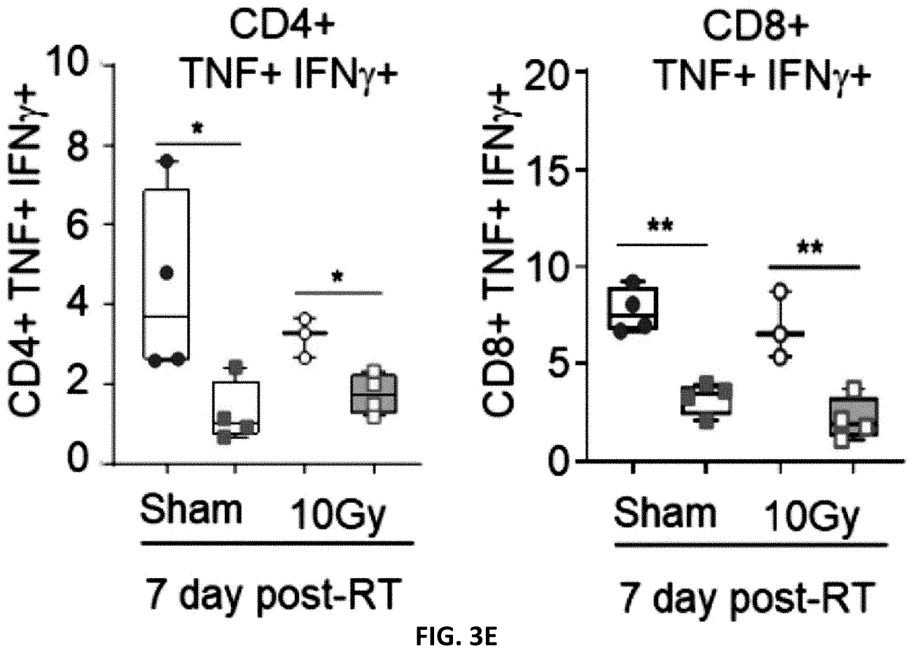

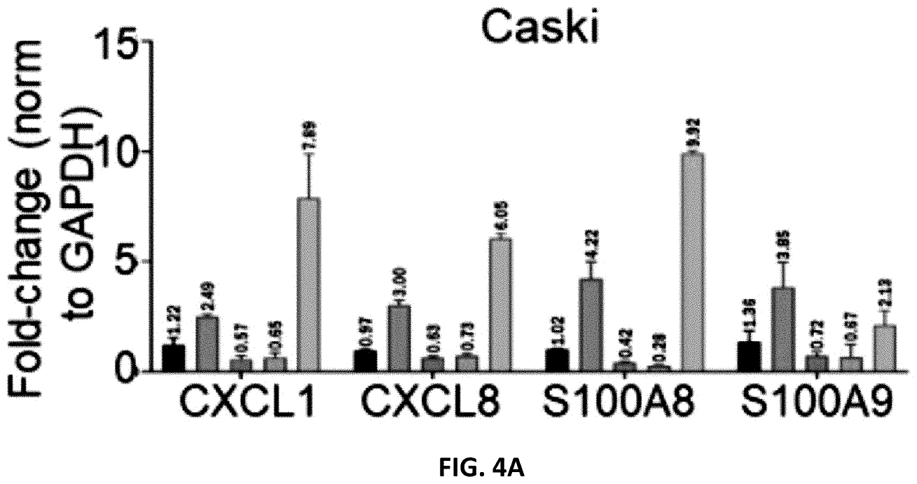

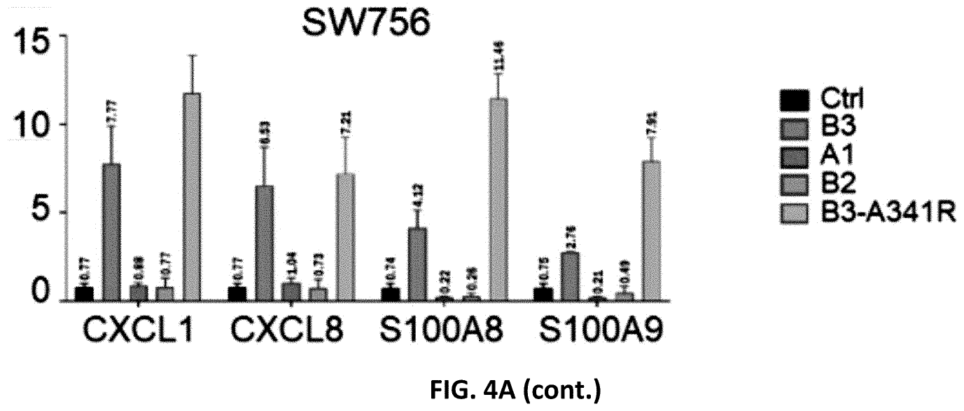

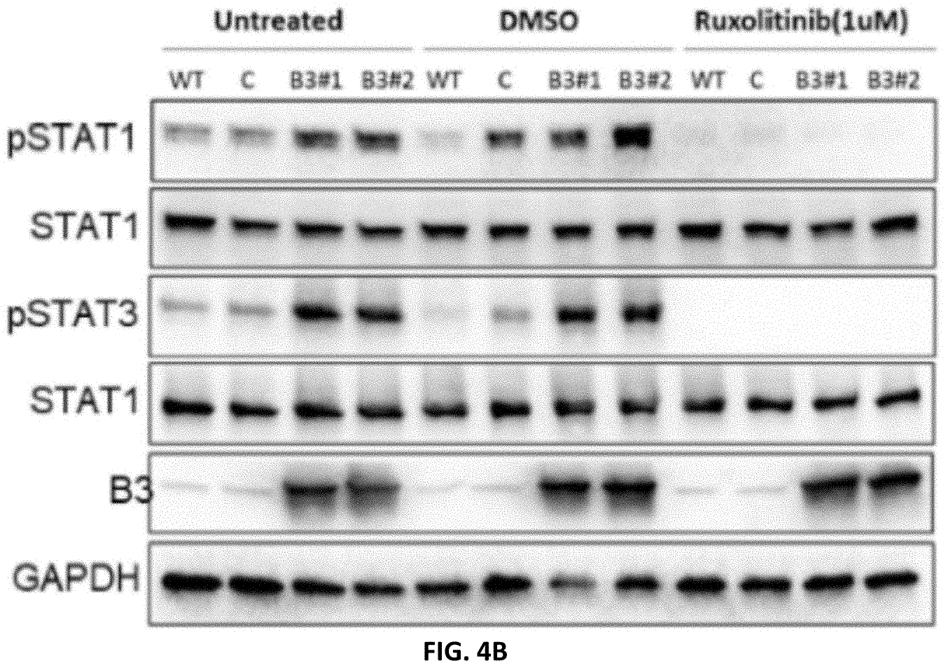

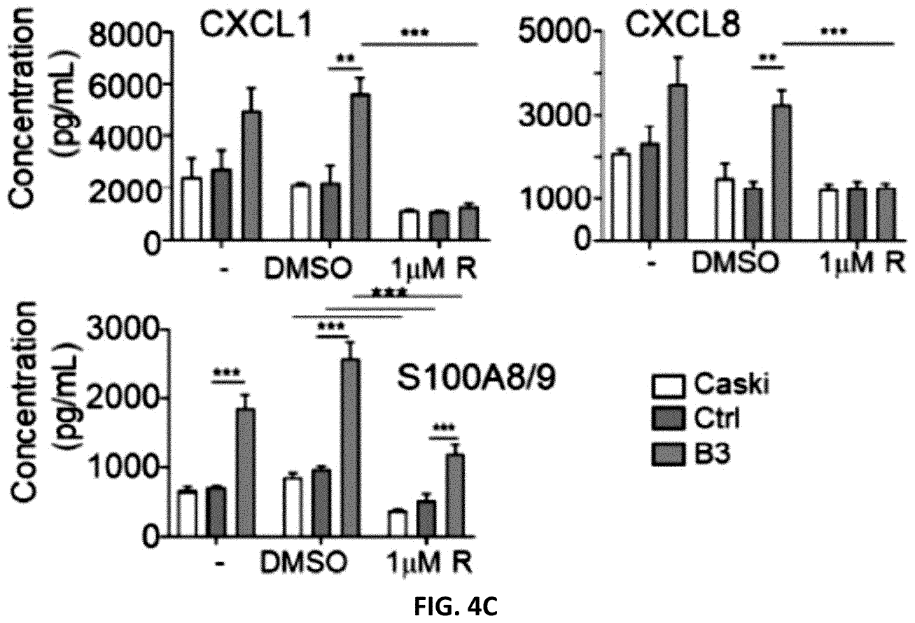

This preliminary data suggest that the alanine to arginine mutation at amino acid 341 within the RSL of SERPINB313, increased CXCL 1/8 and S100A8/9 expression, similar to wild-type B3, while expression of SERPINB2, or SERPINA 1 did not (FIG. 4A), suggesting a B3-specific, but RSL-independent mechanism.

To understand the molecular mechanism behind SERPINB3-mediated chemokine induction resulting in the reshaping of the tumor immune milieu, a kinase array was performed assaying five key signaling pathways involved in inflammation and immune response. STAT and MAPK family members were activated in B3 cells compared to Ctrl, confirmed on Western blot analysis (FIG. 4B). Among the NF-κB activation pathway factors, only upstream kinases MSK1 and TBK1 were more highly phosphorylated in B3 tumor cells, but not IκBα or p65. To determine if STAT activity was required for B3-dependent chemokine expression, cells were treated with ruxolitinib, a JAK1/2 inhibitor, which abolished phosphorylation of STAT1 and STAT3 (FIG. 4B), and prevented downstream B3-associated chemokine expression (FIG. 4C).

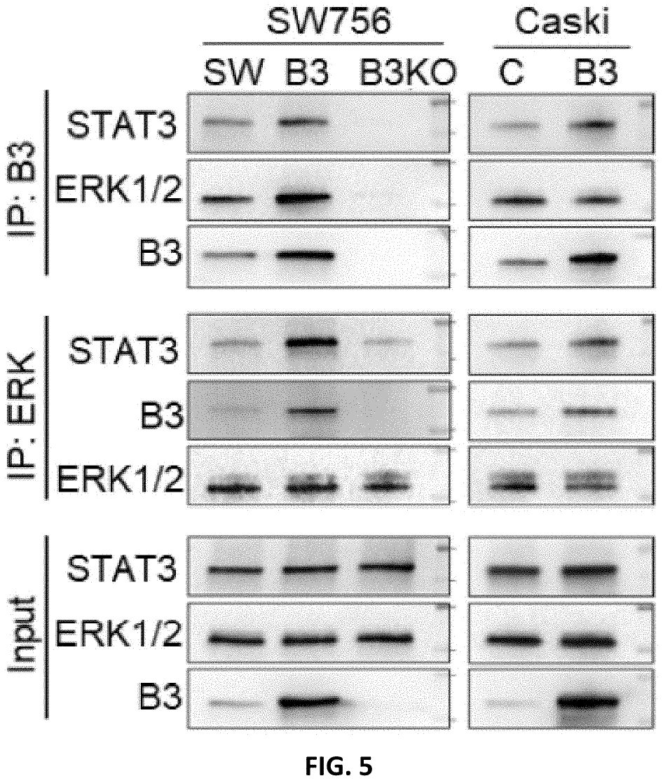

The most well-characterized molecular function of SERPINB3 is its role as an inhibitor of cysteine proteases including lysosomal cathepsins. As there is no known role for SERPINB3 target proteases in the regulation of these kinase signaling pathways or chemokine expression, a proximity labeling screen was performed with the high-efficiency biotinylation enzyme TurboID fused to the SERPINB3 sequence. After a short incubation with exogenous biotin, cells expressing B3-TurboID or TurboID alone were lysed, and streptavidin-enriched proteins were submitted for mass-spec analysis. Compared to TurboID alone, B3-TurboID biotinylated several proteins along the MAPK and JAK/STAT activation pathways, including STATS, MAPK3 (ERK1), and MAPK1 (ERK2). Co-immunoprecipitation with anti-SERPINB3 antibody demonstrated interaction with STAT3, and ERK1/2 (FIG. 5). While SERPINB3 has been shown capable of binding directly to JNK1 using in vitro crystal structure analysis, JNK1 co-immunoprecipitated with anti-SERPINB3 antibody was not observed.

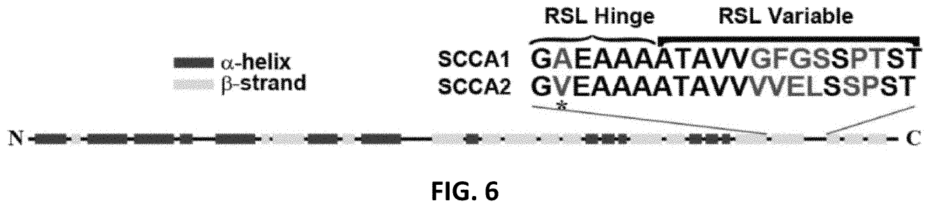

To determine if the reactive site loop of SERPINB3 is required for the upregulation of myeloid recruiting chemokines, the high degree of homology between SERPINB3 and SERPINB4, and the requirement for a specific RSL sequence to maintain specific protease inhibitory activity, is exploited. First, isogenic SERPINB3-KO cell lines genetically rescued with one of three inserts downstream of a human ubiquitin-promoter constitutive expression vector are used: 1) SERPINB3-wild type (B3 wt), 2) SERPINB3-A341R (83A341 R), and 3) SERPINB4 (B4), which shares 92% identity with SERPINB3 at the amino acid level and differs primarily at the RSL (FIG. 6). Clonal cell lines expressing comparable levels of these target proteins are analyzed by qRT-PCR and ELISA for expression and secretion of myeloid attracting chemokines CXCL 1, CXCL8, S100A8, and S100A9.

Co-IP of STAT3 and ERK1/2 with SERPINB3 (B3 wt and 83A341 R) or SERPINB4 (B4) antibodies are performed to determine if i) the reactive site loop of SERPIN83 is required for this interaction, and ii) if B4 is also interacting with or in complex with these kinases when expressed in cervix cancer cells. If analysis of these initial rescue lines suggests that the B3 RSL is not required for the interaction of SERPINB3 with STAT and ERK, or for chemokine induction, the KO lines will be rescued with serial truncation domain mutants of SERPINB3 to identify the minimal domain(s) required for co-IP and/or chemokine induction.

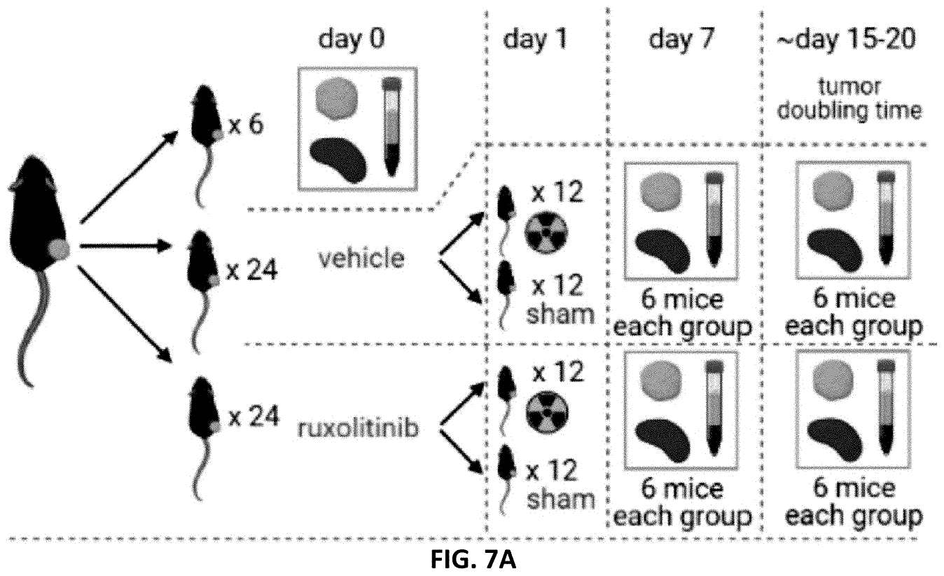

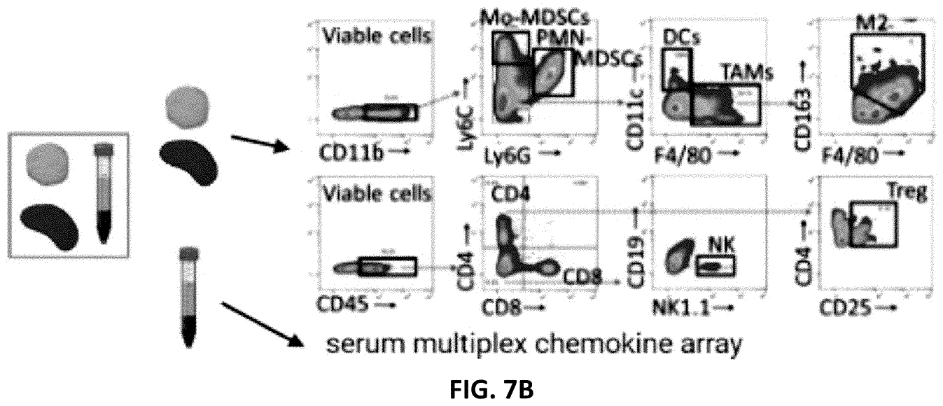

LL2 cells engineered to express mSerpinB3a are injected into the flank of C57BL6 mice. Tumor growth is monitored every 3-4 days and volume is estimated as V=0.5*(L×W″²), where V=volume, L=length of the long axis, and W=width of the short axis. When tumors reach ˜1 cm³, mice are randomized into five cohorts (FIG. 7A). One cohort is sacrificed without treatment, and tumors, spleens, and peripheral blood are harvested to determine baseline levels of intratumoral and systemic immune cells and chemokines as described in FIG. 7B. A second cohort is treated with vehicle control (20% Captisol in 58 mM citrate buffer, weight/volume) or 60 mg/kg ruxolitinib twice daily by oral gavage, followed by sham or 10Gy×2 using a tumor-directed approach. Half of each cohort is sacrificed at 7 days post-RT to determine radiation- and ruxolitinib-induced tumor infiltrating and systemic immune components. TDT is calculated as follows: TDT=ln 2/% change tumor volume per day.

### Example 2—SCCA1/SerpinB3 Promotes Suppressive Immune Environment Via STAT-Dependent Chemokine Production, Blunting the Therapy-Induced T Cell Responses

### Abstract

Cancer patients with high serum squamous cell carcinoma antigen (SCCA1/SERPINB3) are commonly associated with treatment resistance and poor prognosis. Despite being a clinical biomarker, the modulation of SERPINB3 in tumor immunity is poorly understood. Positive correlations of SERPINB3 with CXCL1/8, S100A8/A9, and myeloid cell infiltration were found through RNAseq analysis of human primary cervix tumors. Induction of SERPINB3 resulted in increased CXCL1/8 and S100A8/A9, which promoted monocyte and MDSC migration in vitro. In mouse models, Serpinb3a-tumors showed increased MDSC and TAM infiltration contributing to T cell inhibition and this was further augmented upon radiation. Intratumoral knockdown of Serpinb3a demonstrated tumor growth inhibition and reduced CXCL1, S100A8/A9, MDSC, and M2 macrophage infiltration. These changes led to enhanced cytotoxic T cell function and sensitized tumors to radiotherapy. It was further revealed SERPINB3 promoted STAT-dependent suppressive chemokine expression, whereby inhibiting STAT activation by ruxolitinib or siRNA abrogated CXCL1/8 and S100A8/A9 in SERPINB3 cells. Patients with elevated pre-treatment SCCA and high pSTAT3 had increased intratumoral CD11b+ myeloid cells compared to patients with low SCCA and pSTAT3 cohort that had overall improved survival after radiotherapy. These findings provide a preclinical rationale for targeting SERPINB3 in tumors to counteract the immunosuppression and improve response to radiation.

### Introduction

Radiotherapy (RT) is commonly used in the treatment of patients with squamous cell carcinomas, including head and neck, esophageal, lung, and cervical cancers. RT can have both immunostimulatory and immunosuppressive effects, which in part influence the prognosis of cancer. The activation and infiltration of cytotoxic T cells post-radiation is critical to the curative activity of RT. However, tumors with an immunosuppressive tumor microenvironment (TME), dominated by myeloid cells, such as M2 macrophage polarization, myeloid-derived suppressor cell (MDSC), tend to diminish T cell activity and may be more susceptible to the suppressive immune response induced by RT. Chemokines are a subclass of cytokines with chemotactic properties that control the migration of cells and influence the composition of the tumor immune microenvironment. Some chemokines promote an immunostimulatory environment, such as CXCL9, CXCL10, CXCL11, and CXCL16, which improve dendritic cell activation and T cell trafficking to tumors. Conversely, CCL2, CCL5, CXCL1, CXCL8, and CXCL12 can be induced by RT and have the opposite effect of recruiting suppressive immune cells, inhibiting effector T cells, and are often correlated with poor treatment outcomes.

Squamous cell carcinoma antigen 1 (SCCA), encoded by the SERPINB3 gene locus and now known as SERPINB3, is a highly conserved cysteine proteinase inhibitor that interacts with lysosomal proteases upon lysosomal leakage and prevents cell death. It has recently been demonstrated that SERPINB3 also protected cervix tumor cells against RT-induced cell death by preventing lysoptosis. In many cancers, SERPINB3/SCCA1 was highly expressed in tumors or in the circulation of cancer patients, including cervical, head and neck, lung, breast, and esophageal cancers, often associated with poor prognosis, treatment outcomes, and recurrence. In addition, elevated SERPINB3 expression was also found in autoimmune disorders and implicated in the induction of inflammatory cytokines. However, in both tumors and autoimmune diseases, the mechanistic link between SERPINB3 and immune regulation remains poorly understood. Considering the increasing number of studies reporting the association of SERPINB3 with tumorigenesis, metastasis, prognosis, and recurrence, additional roles of SERPINB3, independent of proteinase-inhibitory activity, in tumor progression and resistance to therapy are likely.

It was previously demonstrated that patients with persistently high levels of SCCA before treatment and throughout the course of definitive RT had an increased risk of recurrence and death. Prospective cohort studies also showed the prognostic value of SCCA for monitoring the response to RT and clinical outcome post-RT in cervical cancer patients. Given the unfavorable outcomes of patients with high SERPINB3 expression, it was hypothesized that SERPINB3 promotes immune evasion by modulating suppressive immune responses that alter the tumor microenvironment and impede RT-induced anti-tumor immunity. The data showed that SERPINB3-expressing tumors secreted high levels of chemokines that attract myeloid cells. These myeloid cell populations in SERPINB3 tumors possessed potent immunosuppressive activity and inhibited T cell activation, leading to a resistant environment to RT. Targeting CD11b+ myeloid cells or SERPINB3 both reduced tumor growth, however, the latter in combination with RT demonstrated more sustained inhibition of tumor growth and remodeling of infiltrating myeloid cells. It was further discovered that STAT signaling plays an essential role in inducing suppressive chemokine expression in SERPINB3-expressing cells. Cervical cancer patients with high SERPINB3/SCCA were associated with increased pSTAT3 and CD11b expression. Here, a regulatory function of SERPINB3 in establishing a pro-tumor microenvironment and the clinical importance of targeting SERPINB3 to improve RT-induced antitumor immunity is presented.

### Results

### SERPINB3 Tumors are Marked by Myeloid Cell-Rich and Suppressive Immune Profile

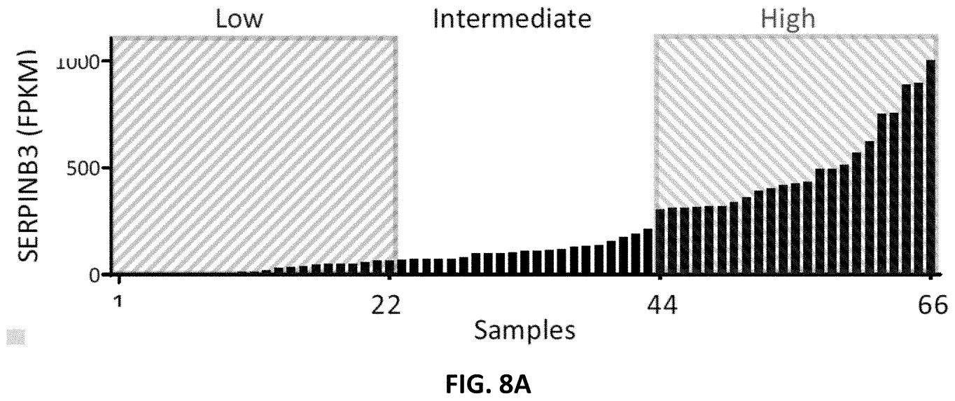

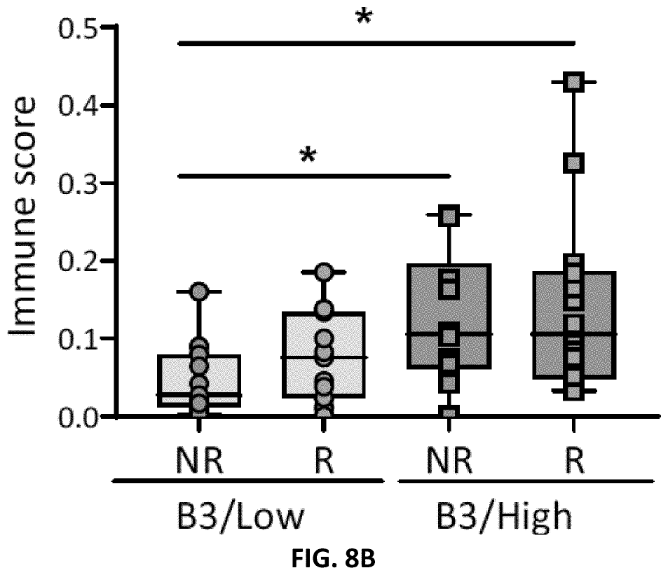

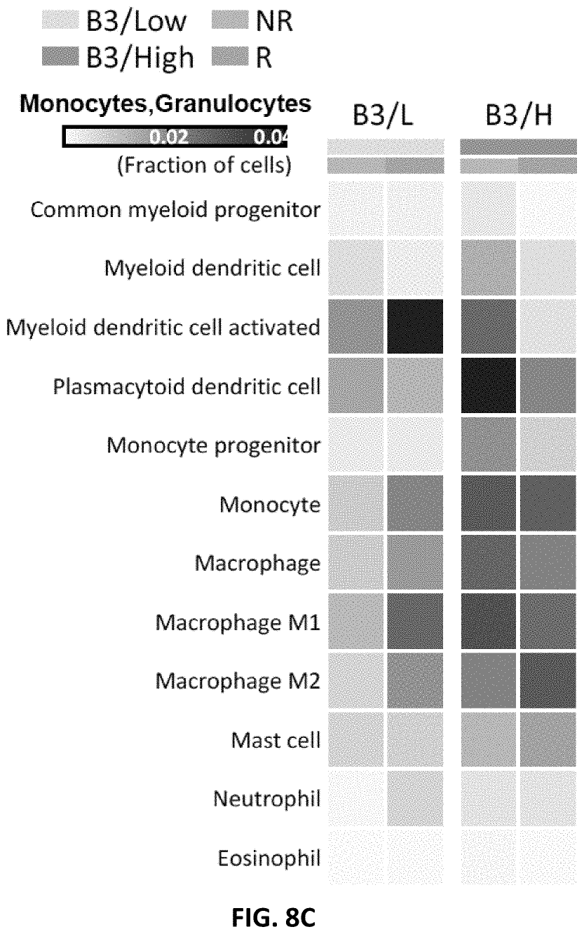

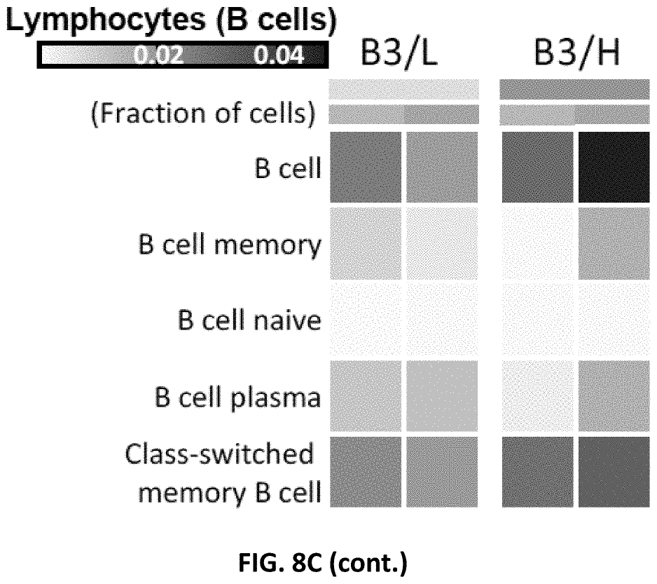

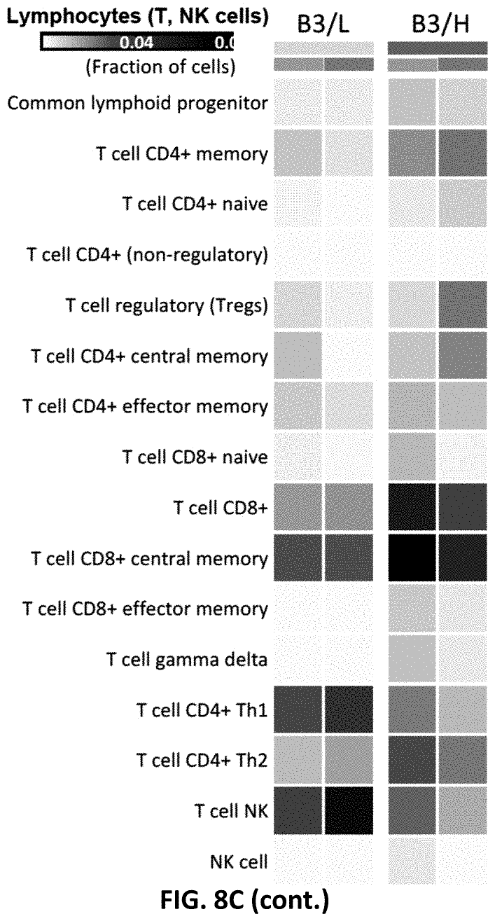

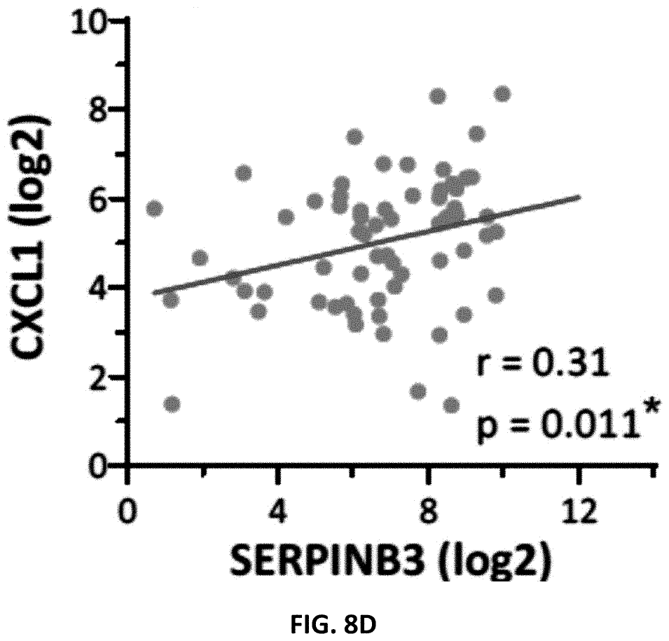

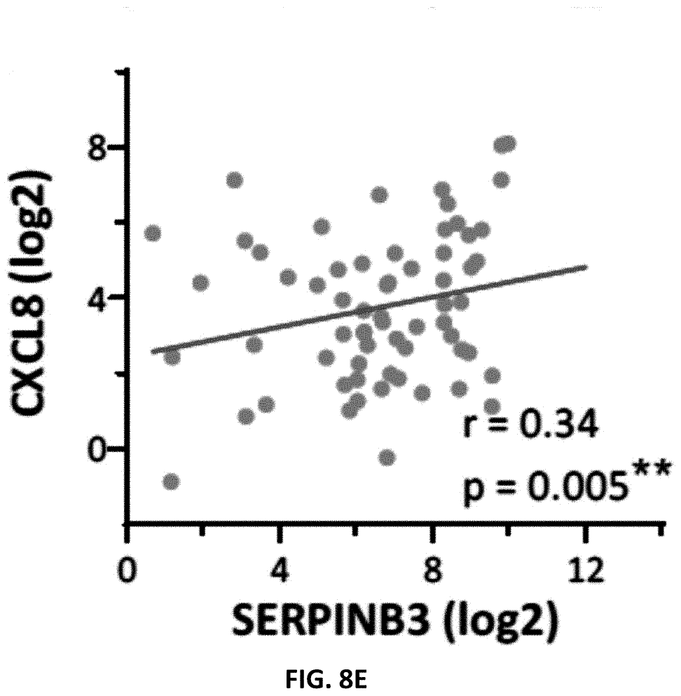

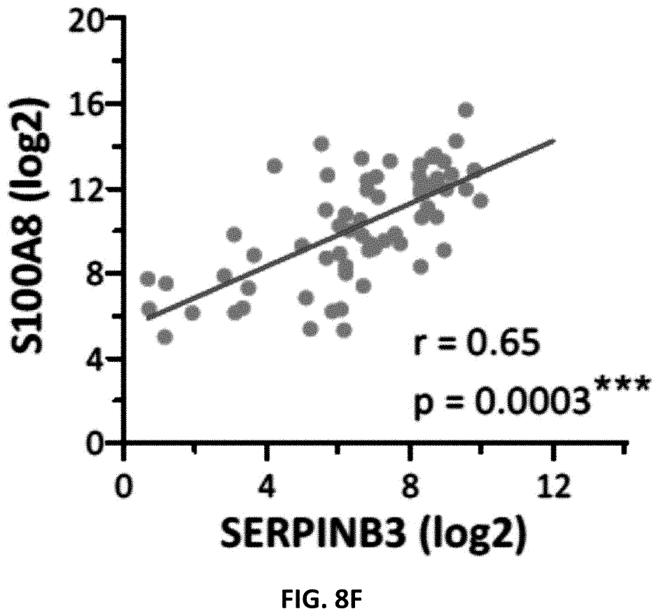

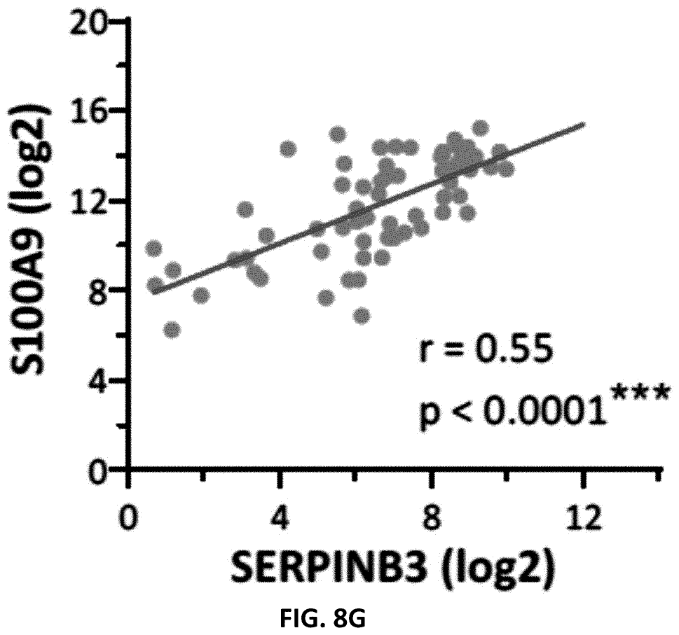

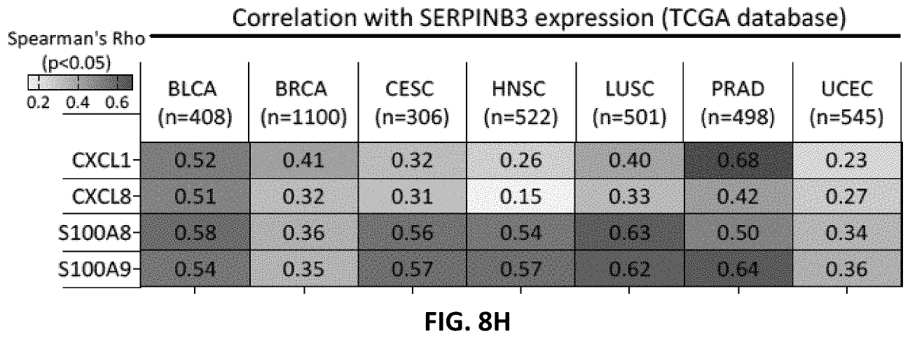

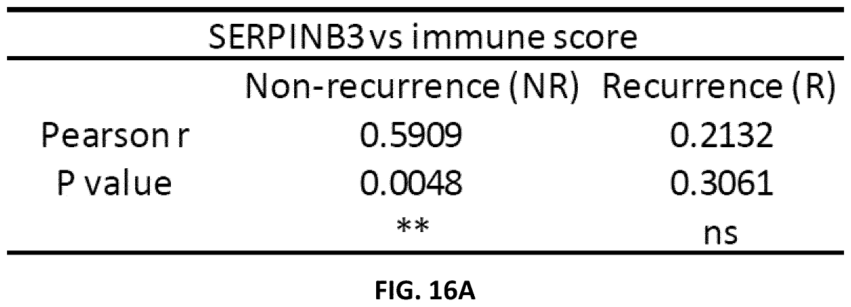

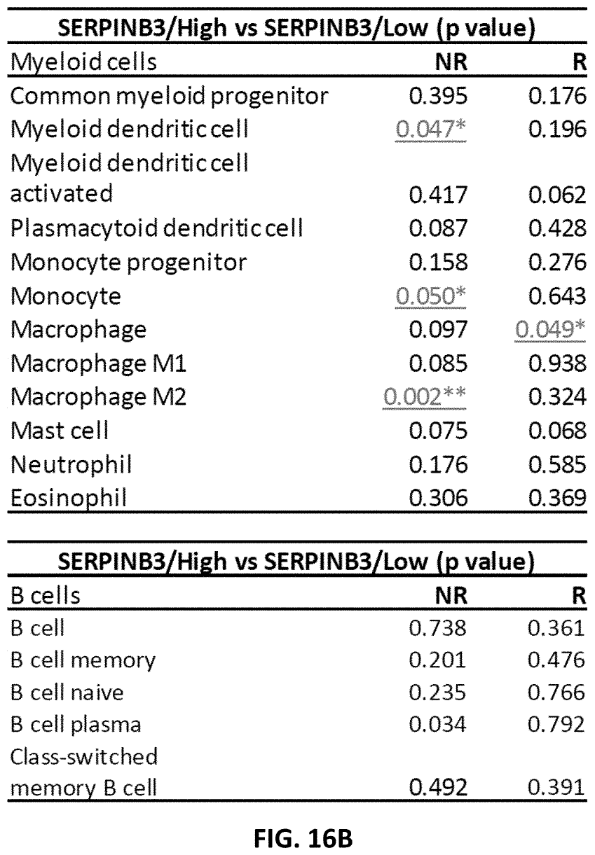

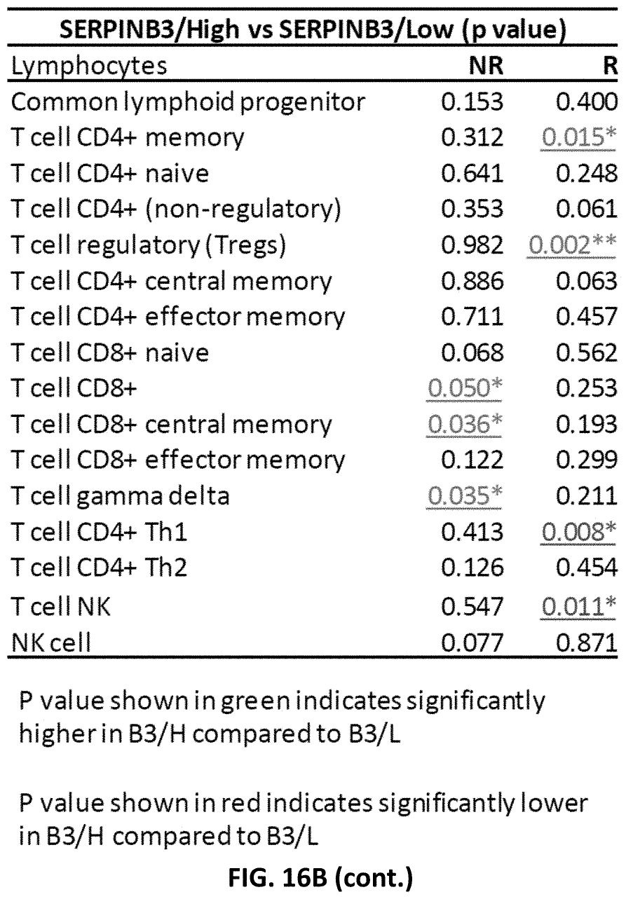

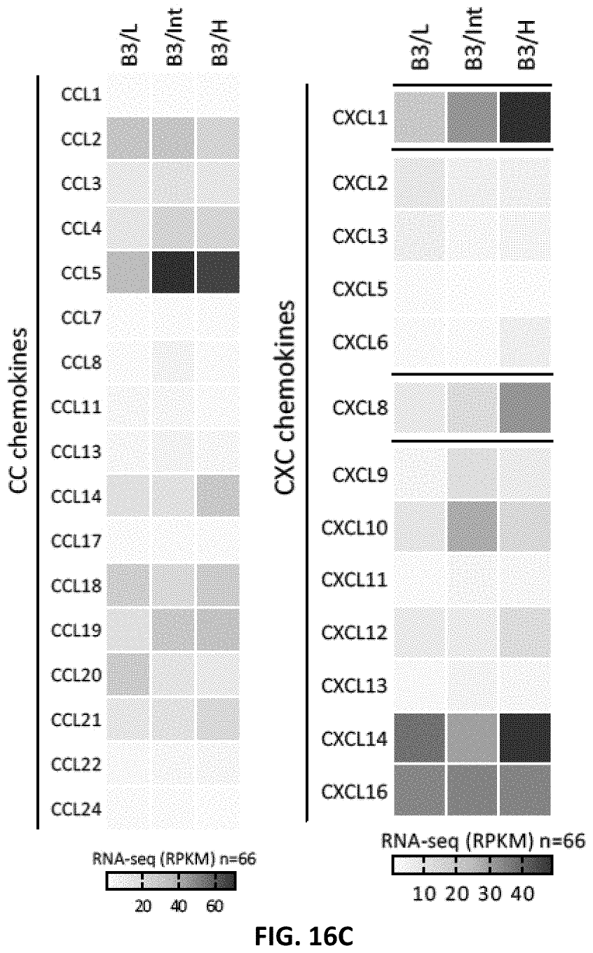

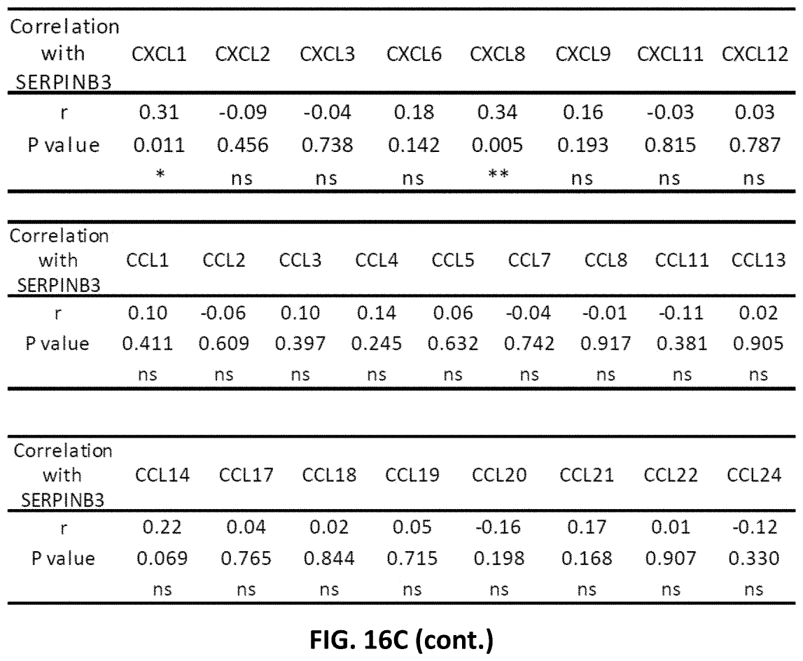

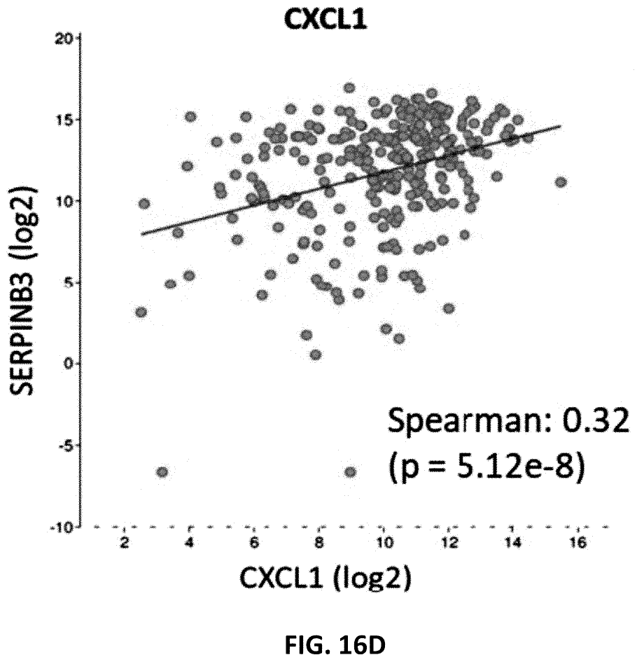

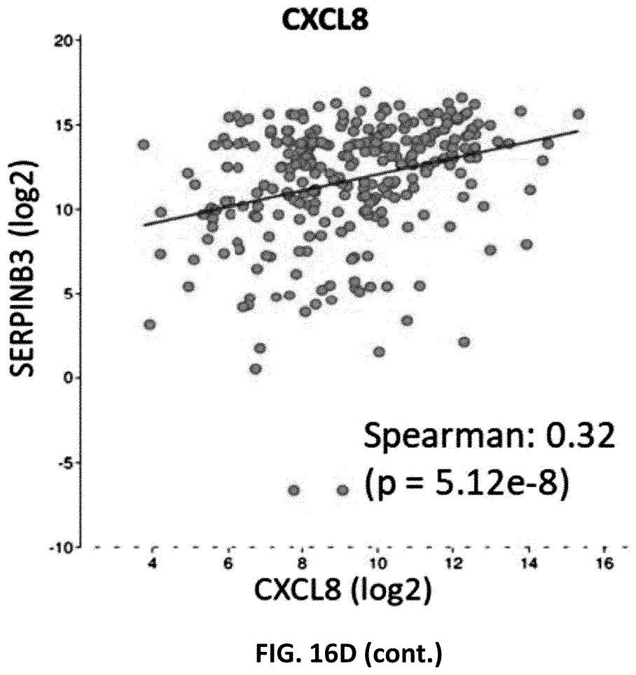

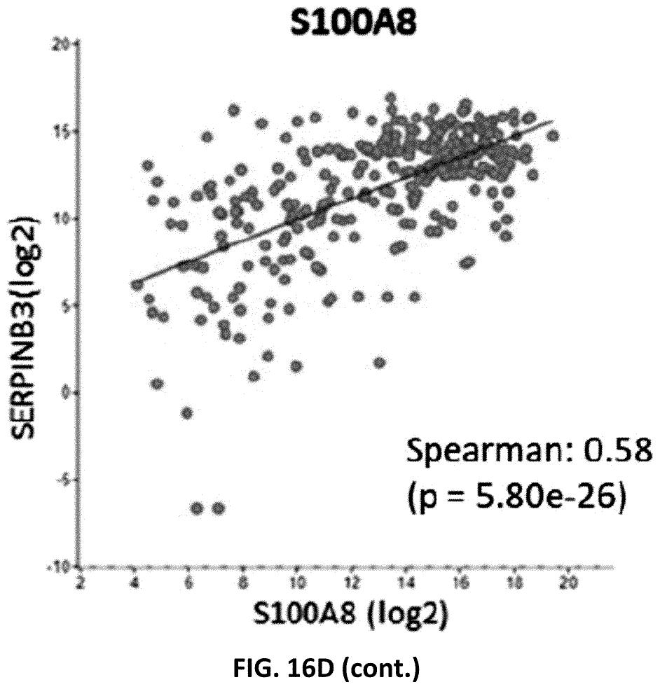

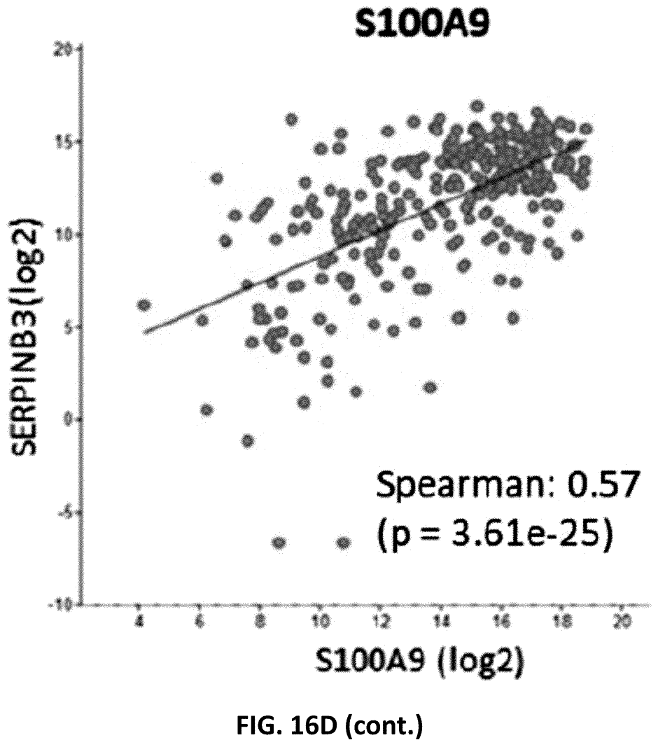

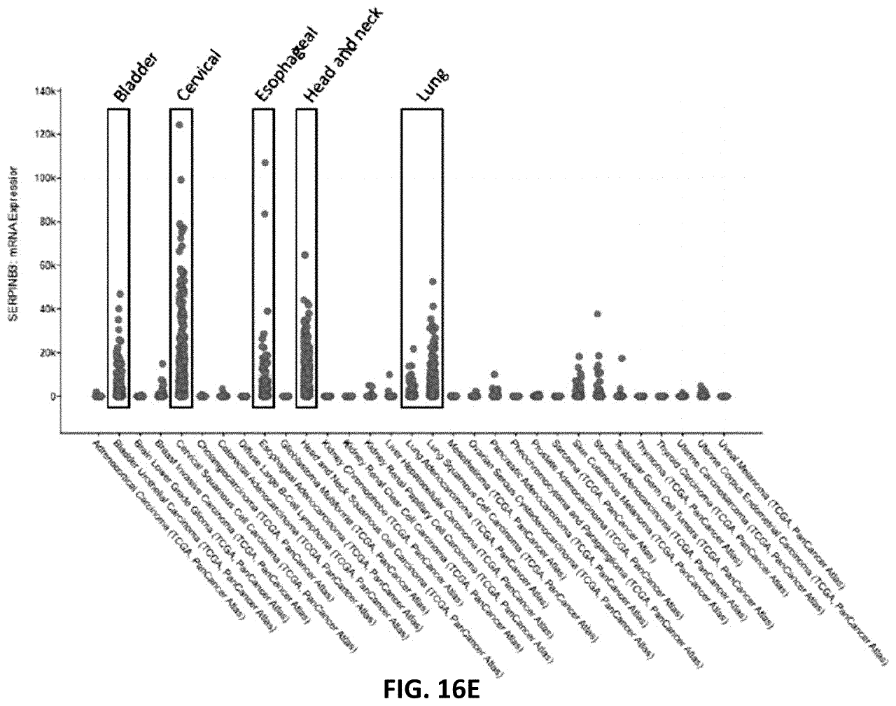

RNAseq was performed on 66 cervical tumor biopsies collected prior to (chemo)-RT. Patient and tumor characteristics of this cohort are summarized in FIG. 26. Patients were divided into three groups based on the distribution of SERPINB3 transcript levels; SERPINB3-low (B3/L, n=22), SERPINB3-intermediate (B3/Int, n=22), and SERPINB3-high (B3/H, n=22) groups (FIG. 8A). To investigate the distinct immune signature associated with SERPINB3 expression in tumors, analysis was focused on B3/L versus B3/H patient groups. The Immune Score (IS) was determined by xCell, via gene signature-based single-sample gene set enrichment analysis with the overall score representing a ranking of tumors in the dataset by lowest (IS of 0) to highest immune infiltrate. B3/H tumors from patients who eventually experienced recurrence (R) compared to those who remained recurrence-free (NR) showed overall higher immune scores compared to B3/L tumors indicating a potential immune-rich microenvironment (FIGS. 8B and 16A). Immune cell content showed that B3/H tumors were characterized by increased myeloid cell subsets, including macrophages, monocytes, plasmacytoid dendritic cells, and a small subset of CD8 T lymphocytes. In contrast, T-helper type 1 (Th1), Th2, and natural killer T cells were lower in B3/H compared to B3/L tumors (FIGS. 8C and 16B).

We then investigated the differential expression of two major human chemokine subfamilies, CC and CXC chemokines, in the three groups was then investigated (FIG. 16C). Two chemokines associated with the recruitment of myeloid cells, CXCL1 and CXCL8, correlated with SERPINB3 expression (FIGS. 8D and 8E). In contrast, expression of T cell- and NK cell-recruiting chemokines CXCL9, 10, and 16, were not associated with SERPINB3 expression (FIG. 16C). Further analysis of chemokines that attract myeloid cells demonstrated a positive correlation between SERPINB3 and S100A8/S100A9 expression (FIGS. 8F and G). These correlations were validated in the TCGA-CESC (cervical squamous cell carcinoma and endocervical adenocarcinoma, n=306) dataset (FIG. 16D). Notably, analysis of the TCGA PanCancer Atlas showed a consistent positive correlation between SERPINB3 and myeloid-attracting chemokine expression across multiple tumor types including bladder, breast, head and neck, lung, prostate and uterine cancers (FIG. 8H). These same tumor types have high levels of SERPINB3 expression (FIG. 16E).

SERPINB3 Results in Upregulation of CXCL1/8 and S100A8/A9 Chemoattractants, Promoting Myeloid Cell Migration from Patient-Derived Peripheral Blood

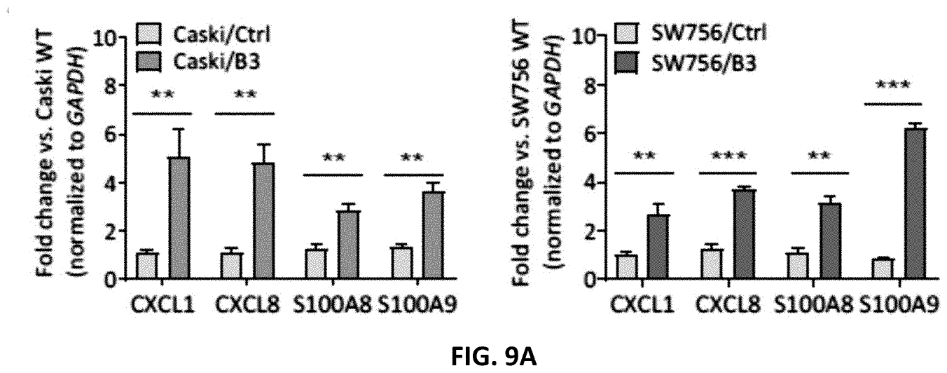

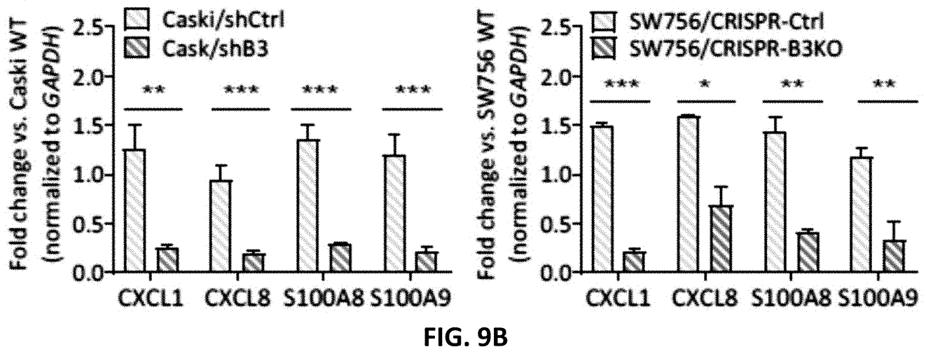

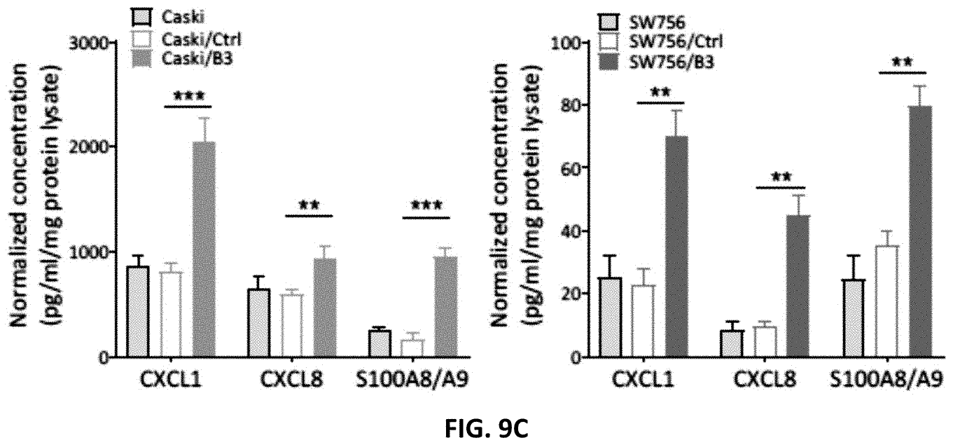

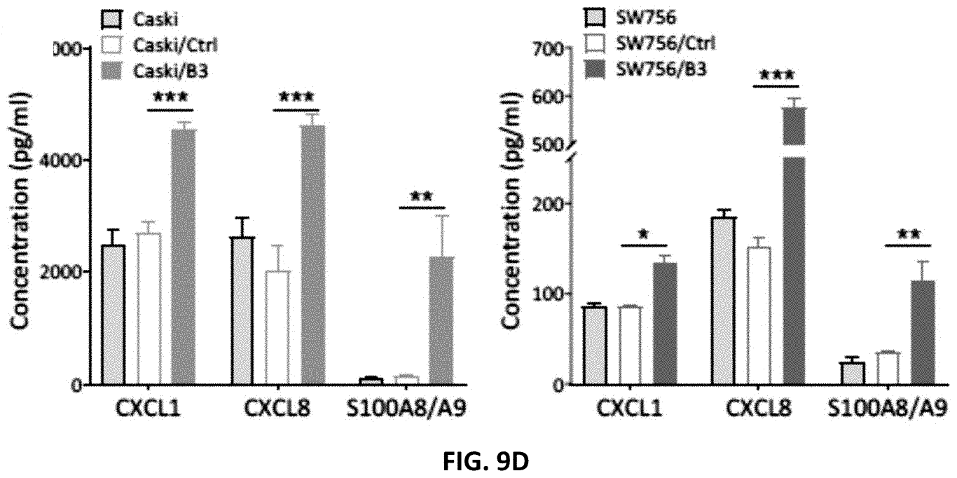

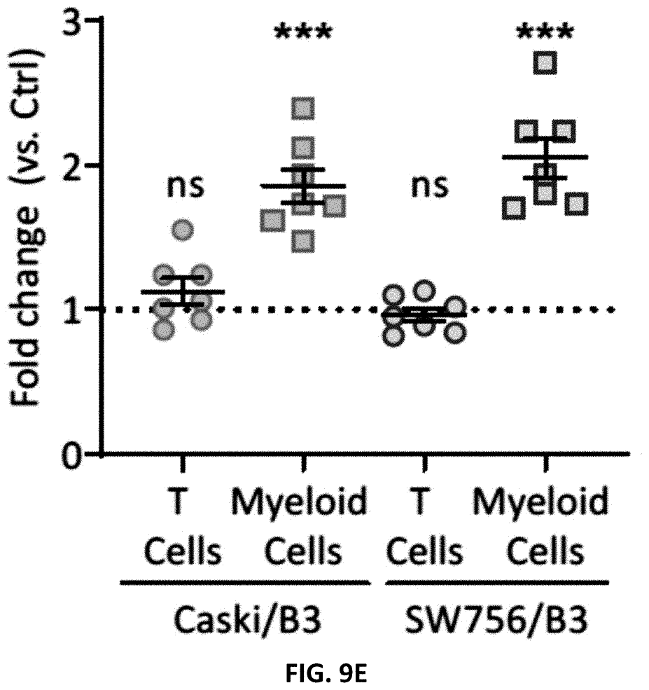

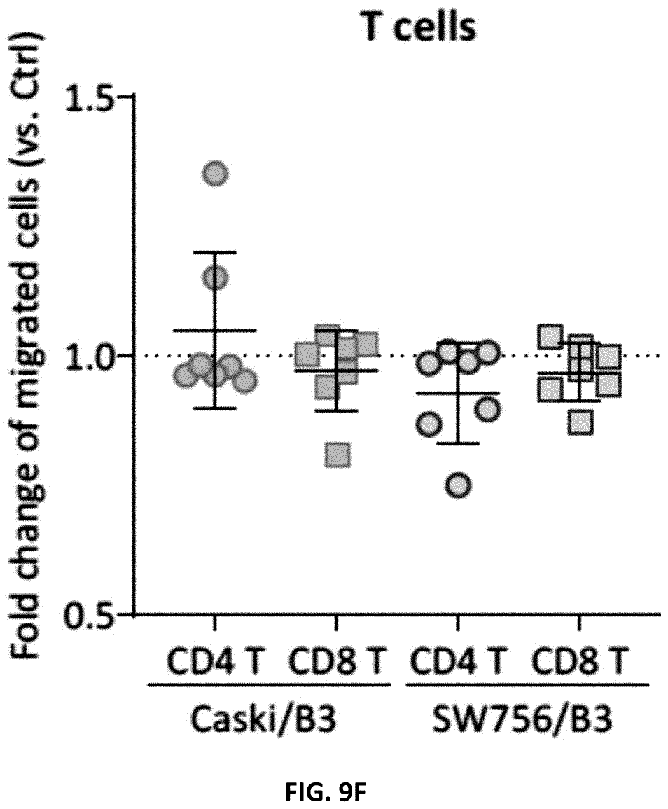

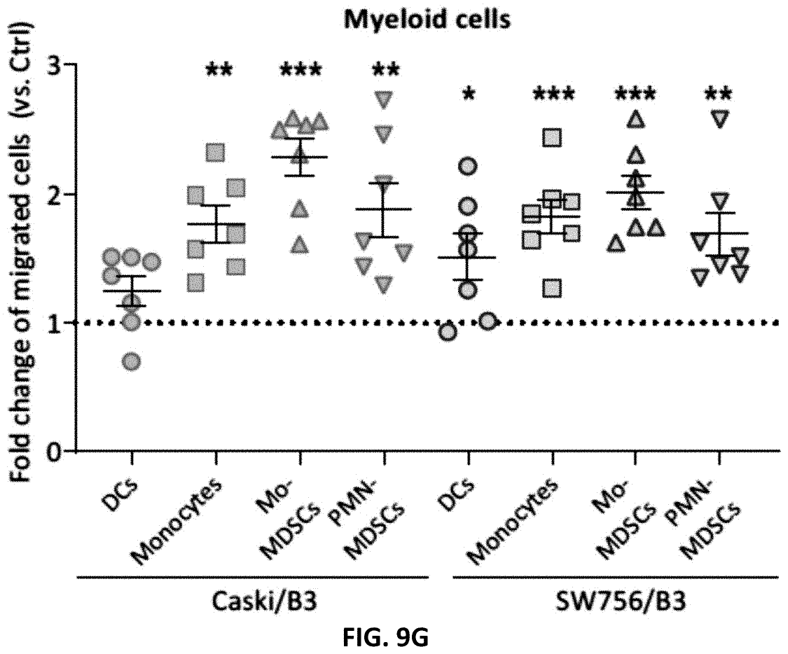

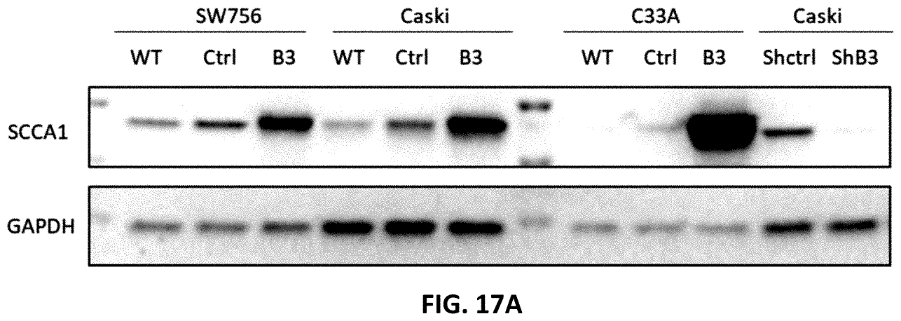

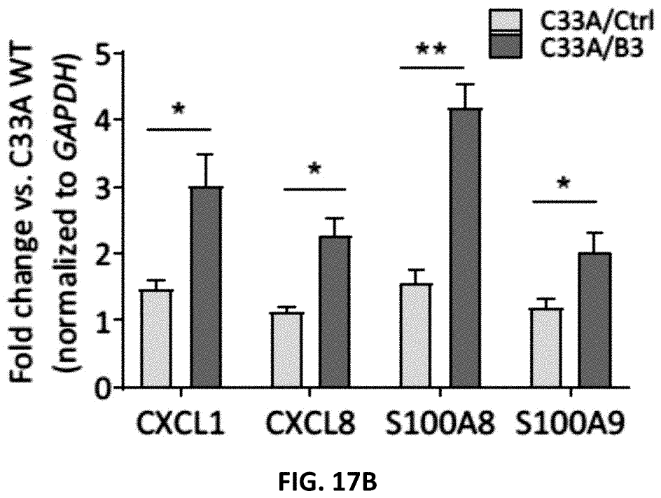

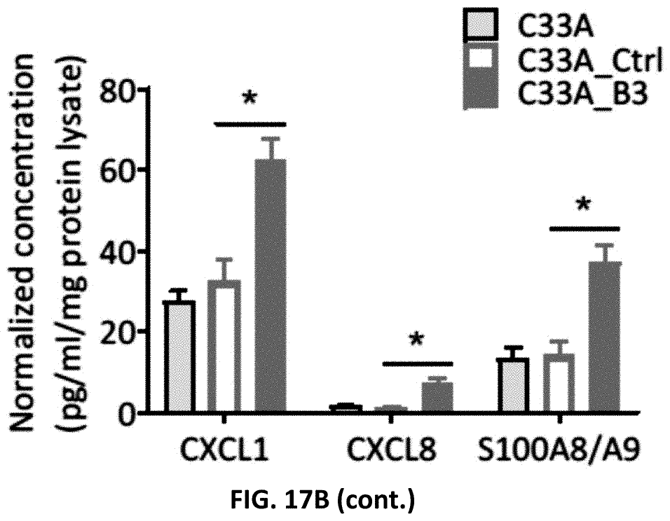

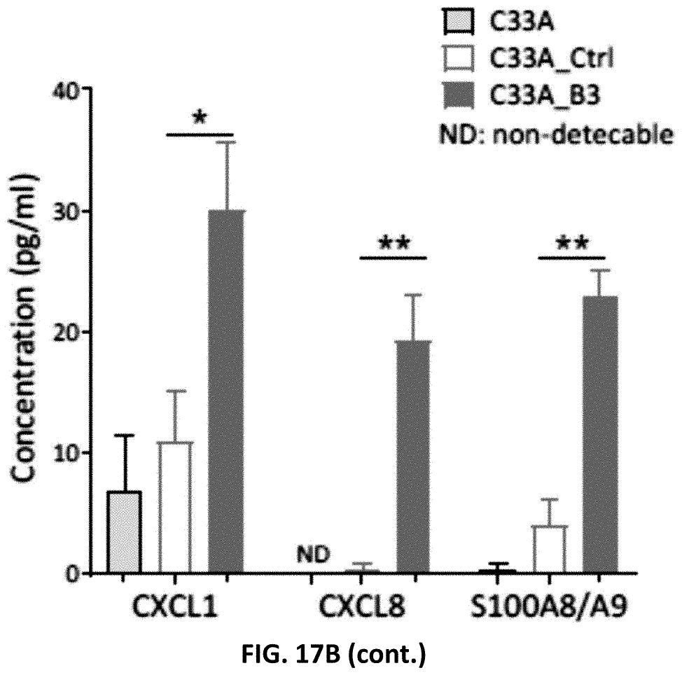

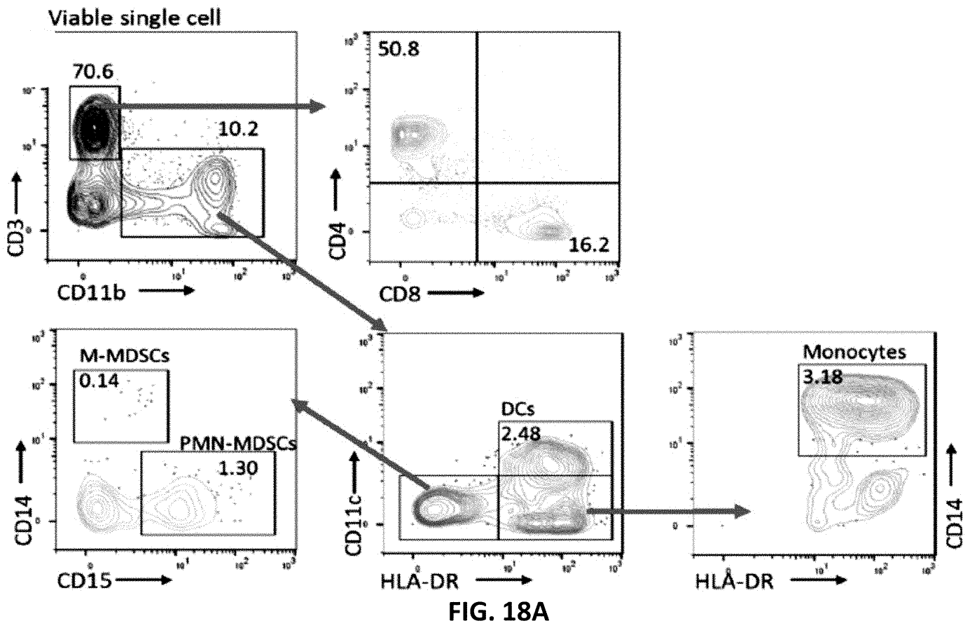

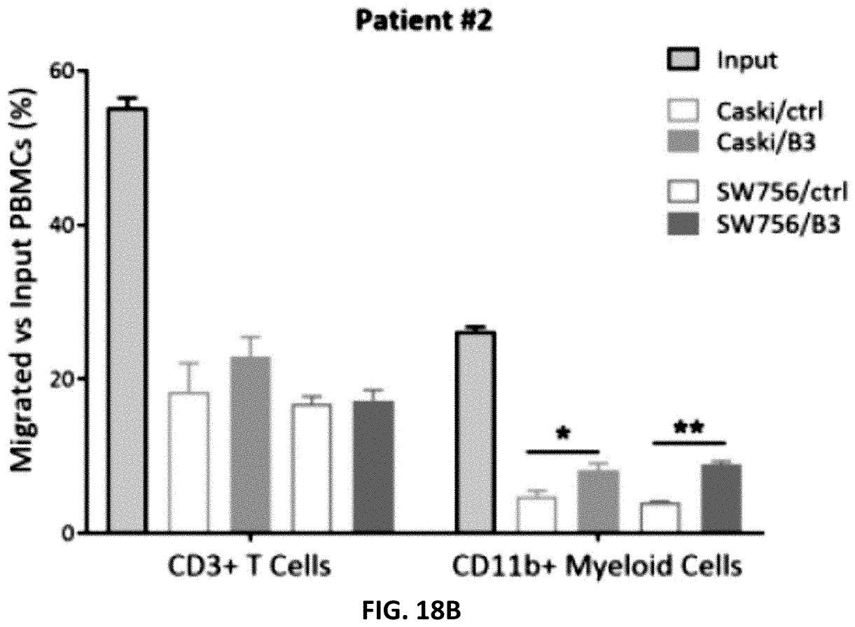

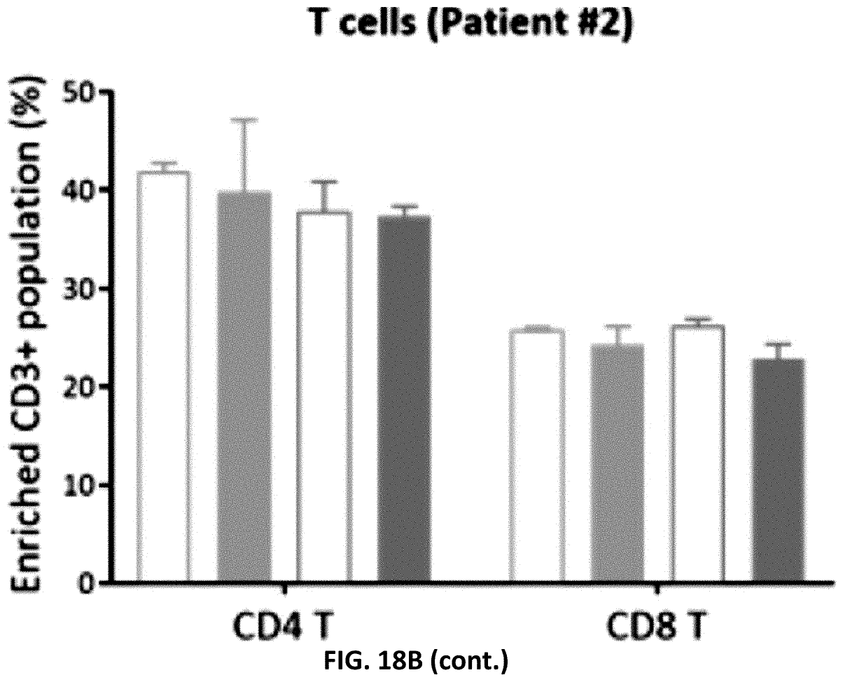

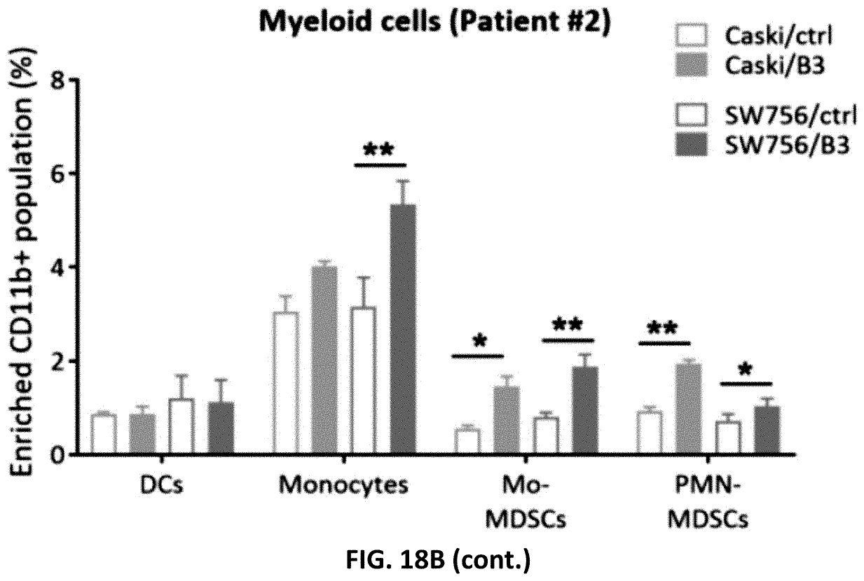

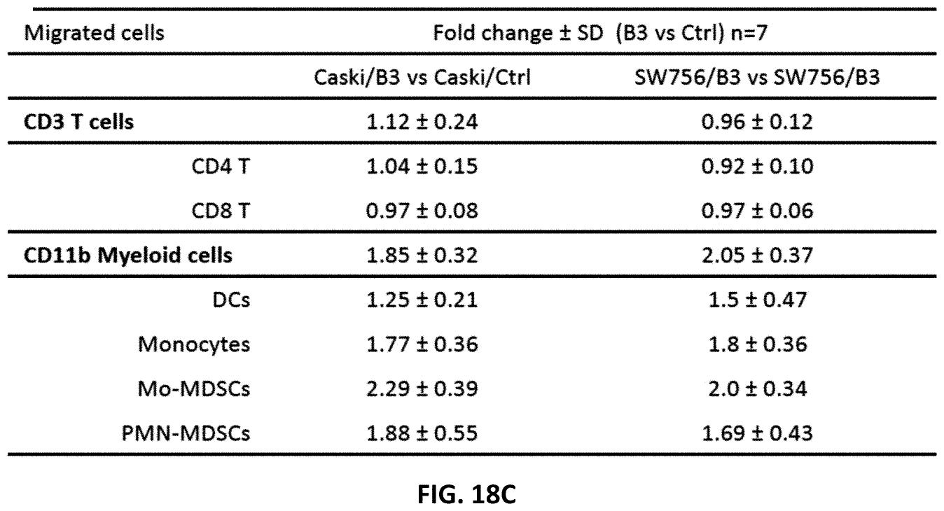

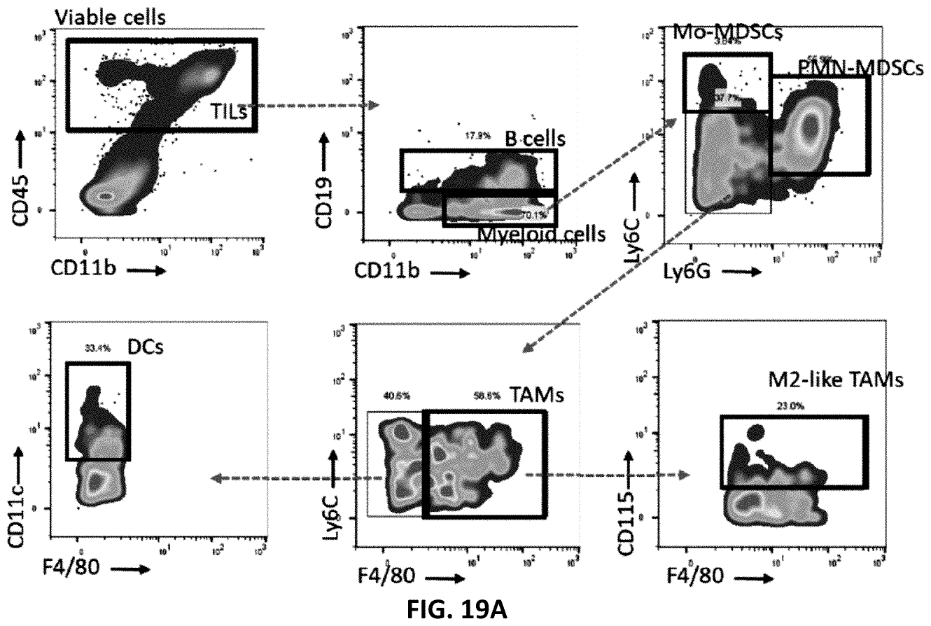

To study the mechanistic link between SERPINB3 and chemokine expression, SERPINB3 levels were genetically altered in human cervical cancer cells, Caski and SW756 cells (FIG. 17A), and examined the effect on chemokine production. Caski and SW756 with stable expression of SERPINB3 (Caski/B3, SW756/B3) showed increased CXCL1/8 and S100A8/A9 gene expression (FIG. 9A) while downregulating SERPINB3 using shRNA (Caski/shB3) or CRISPR-Cas9-mediated deletion (SW756/CRISPR-B3KO) significantly reduced CXCL1/8 and S100A8/A9 expression (FIG. 9B), when compared to their control counterparts. In addition to gene expression, significantly higher CXCL1/8 and S100A8/A9 protein expression and secretion were detected in Caski/B3 vs. Caski/Ctrl as well as SW756/B3 vs. SW756/Ctrl (FIGS. 9C and D). Because Caski and SW756 are positive for HPV16 and HPV18 respectively, whether SERPINB3-induced chemokine expression is associated with HPV infection was examined using HPV-negative cervical cancer cells, C33A. Similar to the observation in HPV-positive cells, C33A with SERPINB3 upregulation (C33A/B3) showed increased CXCL1/8 and S100A8/A9 expression, suggesting an HPV-independent mechanism (FIG. 17B). The chemotactic response of human peripheral blood mononuclear cells (PBMCs) was examined, obtained from seven patients with biopsy-proven cervical cancer prior to delivery of any treatment, to the chemokines secreted by tumor cells with high SERPINB3 expression. Supernatant collected from Caski/B3 and SW756/B3 promoted the migration of CD11b+ myeloid cells, with an average of 1.9-fold increase in Caski/B3 vs. Caski/Ctrl and a 2.1-fold increase in SW756/B3 vs. SW756/Ctrl, whereas the migration of CD4+ and CD8+ T cells showed no statistical difference (FIGS. 9E and F). Further analysis of migrated CD11b+ cells showed that populations migrating in response to both Caski/B3 and SW756/B3 supernatant were enriched in monocytes, and monocytic and polymorphonuclear myeloid-derived suppressor cells (M−/PMN− MDSCs) with an approximately 1.5-2 fold increase compared to Ctrl supernatant (FIGS. 9G, 18B, and 18C).

SERPINB3 Tumors Show Accumulated Myeloid Cells and Increased Tumor Growth

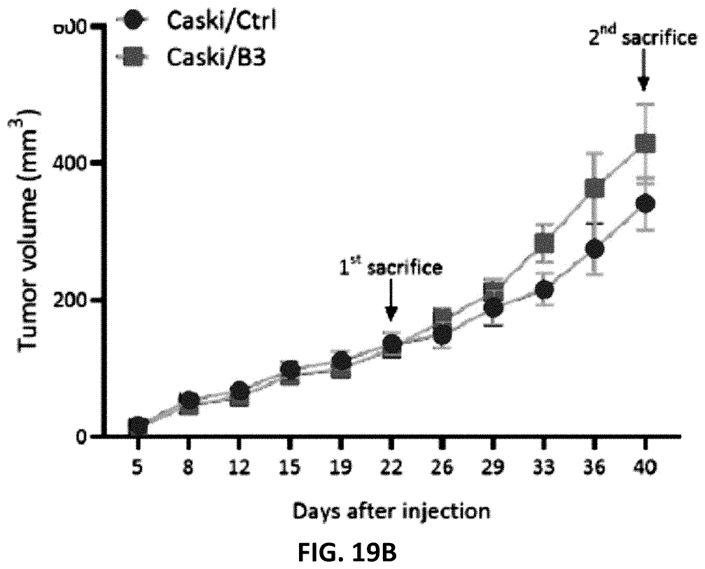

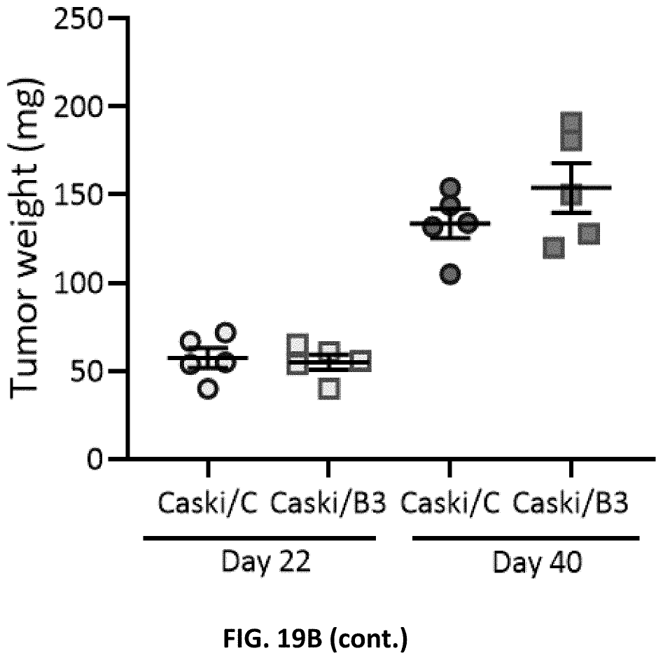

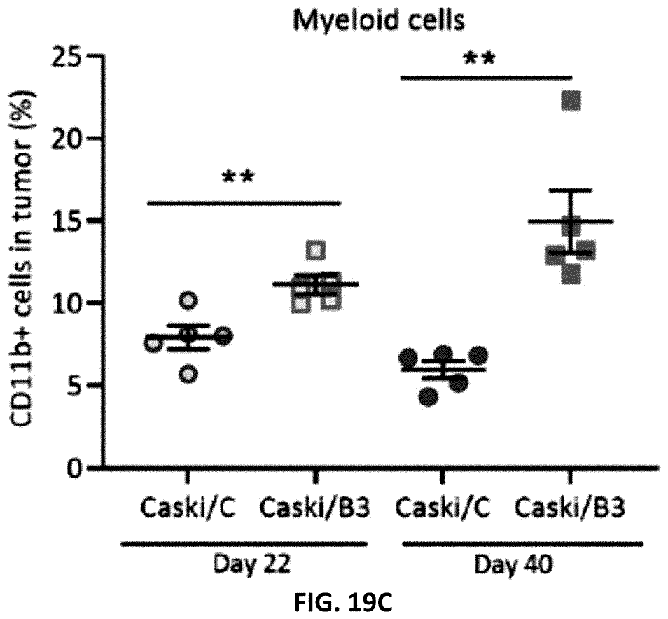

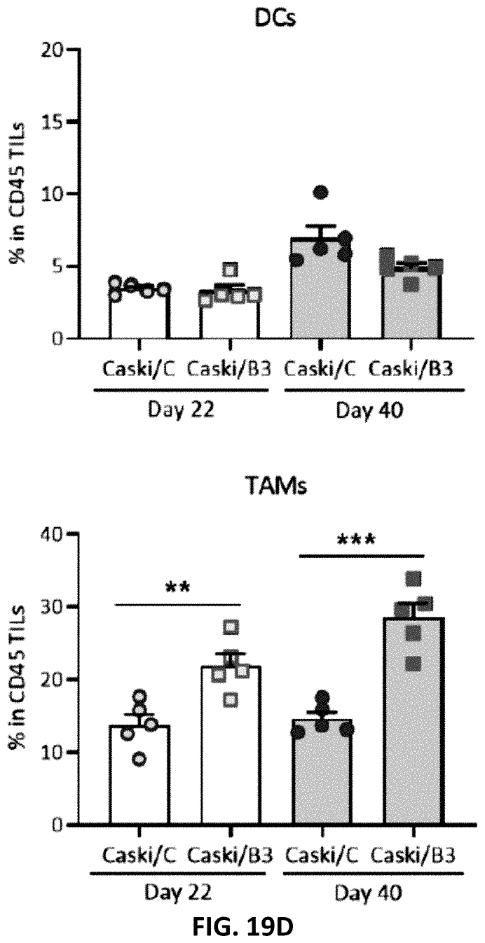

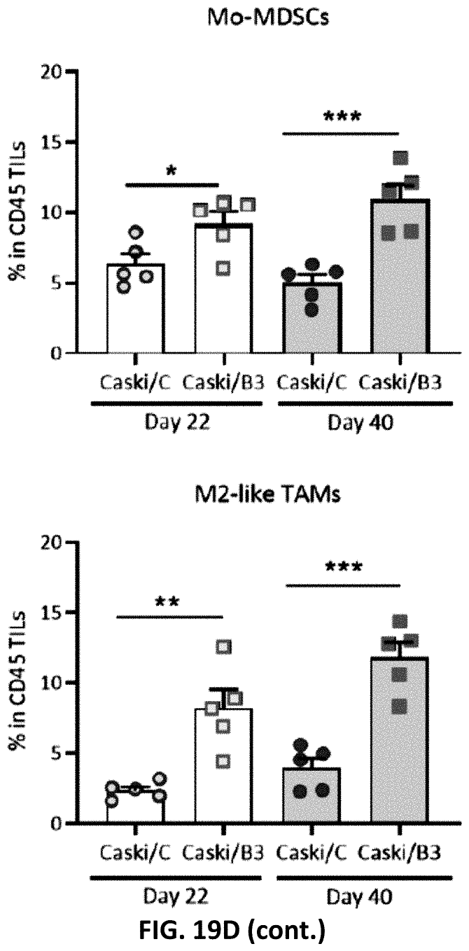

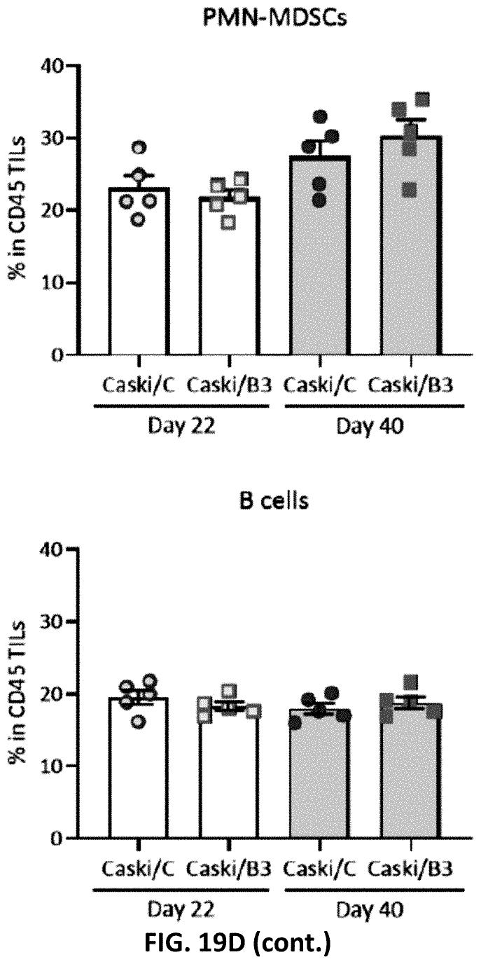

Since SERPINB3 upregulated the expression of myeloid chemoattractant in vitro, it was hypothesized that tumors expressing SERPINB3 attracted myeloid cell infiltration and mediate in vivo TME. Human Caski/Ctrl or Caski/B3 cells were injected subcutaneously on the flank of athymic nude mice and tumor-infiltrating myeloid cells were analyzed using flow cytometry (FIG. 19A). Tumor growth showed no difference between Caski/Ctrl and Caski/B3 over the course of the experiment (FIG. 19B); however, Caski/B3 tumors had a significant increase in infiltrating CD11b+ myeloid cells compared to Caski/Ctrl tumors at days 22 and 40 post-injection (FIG. 19C). M-MDSCs, TAMs, and M2 macrophages were significantly increased in Caski/B3 at both days 22 and 40, while no difference was seen in DCs, PMN-MDSCs, and B cells (FIG. 19D).

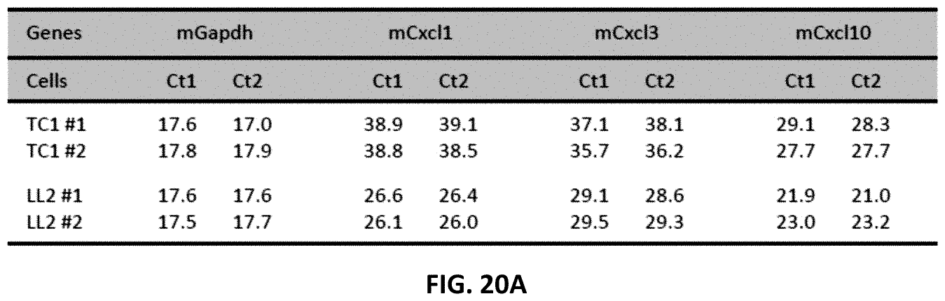

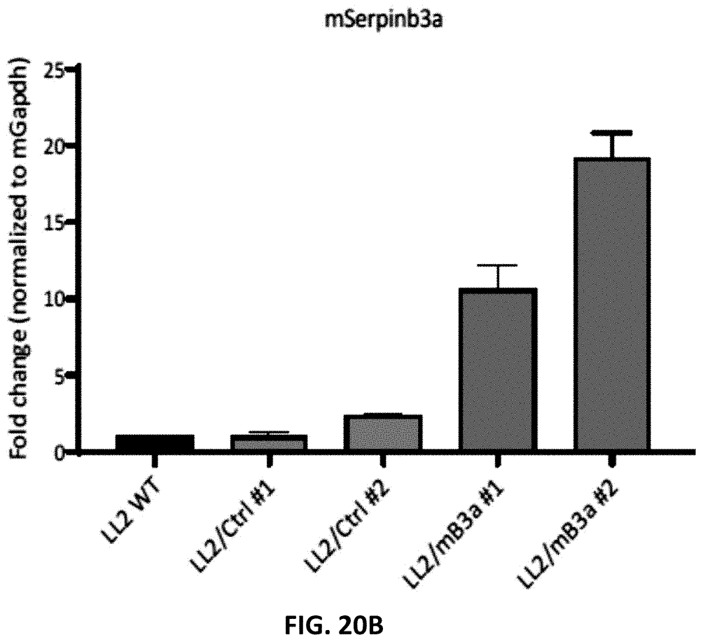

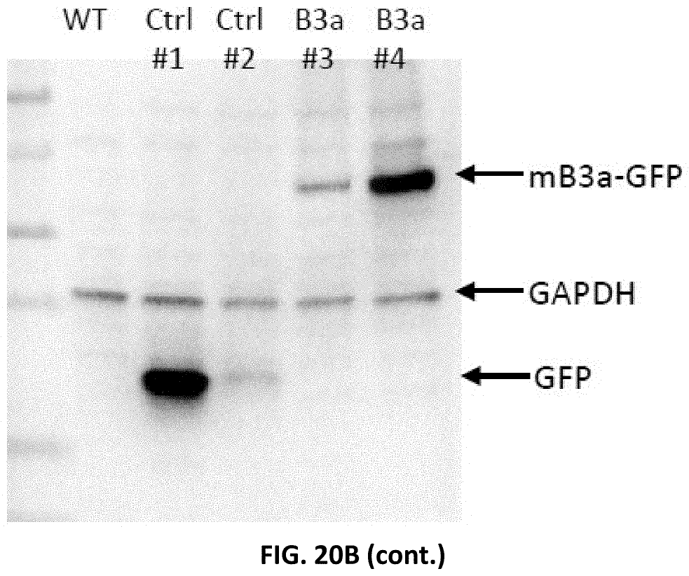

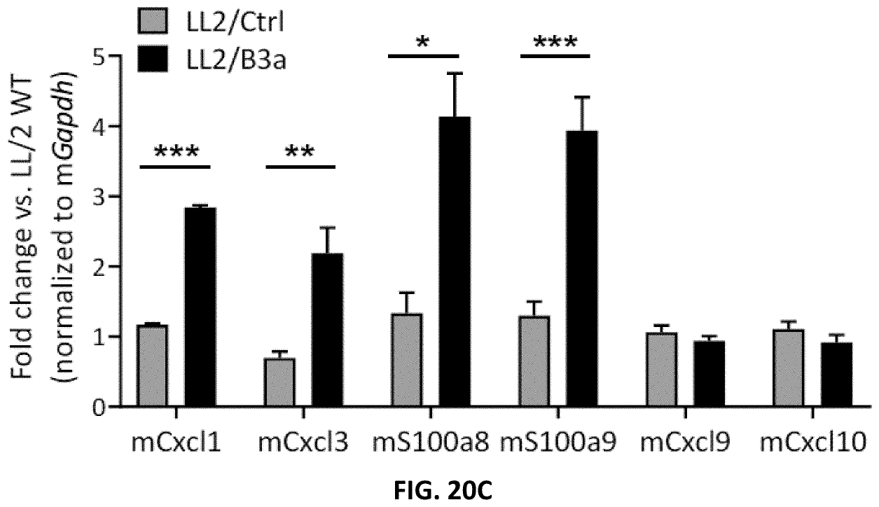

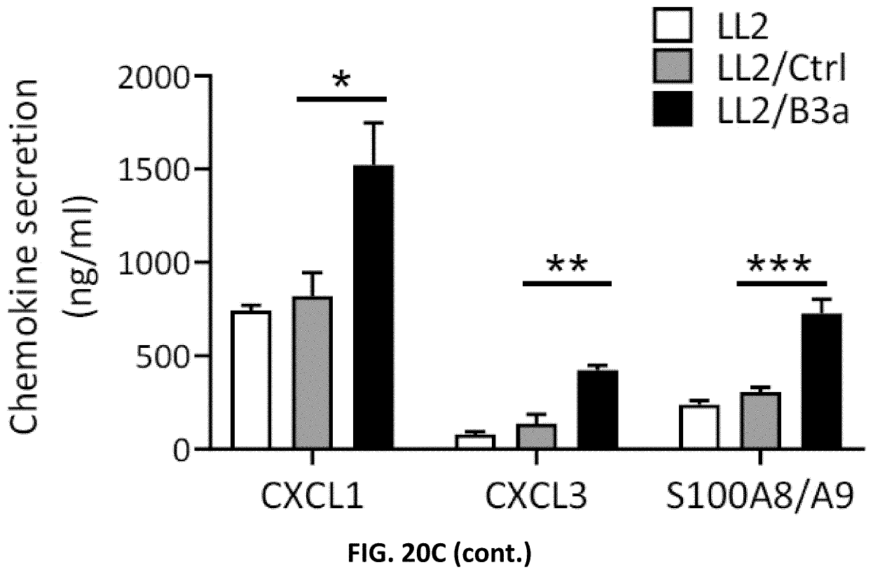

Given that lymphocyte-mediated immune activities play a role in tumor response to radiotherapy, and that RT is known to reshape the TME, SERPINB3-mediated TME and its response to radiation were characterized in an immunocompetent murine model. However, there are no murine cervical tumor lines, and the commonly used alternative, TC1 cells with HPV E6/E7 gene expression, were derived from normal lung epithelial cells with relatively low chemokine expression (FIG. 20A). Therefore, constructs driving murine Serpinb3a, homologous to human SERPINB3, were expressed in LL2 murine lung carcinoma cells (LL2/B3a) and an empty vector was used as a control (LL2/Ctrl) (FIG. 20B). Of note, SERPINB3 is also expressed in lung cancer (FIG. 16E) and negatively associated with prognosis, providing credence to this model.

As in the human cervical cancer model, the gene expression and secretion of murine Cxcl1/3, functionally corresponding to human CXCL1/8, and murine S100a8/9, homologous to human S100A8/9, were induced by mSerpinb3a, whereas chemokines associated with T cell migration, mCxcl9, and mCxcl10 were not affected by mSerpinb3a expression (FIG. 20C).

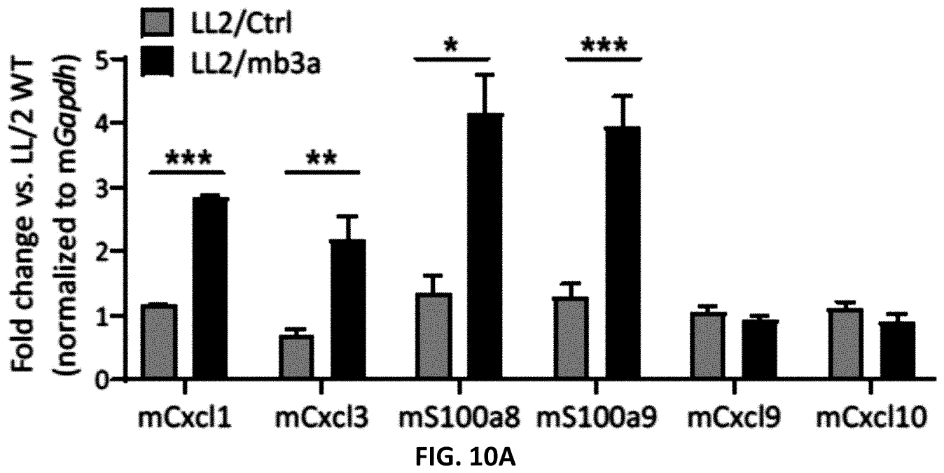

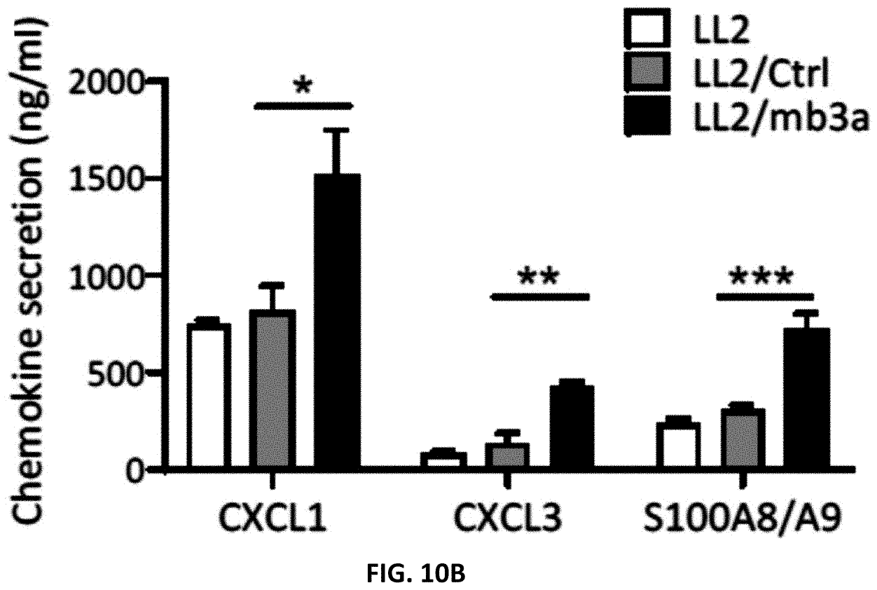

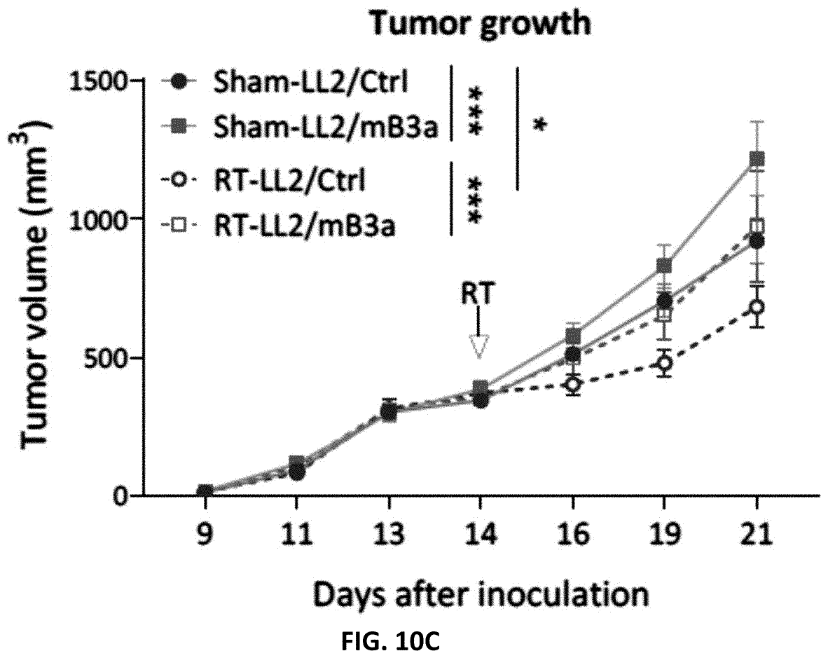

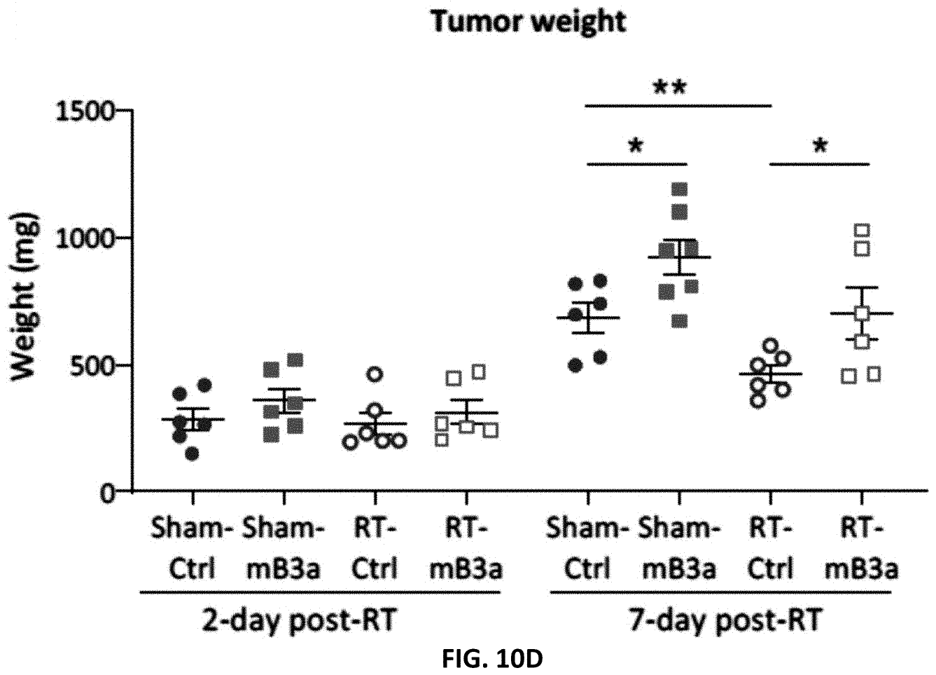

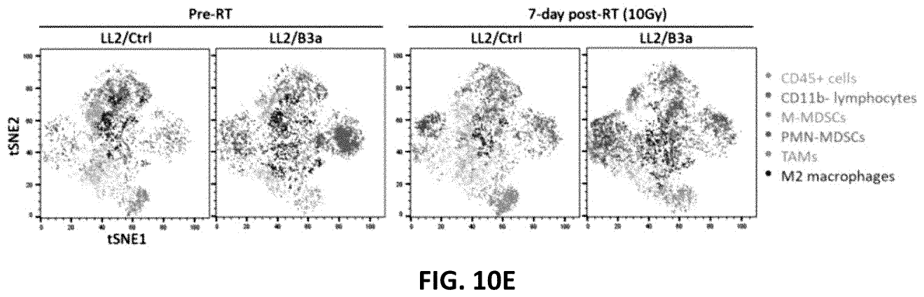

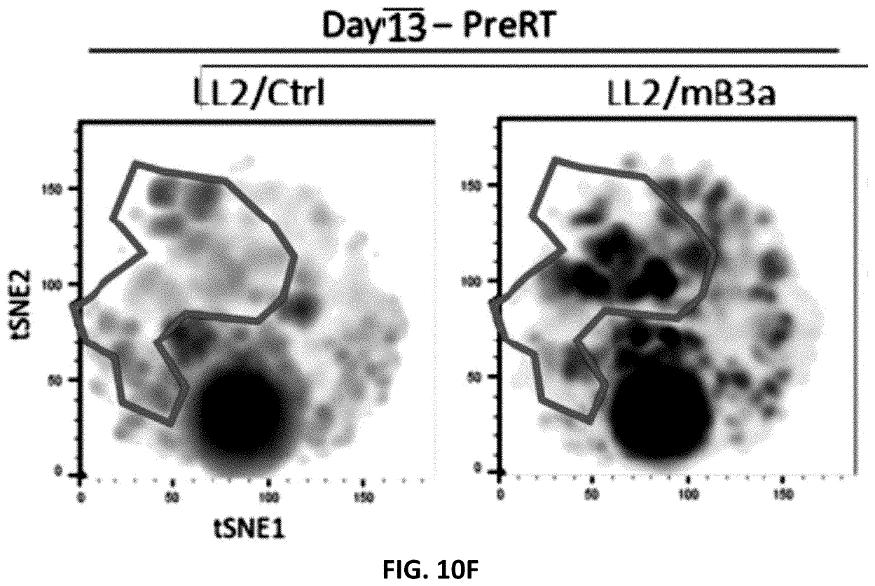

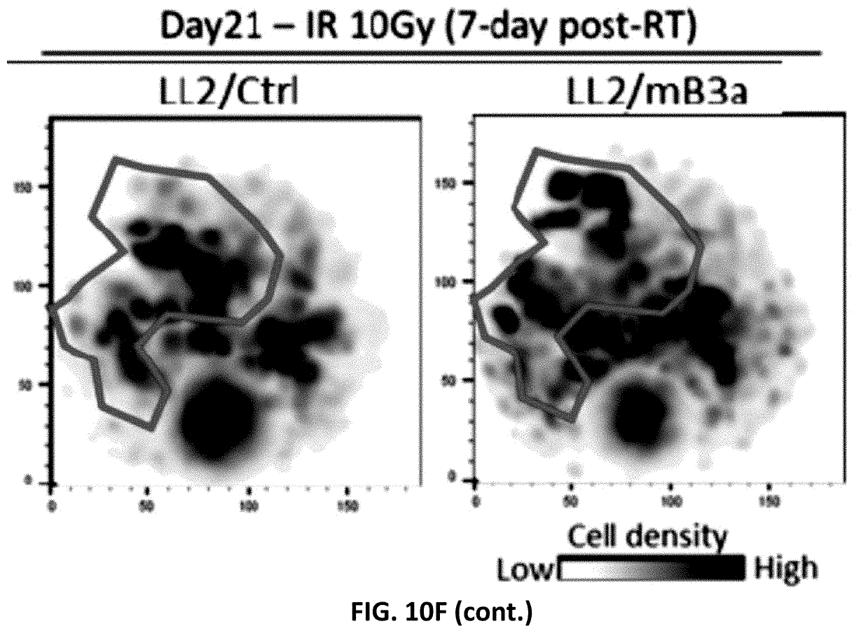

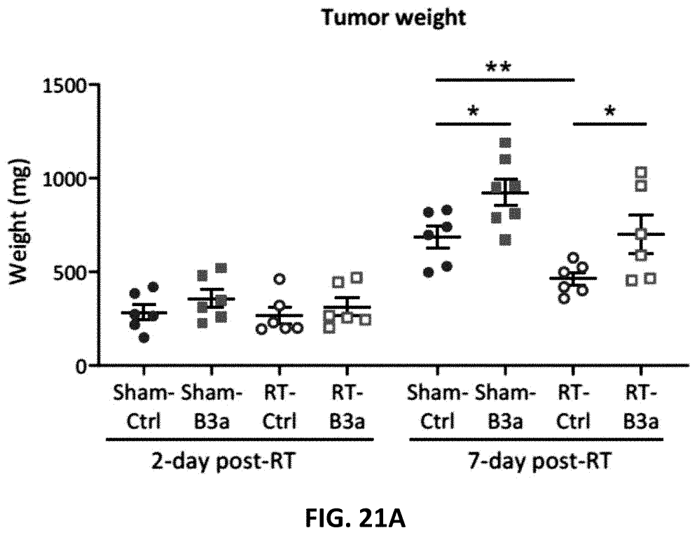

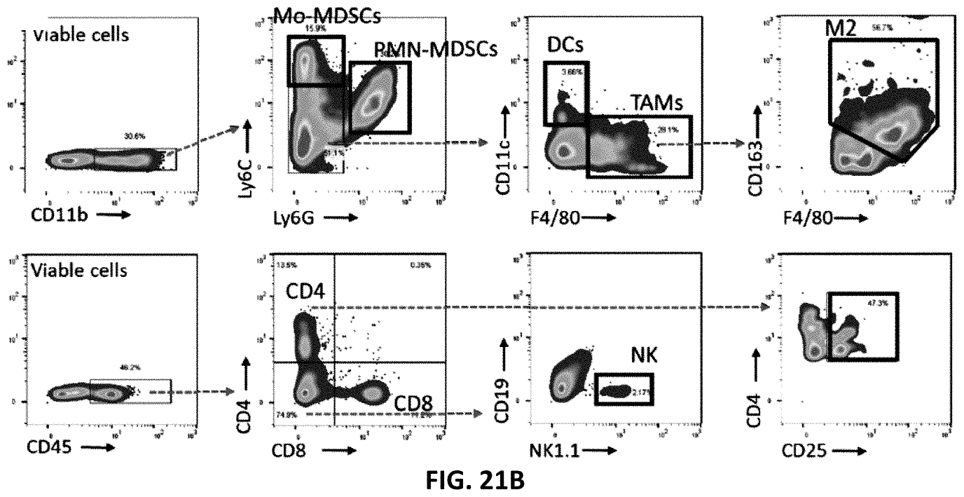

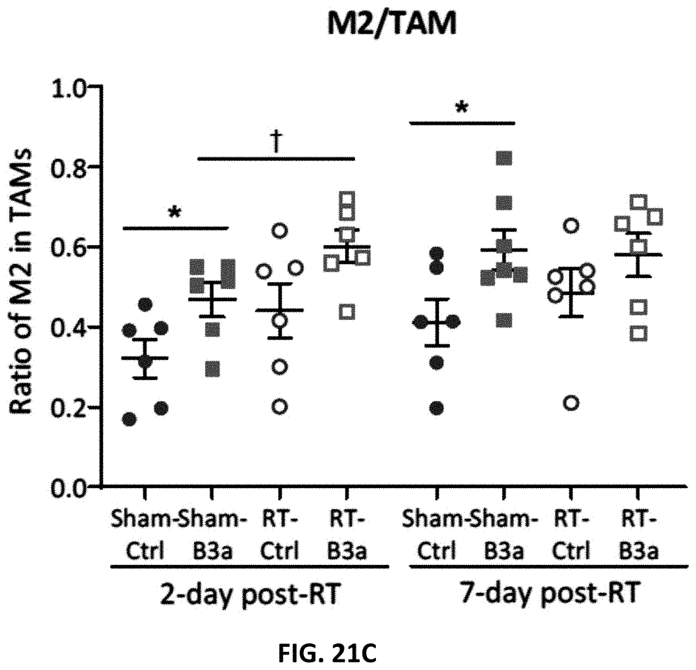

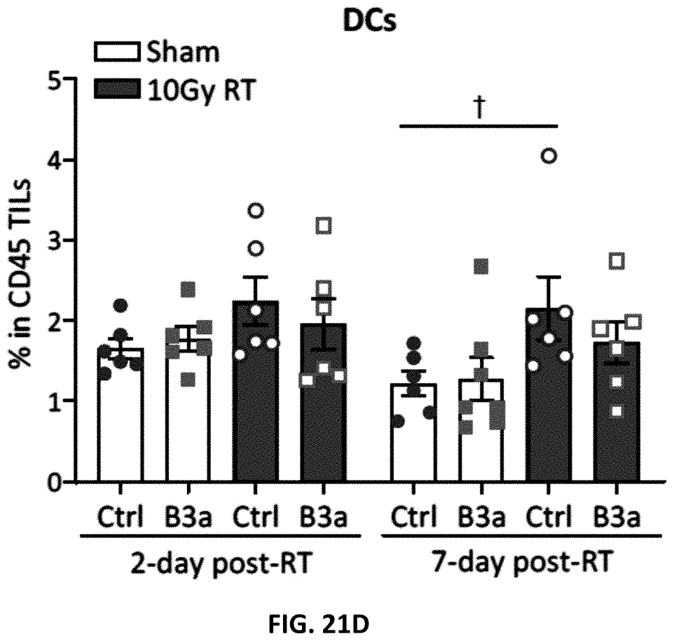

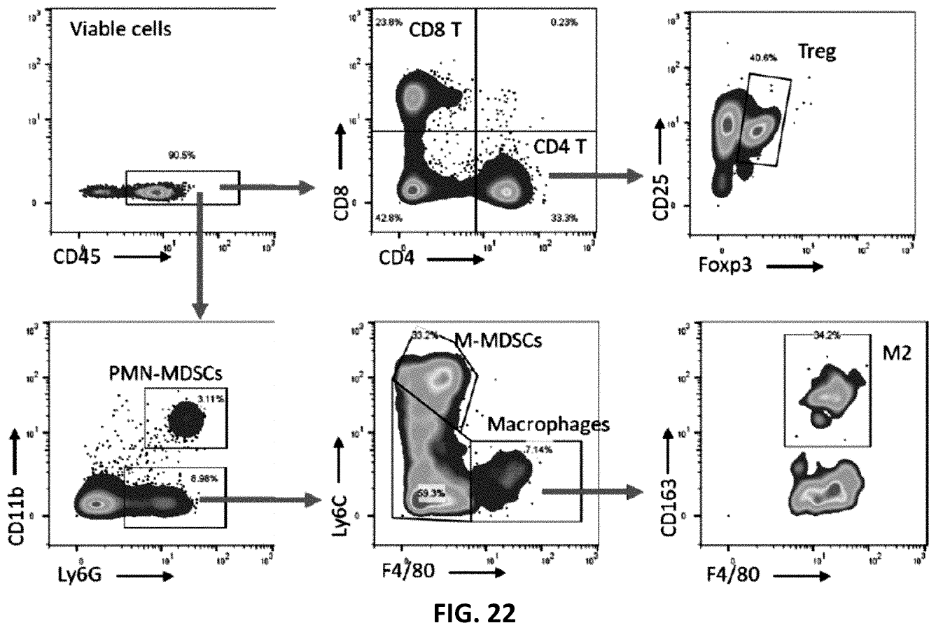

LL2/Ctrl and LL2/B3a cells were injected subcutaneously into C57/BL6 mice, which were then randomized to receive a single dose of 10 Gy or sham RT (14 days post-injection). Immune cell infiltration was analyzed at 2 days post-RT (day 16) and 7 days post-RT (day 21). Tumor growth curves showed that RT-LL2/Ctrl had the smallest tumor volumes and RT-L2/B3a tumor growth curve overlapped with sham-LL2/Ctrl tumors, while sham-LL2/B3a showed the fastest tumor growth (FIGS. 10C and D). This is consistent with a prior study showing that human cervical tumor cell lines expressing SERPINB3 are more radioresistant than control tumors in an athymic nude murine model. Tumor weights showed no statistical differences in all groups at 2-day post-RT while a more substantial increase in sham- and RT-LL2/B3a tumor growth corresponded to increased tumor weight at 7-day post-RT compared to LL2/Ctrl counterpart (FIG. 21A). The visualization of t-distributed stochastic neighbor embedding (viSNE) plots show the unsupervised clustering of CD45+ immune cell subsets based on pre-defined markers in LL2/Ctrl and LL2/B3a tumors (FIG. 21B). The viSNE analysis revealed that LL2/B3a tumors had an overall higher M−/PMN− MDSCs than LL2/Ctrl tumors at both pre-RT and post-RT time points. Both irradiated LL2/Ctrl and LL2/B3a had increased numbers of total CD11b+ myeloid cells, but different subsets were represented (FIG. 10E). Thus, the dynamic change of immune cell subsets in LL2/Ctrl and LL2/B3a tumors was examined at different time points.

### SERPINB3 Tumors are Enriched for Suppressive Myeloid Cells, Further Augmented by Radiation

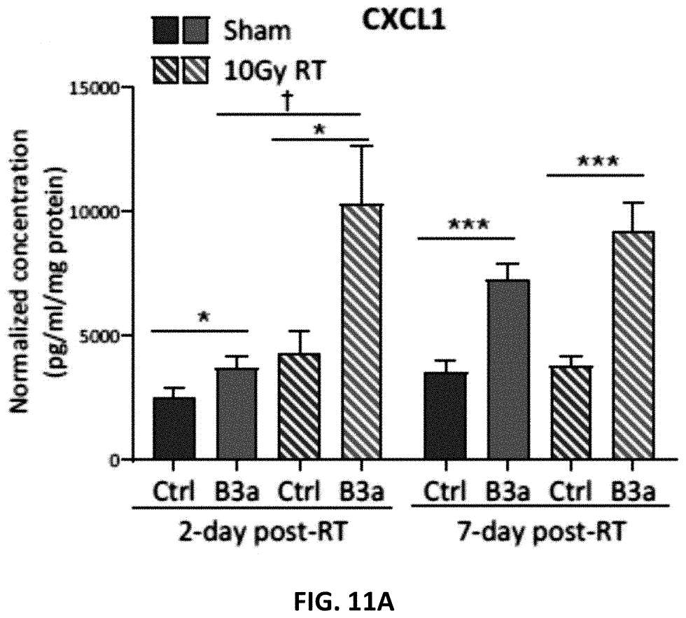

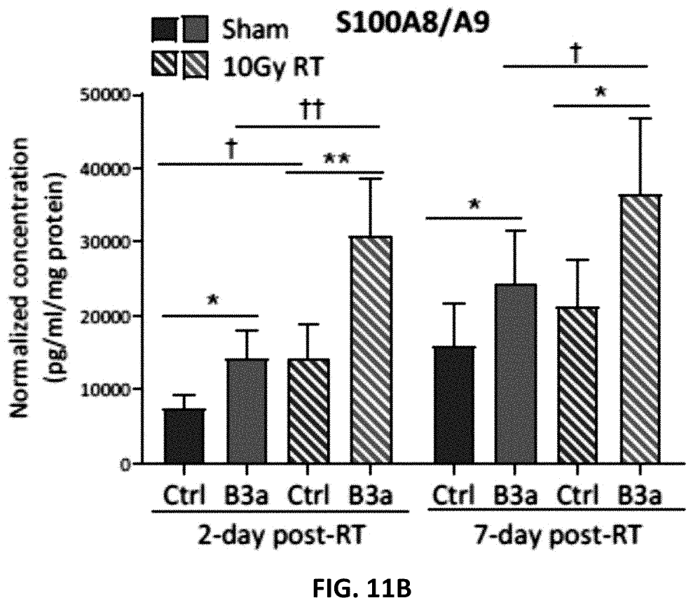

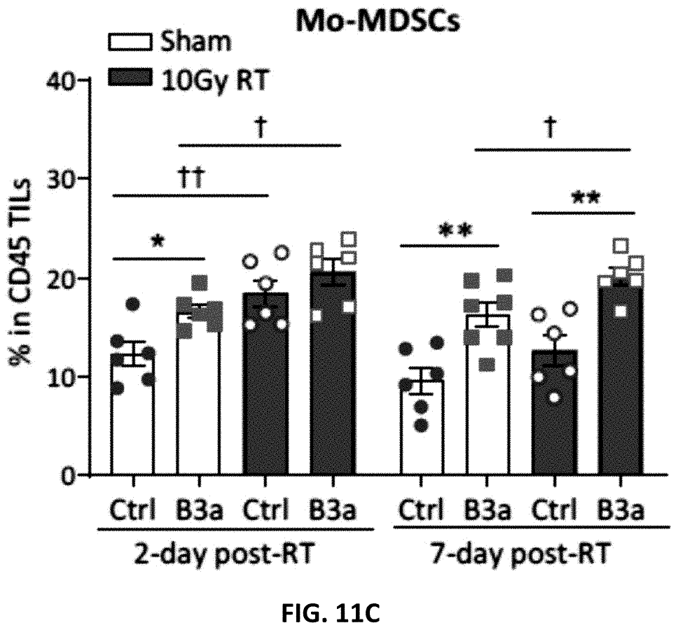

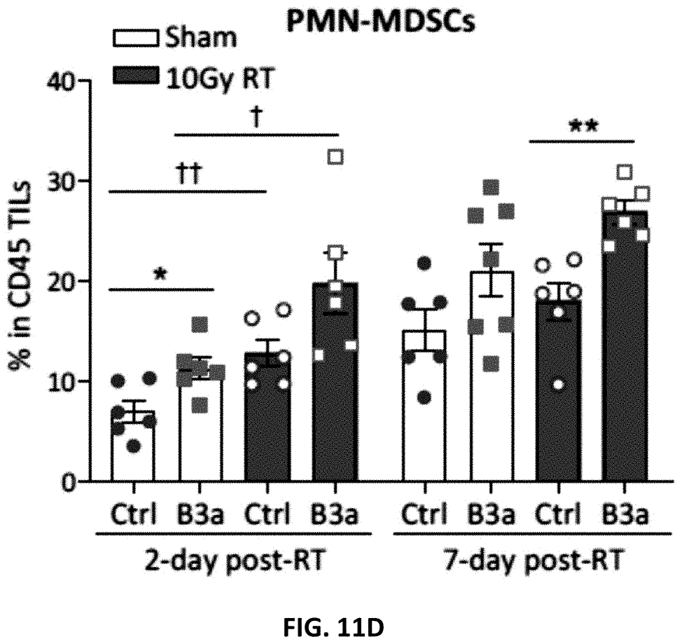

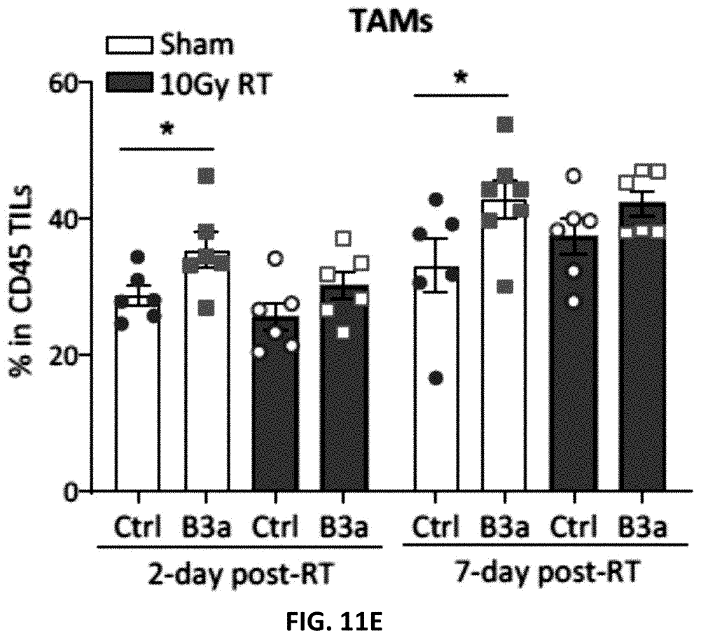

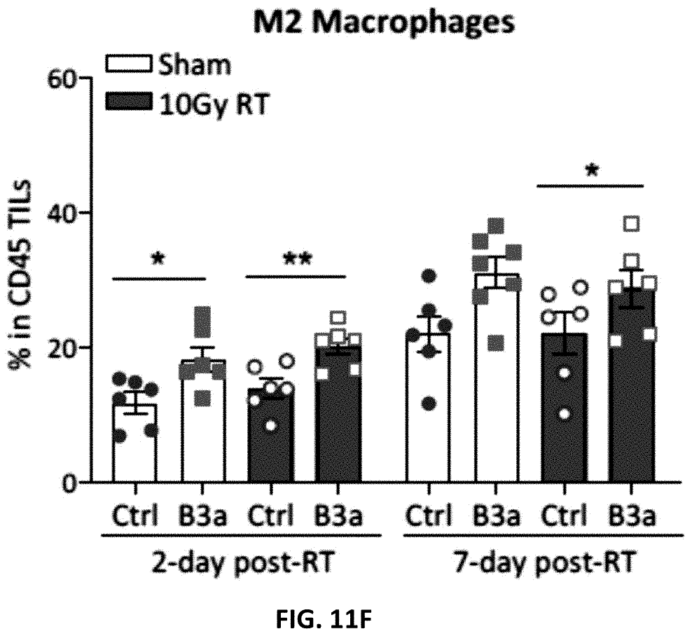

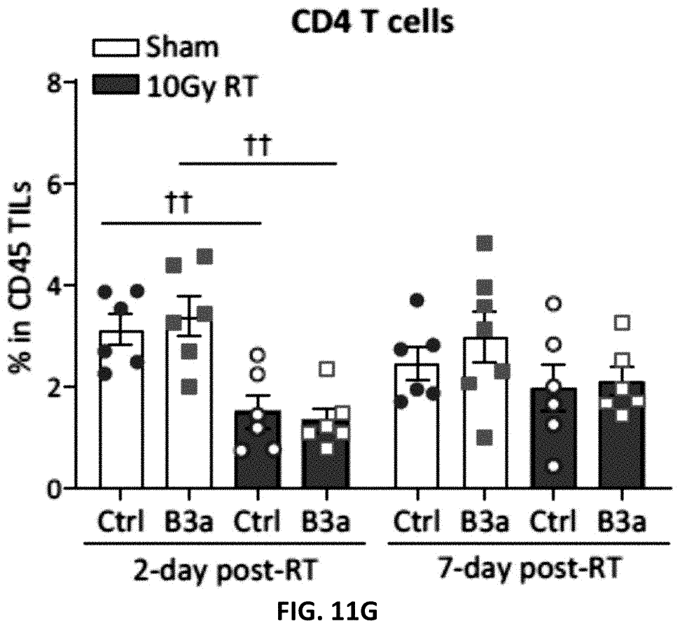

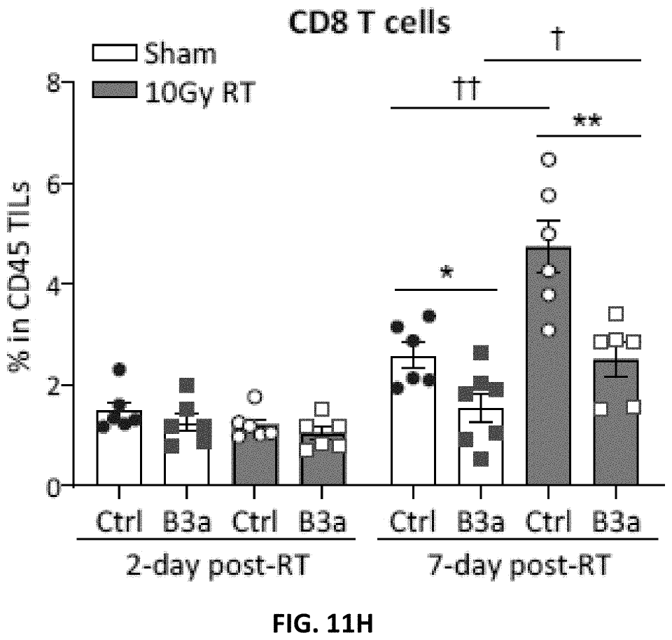

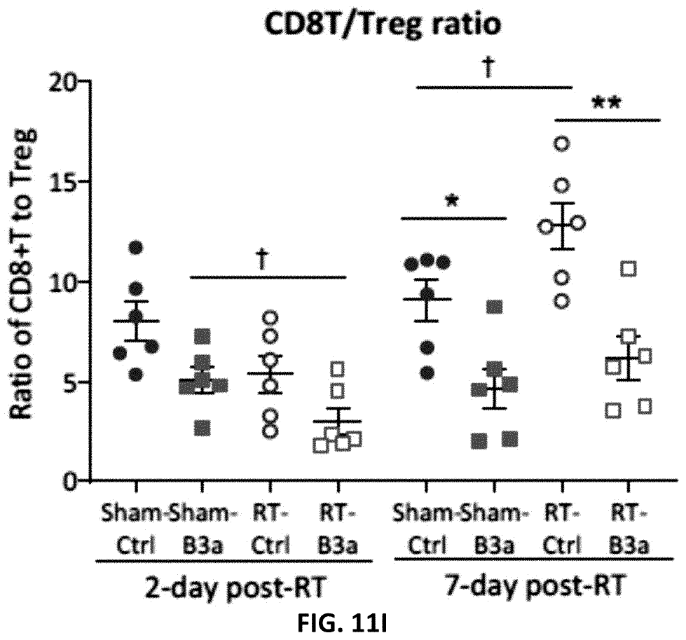

Similar to in vitro findings, LL2/B3a tumors had higher levels of intra-tumoral CXCL1 and S100A8/A9 expression over time, compared to Sham-LL2/Ctrl (FIGS. 11A and B). Radiation promoted further CXCL1 production in RT-LL2/B3a but not in RT-LL2/Ctrl (FIG. 11A). Although radiation treatment induced S100A8/A9 in both RT-LL2/Ctrl and RT-LL2/B3a tumors at 2 days post-RT, the magnitude of chemokine induction was greater in RT-LL2/B3a than RT-LL2/Ctrl, with an average of 2.3-fold and 1.8-fold increase, respectively (FIG. 11B). Higher and more persistent expression of immunosuppressive chemokines in the tumor milieu of irradiated LL2/B3a tumors led to the hypothesize that the increased myeloid compartment summarized by viSNE plots differed specifically in immunosuppressive myeloid cell subtypes. Indeed, Sham-treated LL2/B3a tumors showed consistently higher infiltration of M-MDSCs and PMN-MDSCs compared to LL2/Ctrl while radiation treatment induced an early increase of infiltrating M- and PMN-MDSCs at 2-day post-RT in both groups; however, MDSCs in irradiated tumors remained elevated compared to sham-treated tumors at 7-days post-RT only in RT-LL2/B3a tumors (FIGS. 11C and D). The number of infiltrating TAMs and M2 macrophages was higher in sham LL2/B3a versus LL2/Ctrl, with a gradual increase in both groups as the tumors grew, but no statistical change with irradiation in either genetic background (FIGS. 11E and F). The ratio of M2 macrophages to total TAMs, however, was significantly higher in sham-treated LL2/B3a compared to LL2/Ctrl, with a further increase induced by radiation only in the LL2/B3a tumors (FIG. 21C).

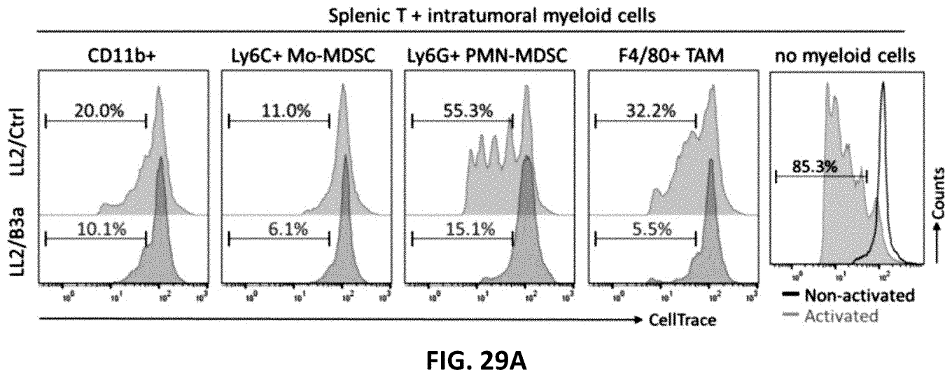

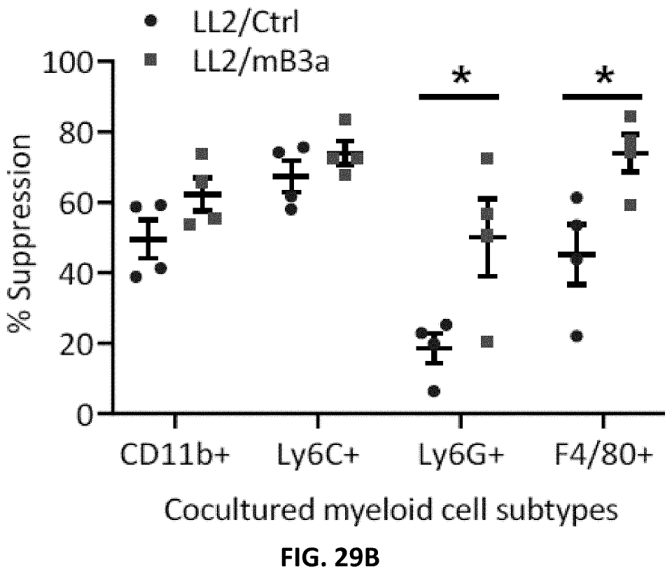

To assess the immunosuppressive activity of myeloid cells from LL2/Ctrl and LL2/B3a tumors, intratumoral CD11b+ myeloid cells, Ly6C+ Mo-MDSCs, Ly6G+ PMN-MDSCs, and F4/80+ TAMs were isolated and co-cultured with splenic T cells derived from non-tumor bearing mice. Intratumoral Ly6C+ Mo-MDSCs from both LL2/Ctrl and LL2/B3a tumors demonstrated strong inhibition towards T cell proliferation. Notably, Ly6G+ PMN-MDSCs and F4/80+ TAMs from LL2/B3a tumors had more significant inhibitory effects than those from LL2/Ctrl (FIGS. 29A and B). In LL2/B3a tumors, the increased release of CXCL1 and S100A8/A9 upon radiation corresponded to increased suppressive myeloid cell infiltrates, contributing to an exacerbated RT-induced immunosuppressive response.

Cytotoxic T Cells from SERPINB3tumors Display Impaired Proliferation and Exhausted Phenotypes

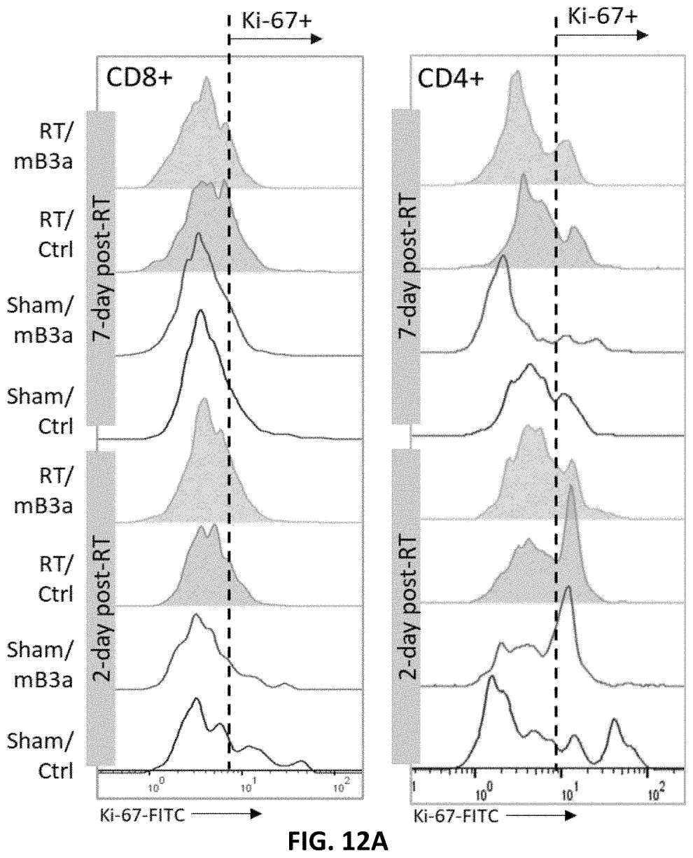

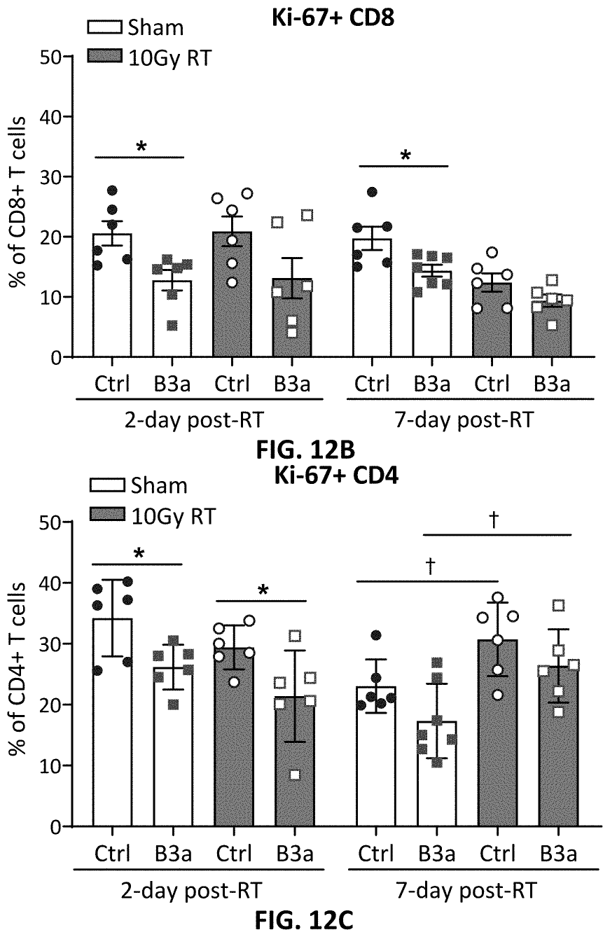

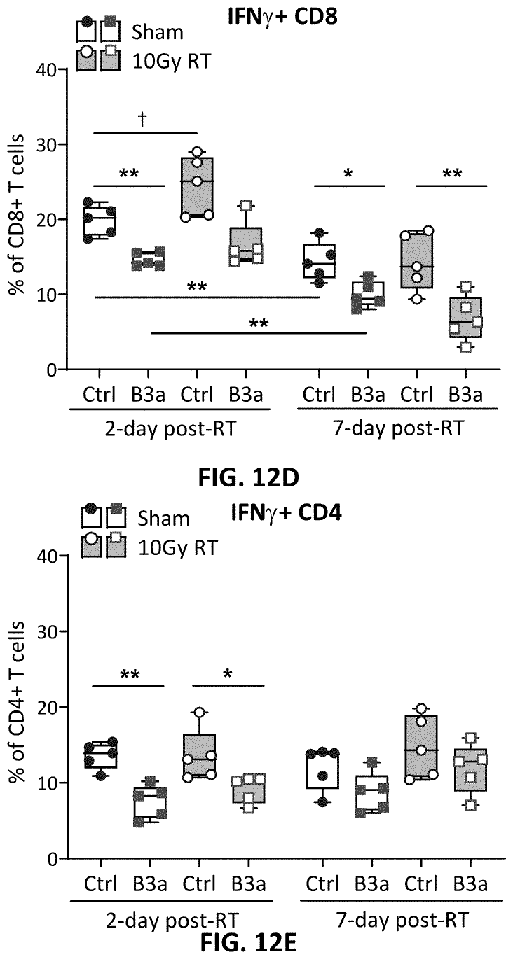

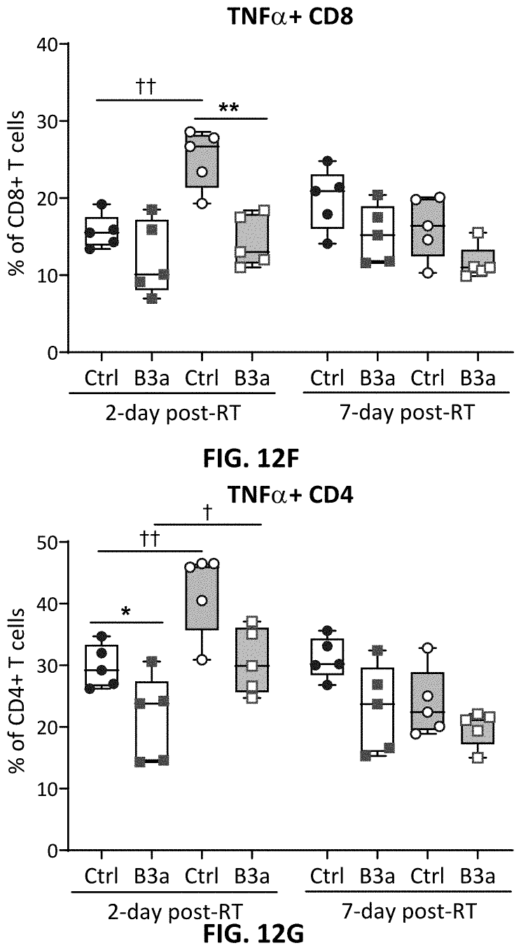

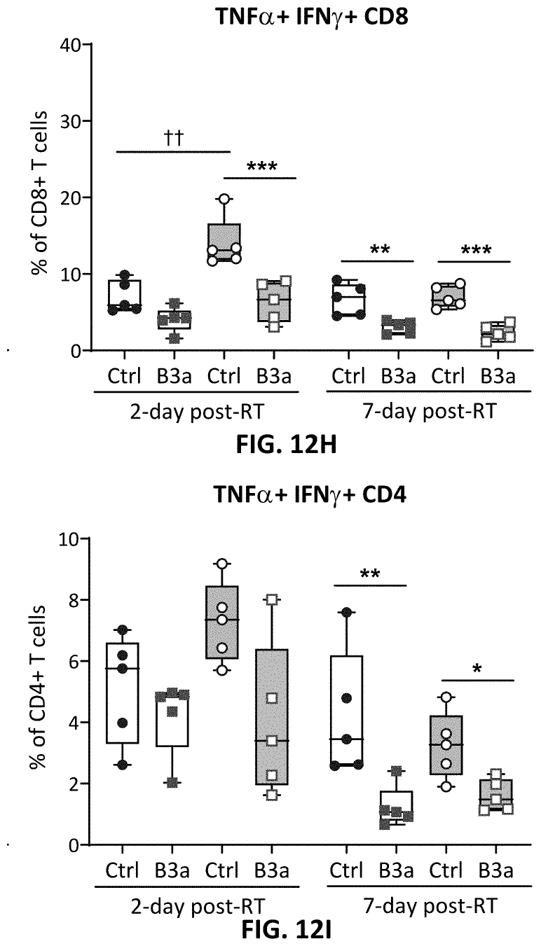

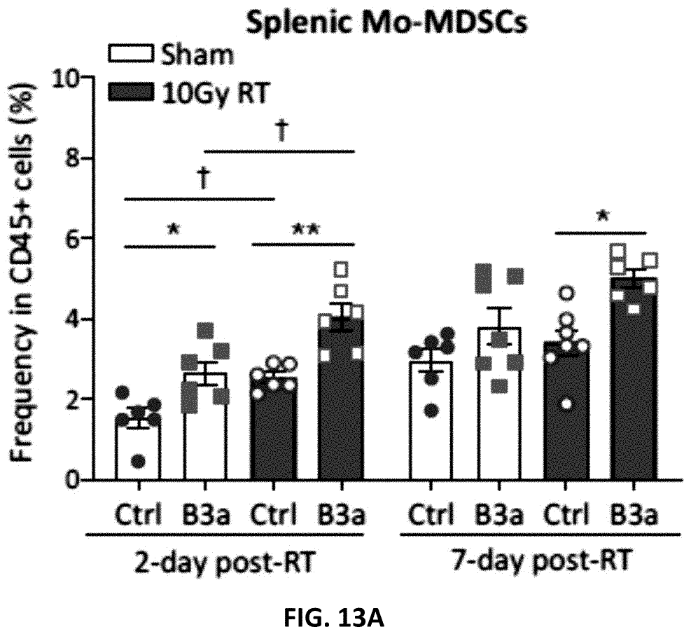

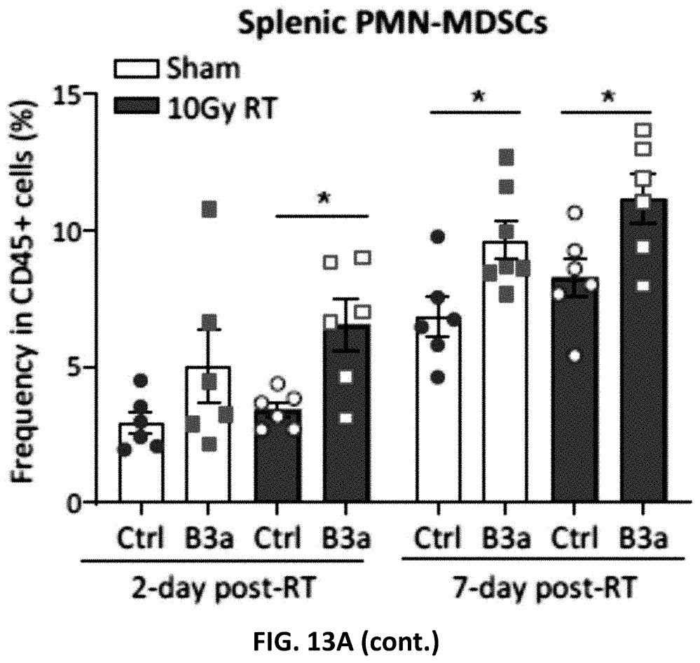

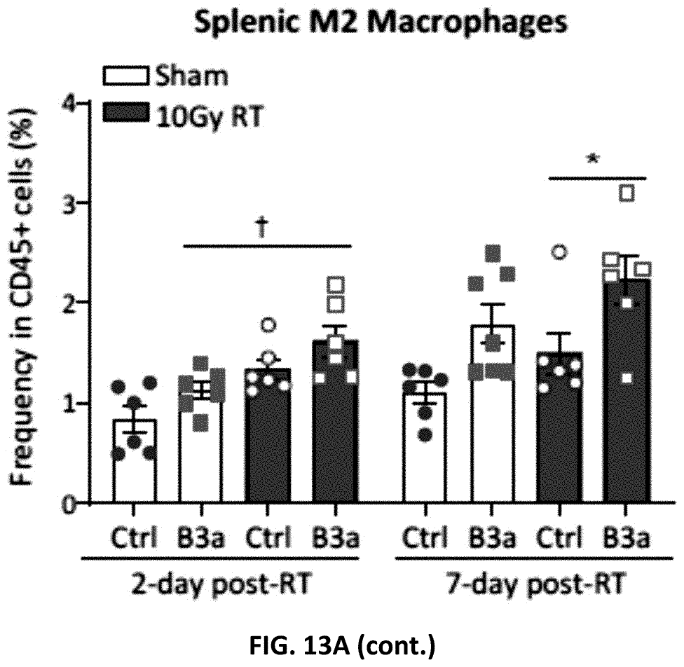

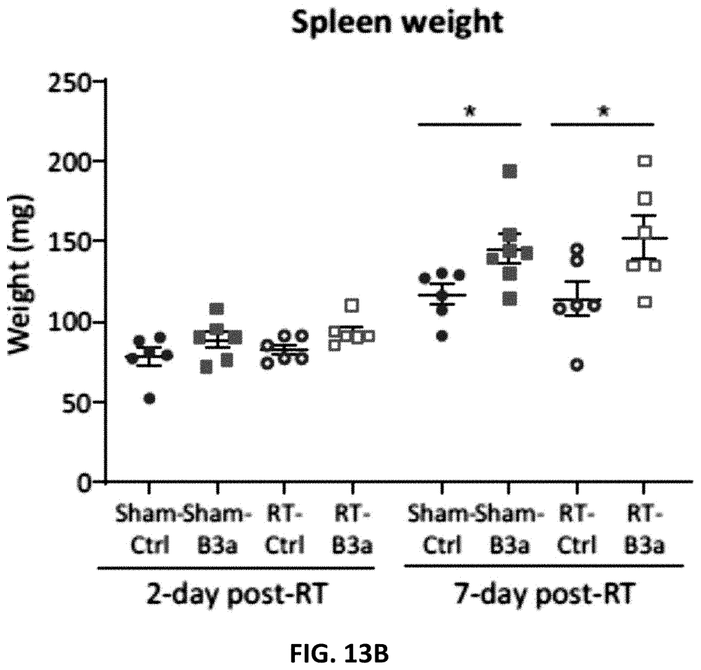

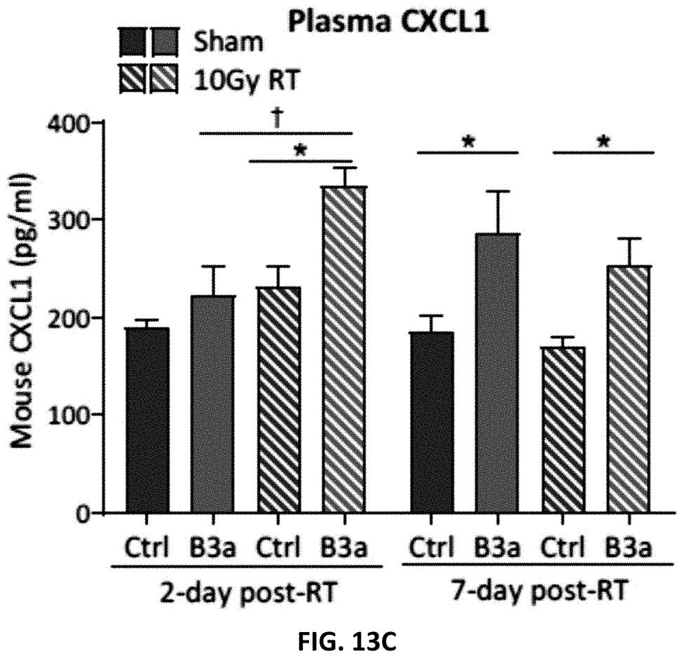

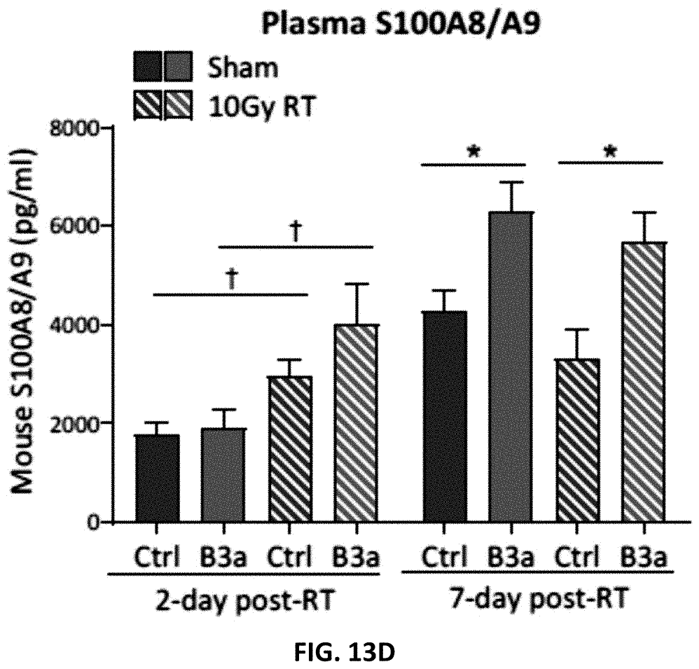

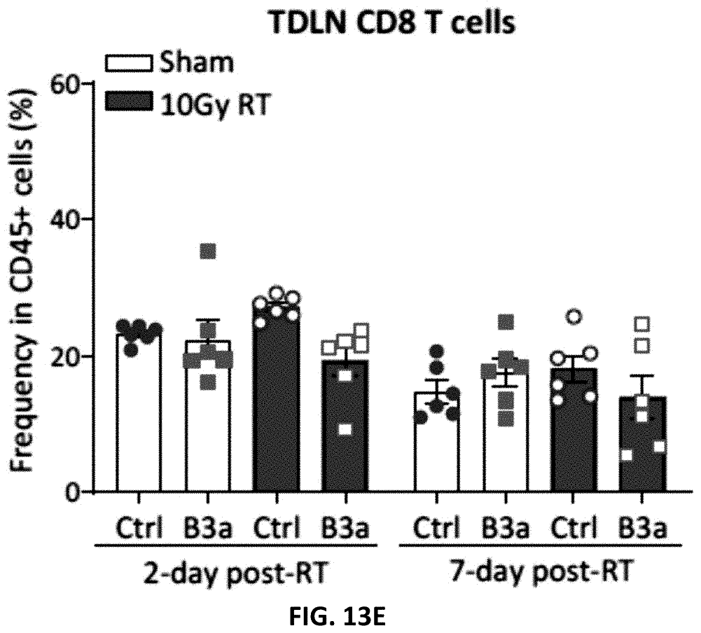

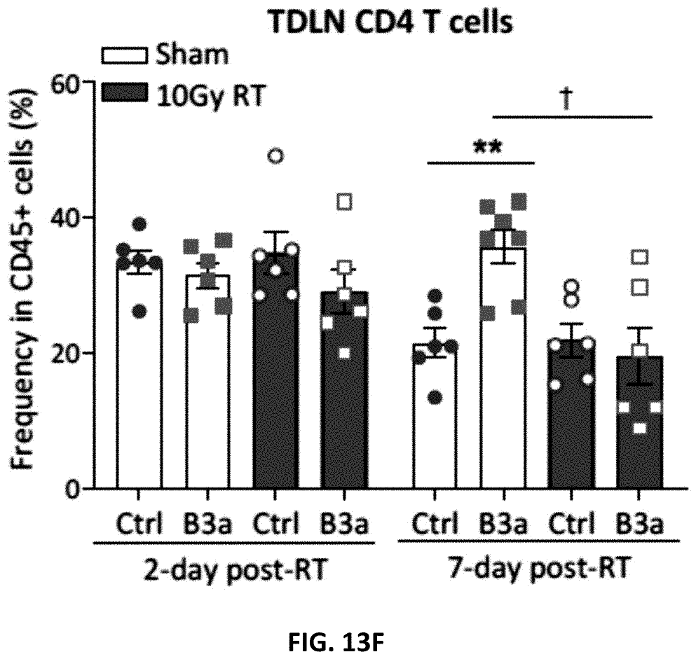

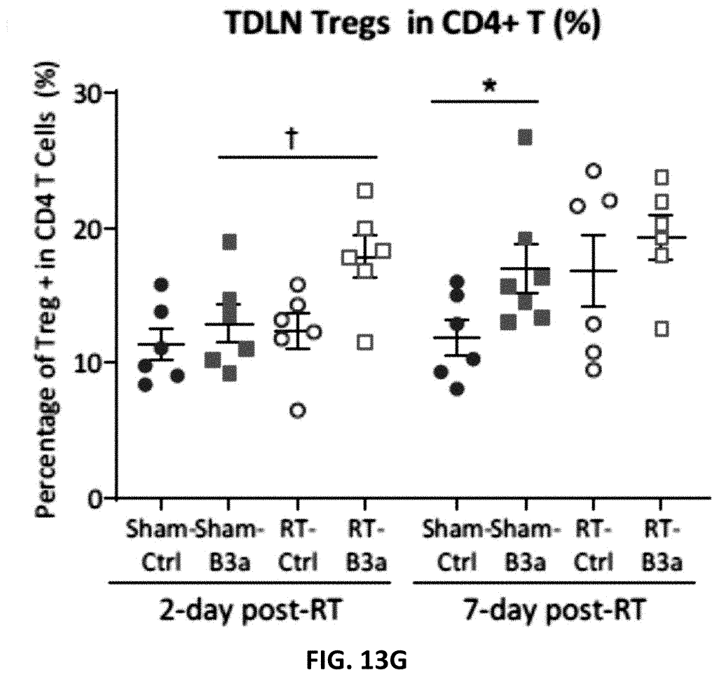

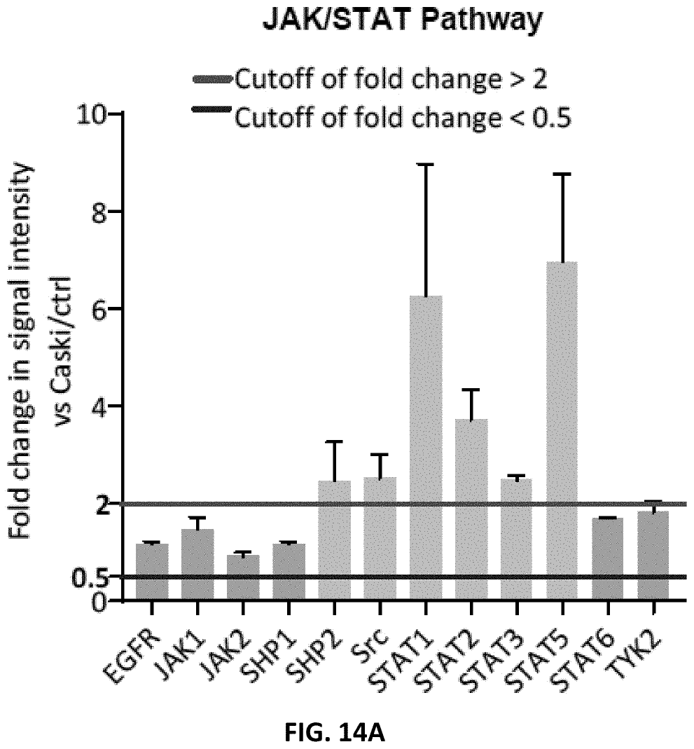

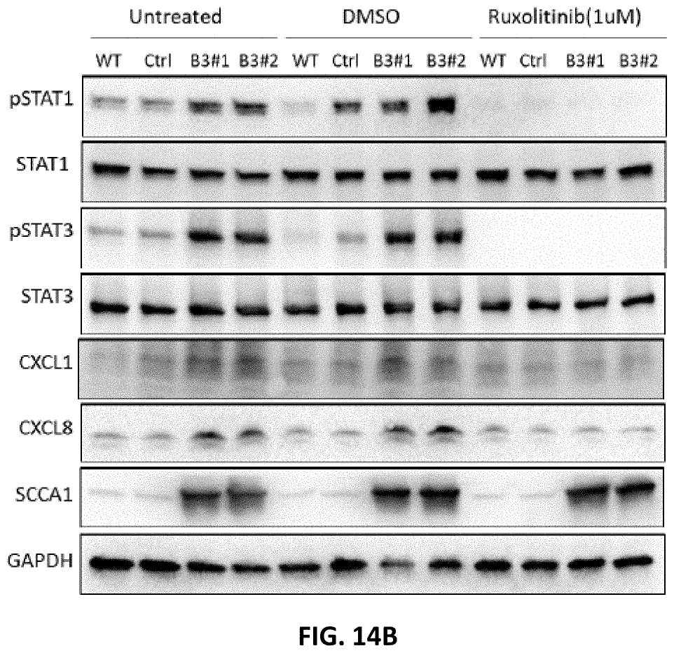

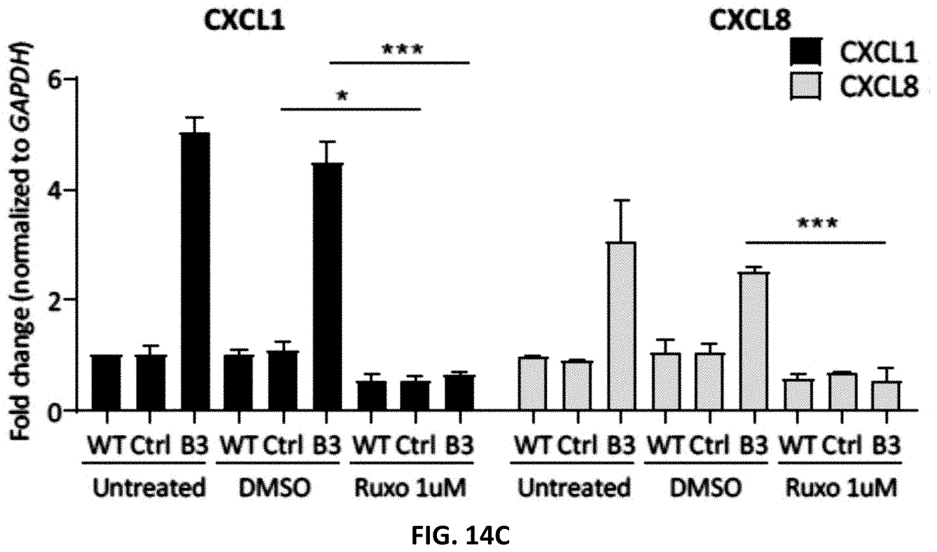

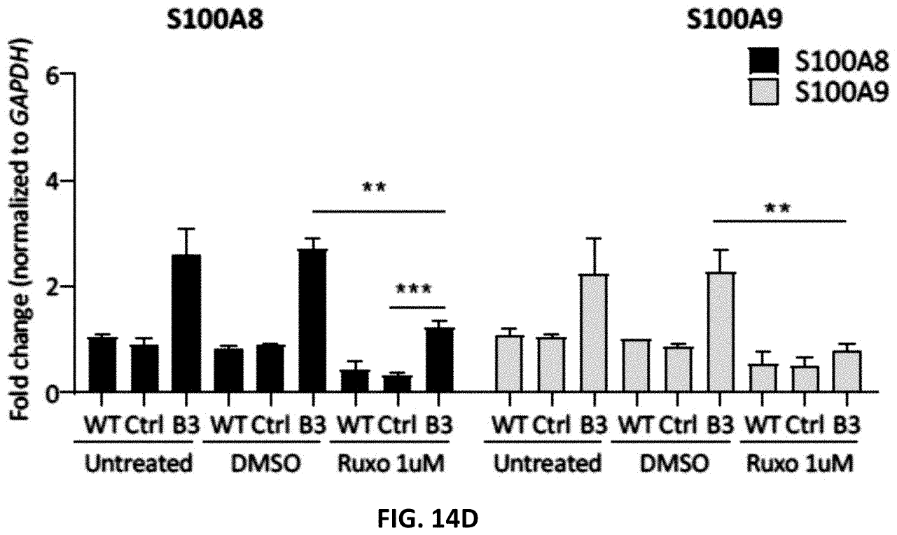

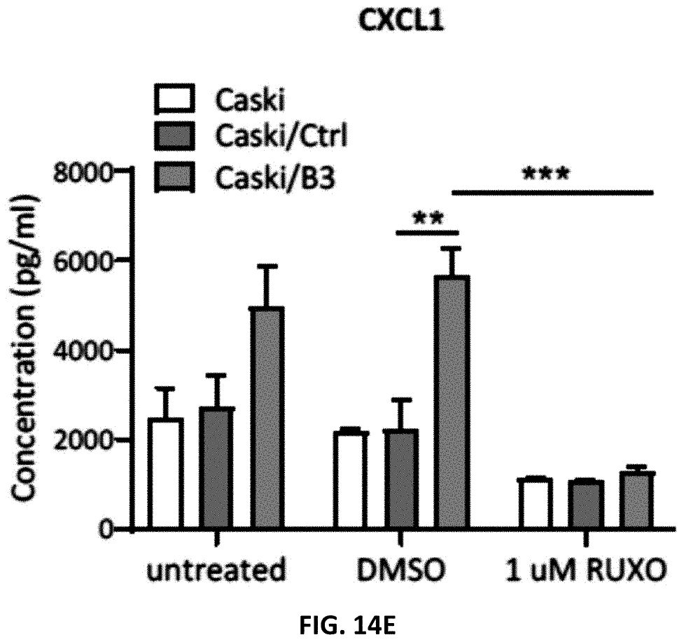

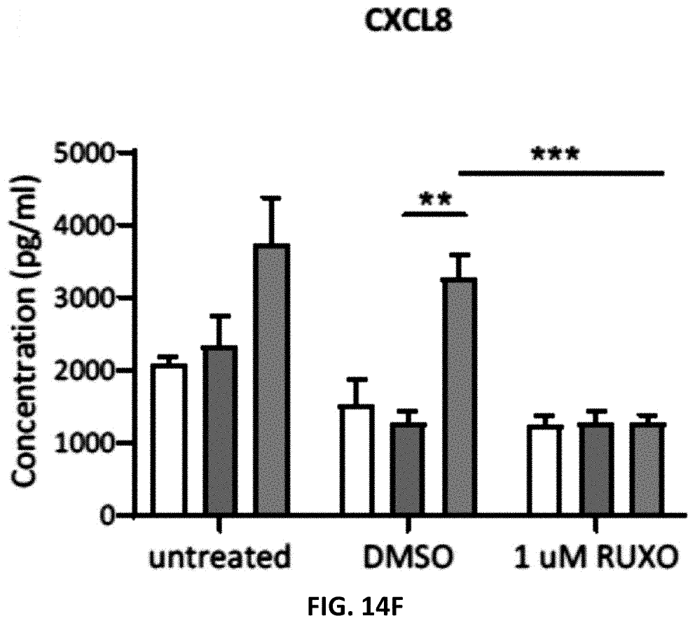

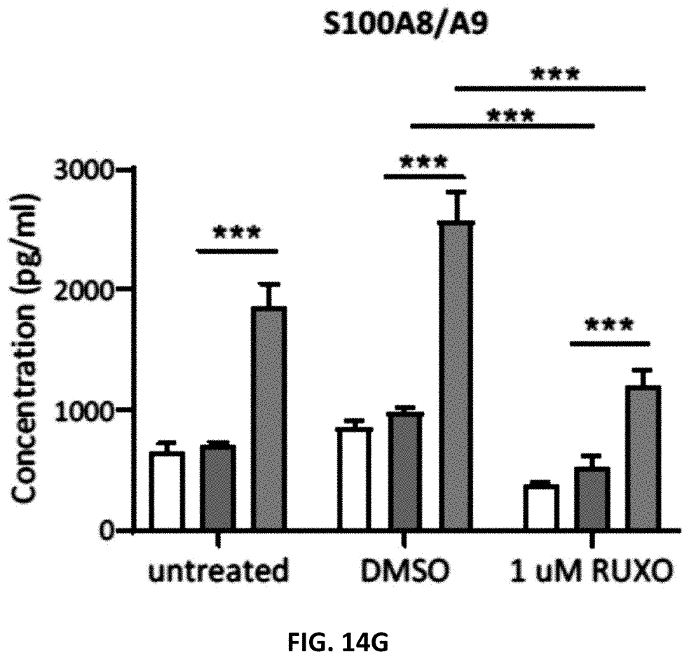

With evidence of immunosuppressive TME, T cell recruitment and function are likely to be compromised in LL2/B3a tumors. CD8+ TILs showed significantly lower in Sham-/RT-LL2/B3a vs in LL2/Ctrl at 7 days post-RT. In RT-LL2/Ctrl tumors, CD8+ TILs doubled compared to sham-treated tumors, and while statistically increased, the magnitude of increase was less in RT-LL2/B3a (FIG. 11H). No difference was seen in CD4+ TILs between LL2/Ctrl and LL2/B3a tumors and a significant decrease at 2-day post-RT in both groups was associated with radiation effect (FIG. 11G), consistent with radiosensitivity of in-field lymphocytes. The ratio of CD8+ T to Treg (CD4+ CD25+ FoxP3+) was significantly decreased in RT-LL2/B3a compared to Sham-LL2/B3a, indicating an increase of Treg cells in LL2/B3a tumors shortly after radiation. In contrast, increased CD8+ TILs in RT-LL2/Ctrl at 7-day post-RT correlated with higher CD8+ T/Treg ratio compared to sham-LL2/Ctrl tumors (FIG. 11I). The analysis of proliferation marker Ki-67 expression in cytotoxicCD8+ T cells showed that percent of Ki-67+CD8+ TILs were lower in both Sham- and RT-treated LL2/B3a compared to LL2/Ctrl tumors at 2-day post-RT, despite comparable numbers of infiltrating CD8+ TILs. Tumor-directed radiation promoted CD8+ T cell infiltration but not proliferation at 7-day post-RT as most CD8+ T cells showed low Ki-67 expression in both LL2/Ctrl and LL2/B3a tumors (FIGS. 12A and B).

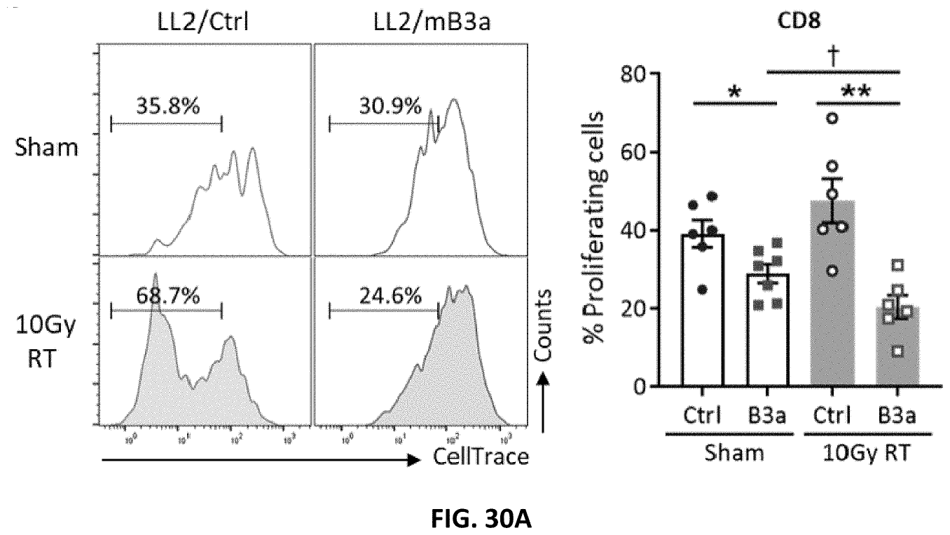

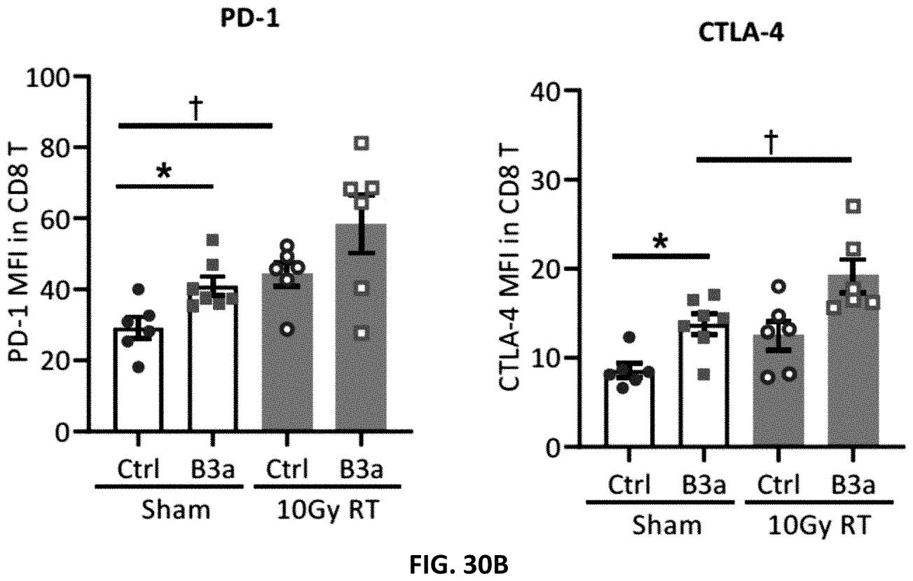

Cytotoxic CD8+ T cells were further evaluated for the production of interferon gamma (IFNγ) and tumor necrosis factor alpha (TNFα) following ex vivo stimulation with PMA/ionomycin. An average of 20% of sham-treated CD8+ TILs from LL2/Ctrl and 15% from LL2/B3a tumors showed IFNγ production while the frequencies of IFNγ-producing CD8+ TILs were reduced with tumor growth in both groups (FIG. 12D). CD8+ TILs taken from RT-LL2/Ctrl tumors at 2-day post-RT showed significant enhancement of both IFNγ and TNFα production following stimulation, whereas radiation-boosted IFNγ and TNFα production was not observed in CD8+ TILs from RT-LL2/B3a tumors (FIGS. 12D and F). T cell receptor (TCR)-mediated activation was examined by the proliferation of CellTrace-labeled CD8+ TILs stimulated with anti-CD3/28 antibody. CD8+ TILs from sham and RT LL2/Ctrl tumors demonstrated stronger proliferative capacity than those derived from LL2/B3a tumors. Radiation did not have significant effects on the TCR-mediated proliferation of CD8+ TILs from LL2/Ctrl while decreased proliferation was observed in CD8+ TILs from LL2/B3a tumors (FIG. 30A). The impaired proliferation and decreased IFNγ and TNFα production might suggest an exhausted phenotype. Indeed, increased expression of PD-1 and CTLA-4 was observed in LL2/B3a-derived CD8+ TILs compared with LL2/Ctrl-derived CD8+ TILs. Radiation further promoted CTLA-4 expression in LL2/B3a-derived CD8+ TILs, indicating increased T cell exhaustion in LL2/B3a tumors (FIG. 30B).

### Depleting CD11b+ Myeloid Cells in SERPINB3 Tumors Improves T Cell Activity

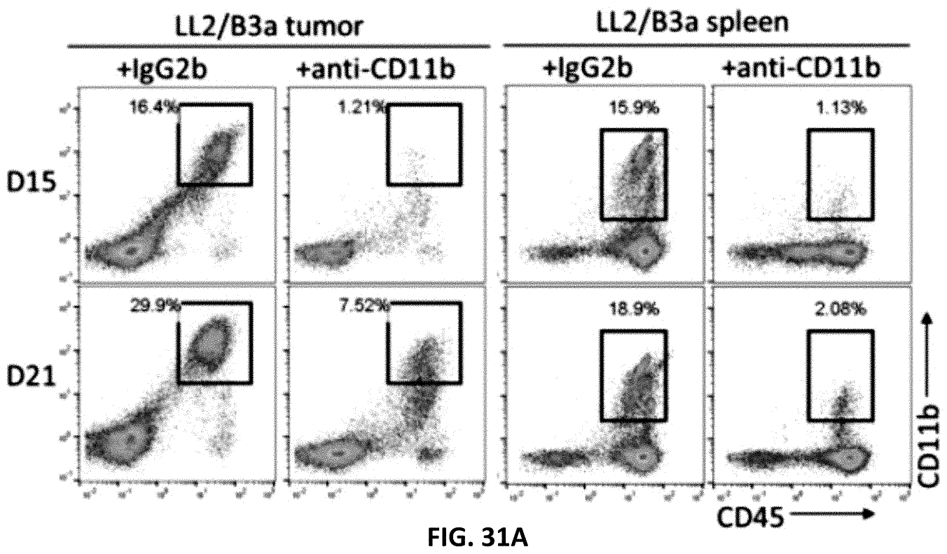

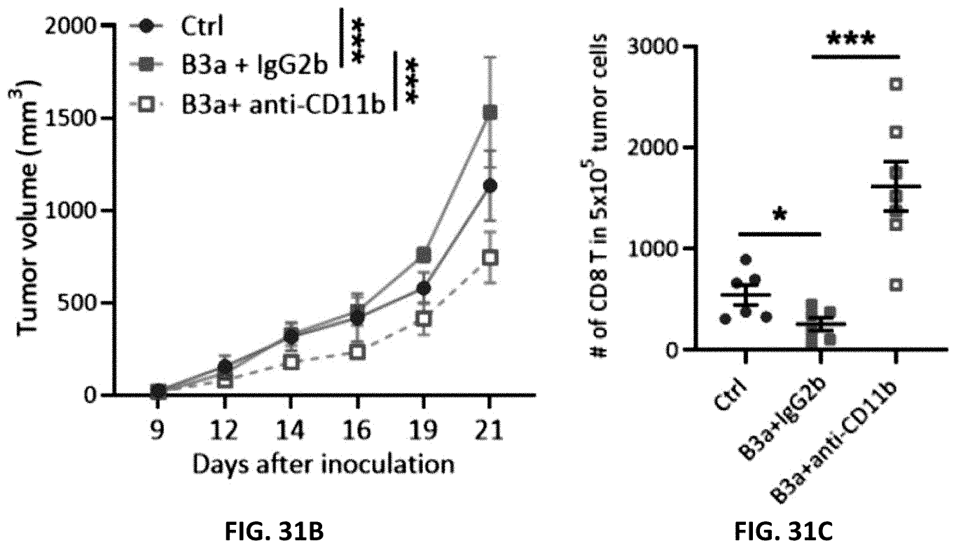

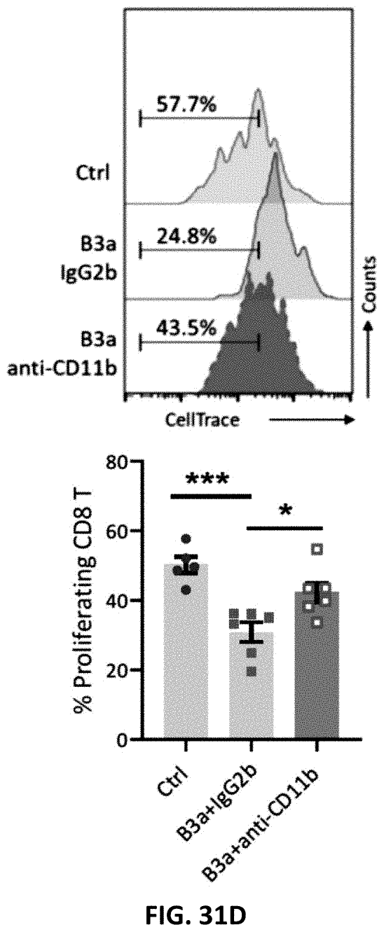

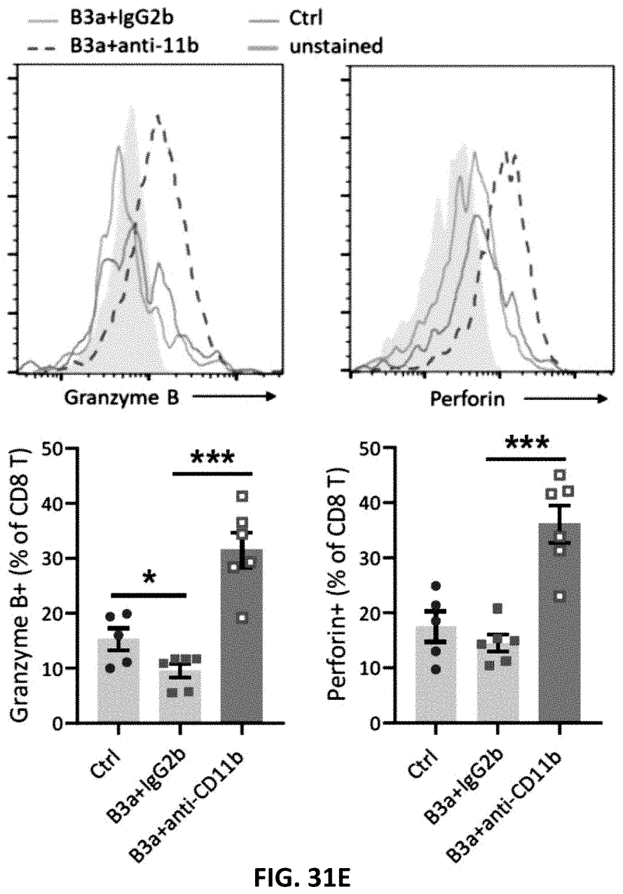

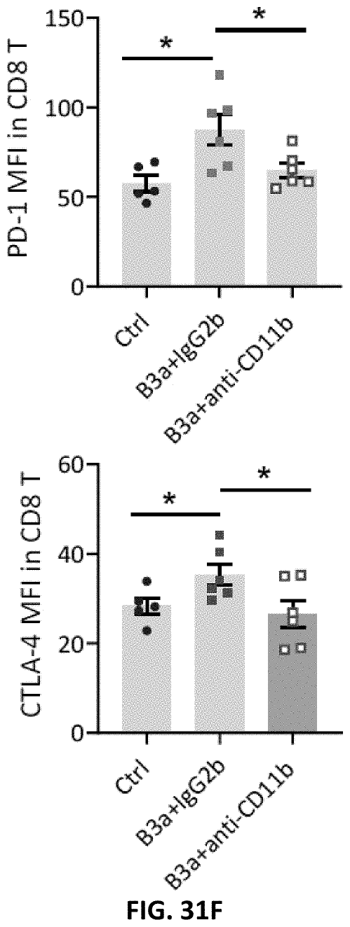

To determine whether impaired T cell activity in LL2/B3a tumors was associated with high infiltration of immunosuppressive myeloid cells, we treated LL2/B3a-tumor bearing mice with CD11b neutralizing antibody to deplete myeloid cells or IgG2b isotype control starting on day 9 post-tumor inoculation. Splenic and intratumoral depletion of CD11b+ cells was examined on day 15 and 21, where efficient depletion was observed in spleen on both days but slightly recovered in tumors on day 21 (FIG. 31A). The growth of LL2/B3a tumors significantly reduced by anti-CD11b antibody treatment compared to LL2/B3a treated with IgG2b control or LL2/Ctrl tumors (FIG. 31B). A decreased total number of CD8+ T cells in LL2/B3a compared to LL2/Ctrl tumors was reversed by the depletion of CD11b+ cells (FIG. 31C). This also relieved the suppression of CD8+ T cells to enhance their activity in LL2/B3a tumors, where less responsive CD8+ T cells to CD3/28− induced activation was increased in anti-CD11b-treated LL2/B3a tumors (FIG. 31D). The expression of cytotoxic granules, perforin+ and granzyme B+ CD8+ T cells were significantly increased in anti-CD11b-treated LL2/B3a tumors compared with IgG2b-treated LL2/B3a and LL2/Ctrl tumors (FIG. 31E). The improved T cell activity in anti-CD11b-treated LL2/B3a tumors was accompanied by reduced PD-1 and CTLA-4 expression, which were highly expressed in IgG2b-treated LL2/B3a tumors (FIG. 31F).

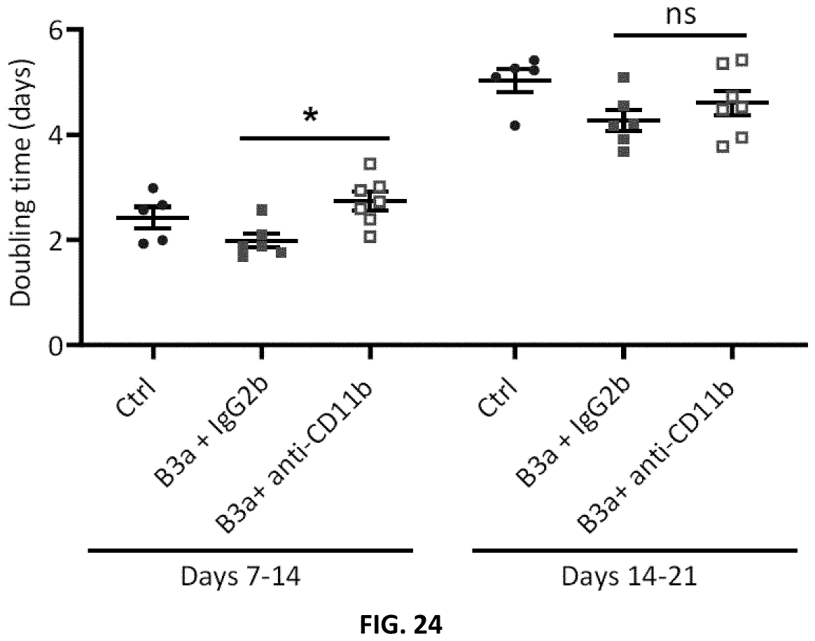

High numbers of myeloid cells in LL2/B3a tumors can be a therapeutic target to restore T cell anti-tumor response; however, clinical trials targeting myeloid cell integrin such as CD11b/CD18 have failed to yield therapeutic benefits, due to the limitation in tolerable doses in human. It was also found that even though tumor size was smaller during CD11b antibody treatment, tumor doubling time remained the same from day 14-21, suggesting that once tumors were established, the growth of tumor cells was not inhibited by CD11b+ cell depletion (FIG. 24). This may be in part due to a multifaceted role of CXCL1 and S100A8/A9 secreted by LL2/B3a tumors in promoting tumor cell proliferation and survival. Therefore, targeting SERIPNB3 may be an alternative approach to reduce tumor growth and provide therapeutic potential.

Targeting SERPINB3 Sensitizes Tumors to Radiation-Induced Remission and T Cell Responses

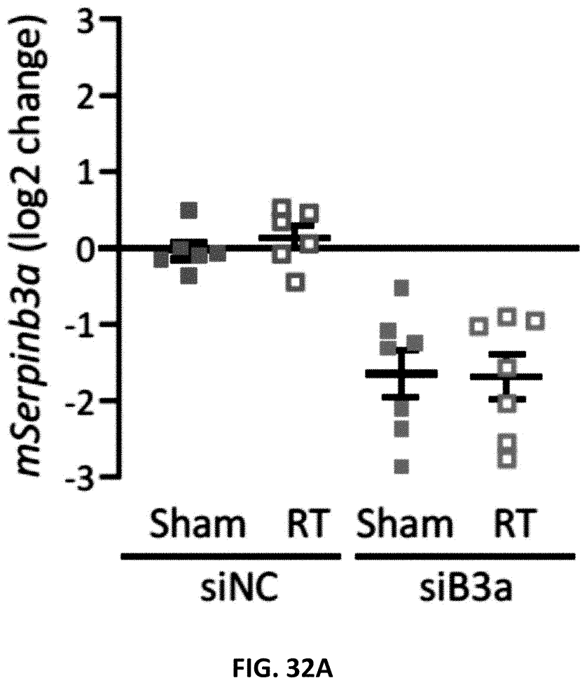

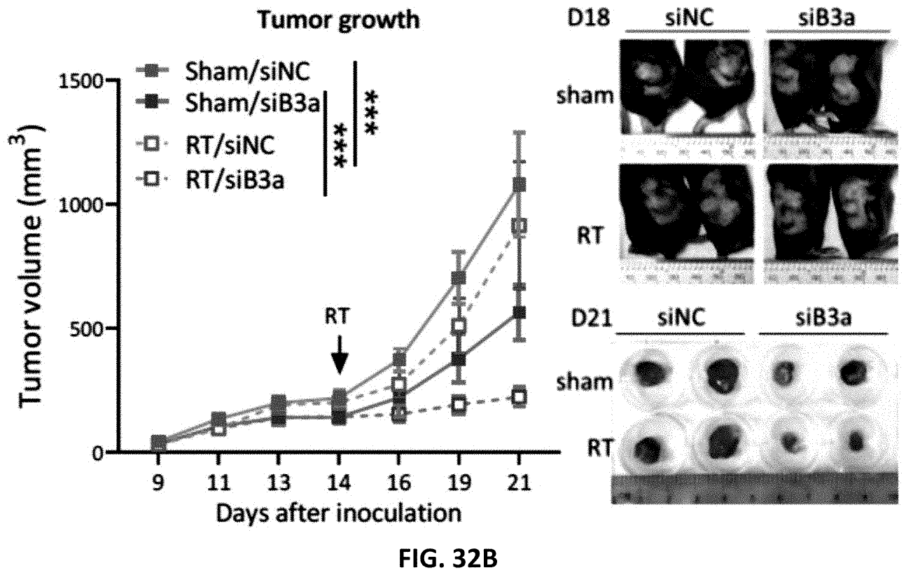

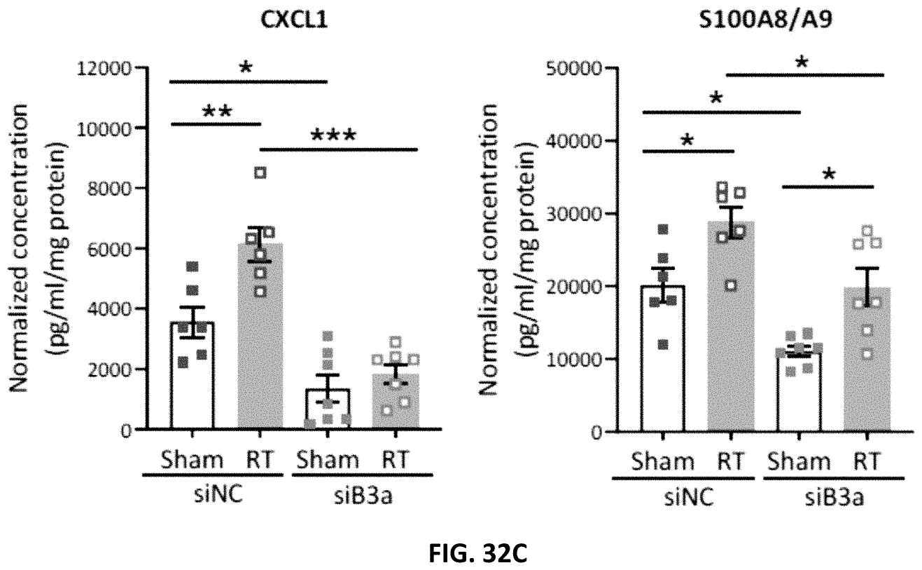

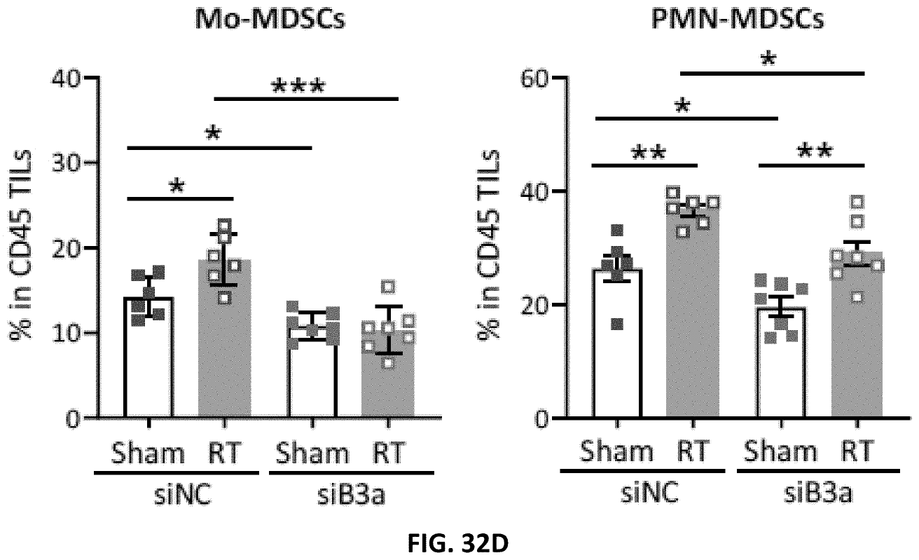

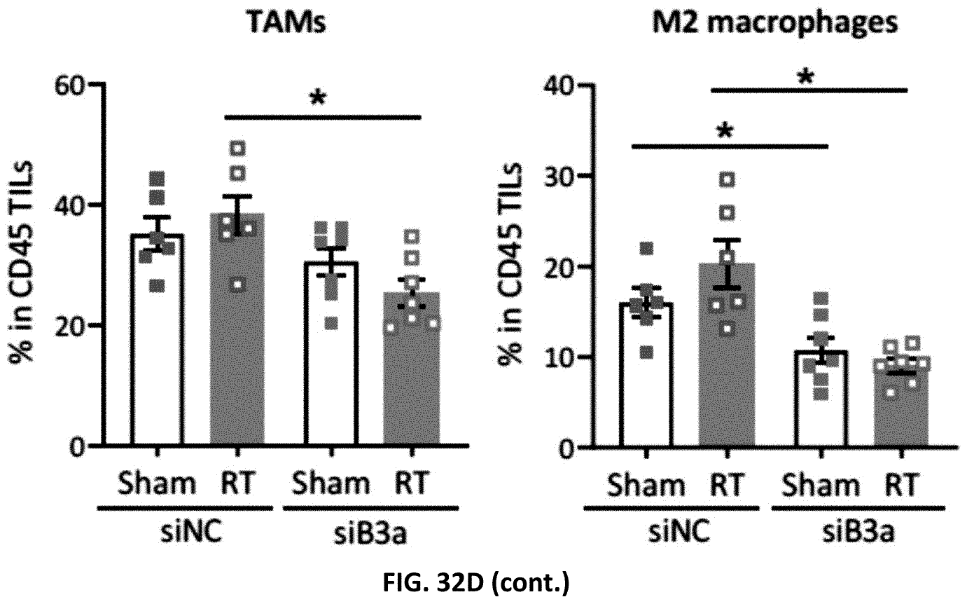

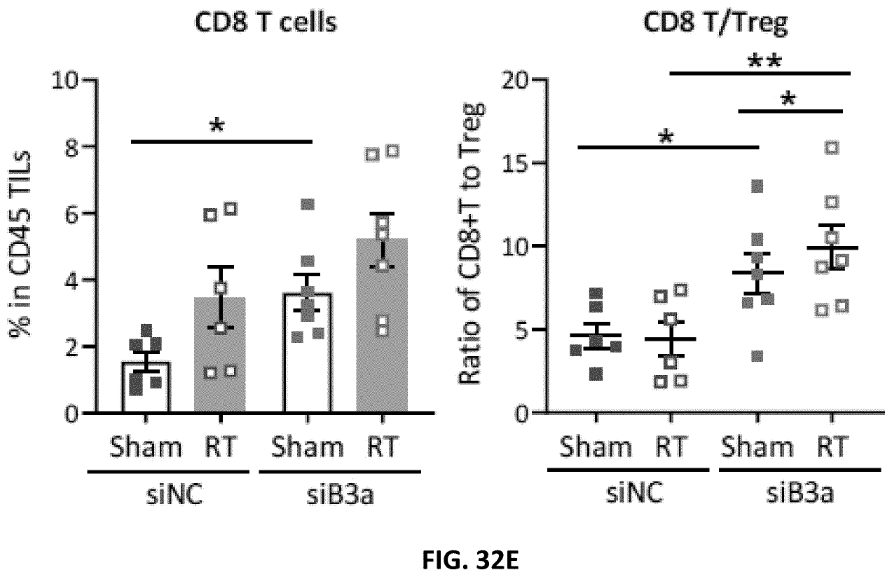

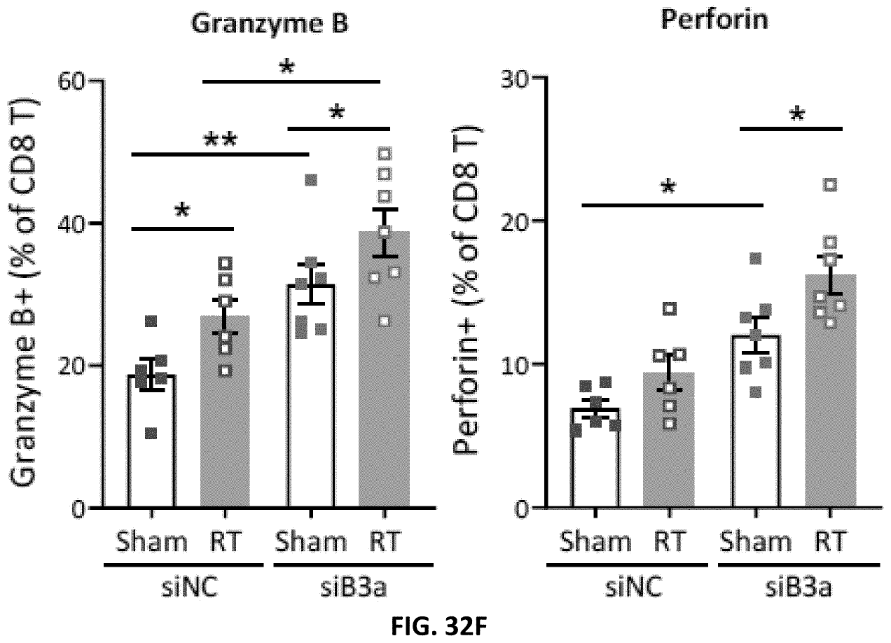

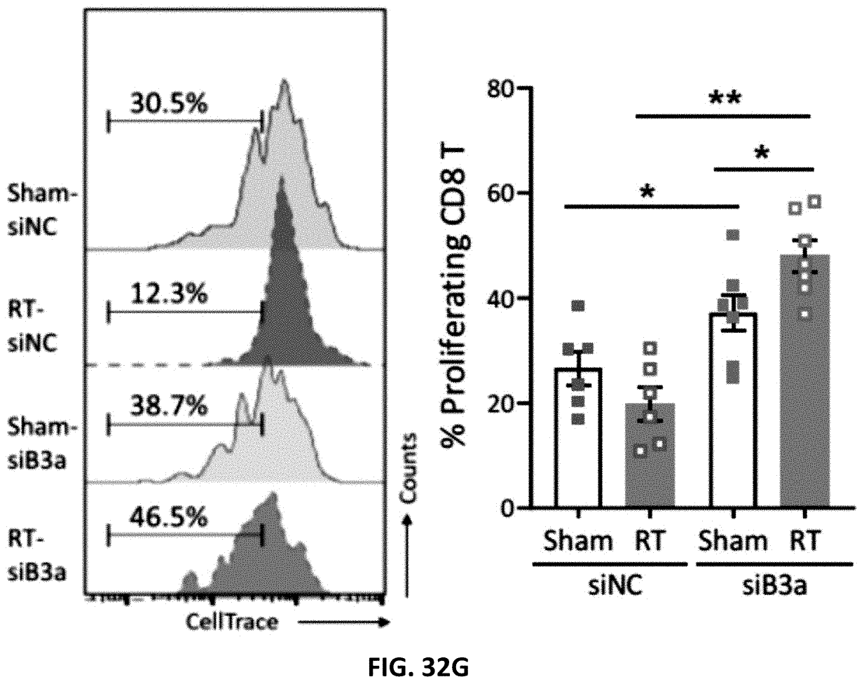

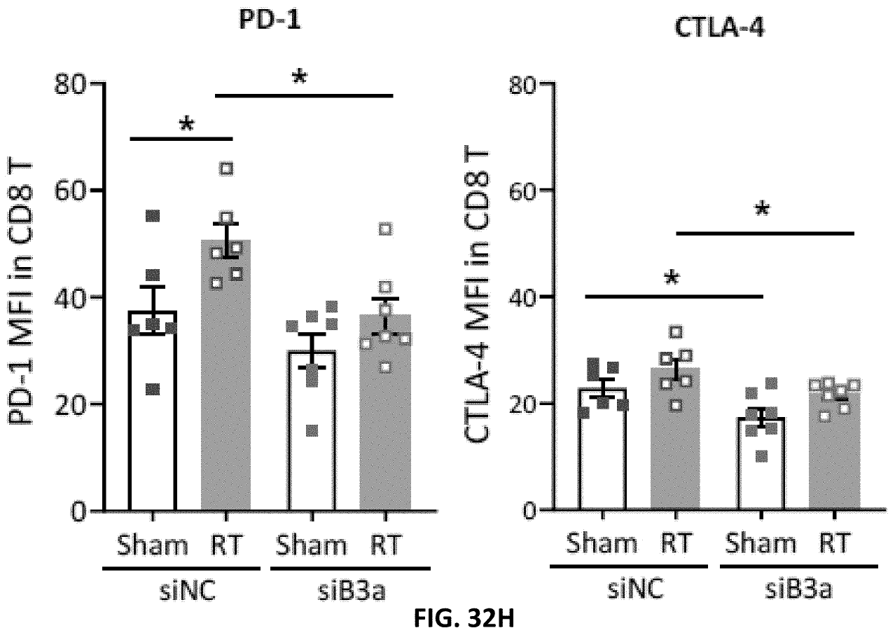

It was sought to understand the potential of targeting SERPINB3 in tumor growth inhibition and whether combination with radiation therapy could enhance antitumor immunity. To this end, LL2/B3a tumors were treated with Serpinb3a siRNA (siB3) or negative control siRNA (siNC) on day 9 post-tumor inoculation with repeated injections every 2-3 days and a single dose of 10 Gy or sham RT was given on day 14. The knockdown of Serpinb3a in tumors was confirmed by RNA expression, which showed an average of 65% decrease in siB3-treated tumors (FIG. 32A). Reduced tumor growth was observed in sham siB3-treated tumors compared to sham siNC-treated tumors, and the combination of Serpinb3a knockdown with RT (RT/siB3) resulted in more significant tumor growth inhibition (FIG. 32B). The high expression of CXCL1, and S100A8/A9 in LL2/B3a tumors showed a significant decrease by Serpinb3a knockdown (sham/siNC vs. sham/siB3). RT induced S100A8/A9 in both RT/siNC and RT/siB3 tumors but not CXCL1, which showed induction only in RT/siNC tumors, compared to their sham counterparts (FIG. 32C). Reduced suppressive chemokine secretions in siB3 tumors might lead to decreased myeloid cell infiltration. Indeed, sham/siB3 tumors demonstrated a significant reduction in M-MDSCs, PMN-MDSCs, and M2 macrophages compared with sham/siNC tumors. Increased PMN-MDSCs after radiation was seen in both RT/siNC and RT/siB3 tumors whereas increased M-MDSCs were only observed in RT/siNC tumors (FIG. 32D). In addition, radiation-induced antitumor immunity relies on functional cytotoxic T cells infiltration. An increase in CD8+ T cells was observed in sham/siB3 tumors and RT seemed to have effects on promoting CD8+ T cell infiltration as no statistical difference was noted between RT/siNC and RT/siB3 tumors. However, a higher ratio of CD8/Treg in sham/RT siB3vssiNC tumors, as well as an increased ratio in RT/siB3 vs sham/siB3, suggested reduced Treg infiltration by Serpinb3a knockdown and that RT-induced CD8+ T cell infiltration was accompanied by Treg expansion in siNC but not siB3 tumors (FIG. 32E). CD8+ T cells in siB3 tumors also demonstrated improved cytotoxic potential with increased expression of granzyme B and perforin, which were further enhanced by radiation treatment (FIG. 32F). Similarly, improved T cell activity upon anti-CD3/28 stimulation was observed in siB3 tumor-derived CD8+ T cells, which demonstrated markedly higher proliferative capacity than those derived from siNC tumors (FIG. 32G). This correlated with lower PD-1 in RT/siB3 vs RT/siNC and CTLA-4 in sham/RT siB3 vs siNC tumors, indicating less exhausted CD8+ T cells when silencing Serpinb3a in tumors (FIG. 32H). Collectively, targeting Serpinb3a resulted in remolding infiltrating myeloid cells and reduction of immunosuppressive chemokines, together with enhanced T cell function, and when combining Serpinb3a knockdown and radiation therapy, it achieved more significant inhibition in tumor progression and improved radiation-induced antitumor immunity.

SERPINB3 Mediates Suppressive Chemokine Production by Promoting STAT Activation

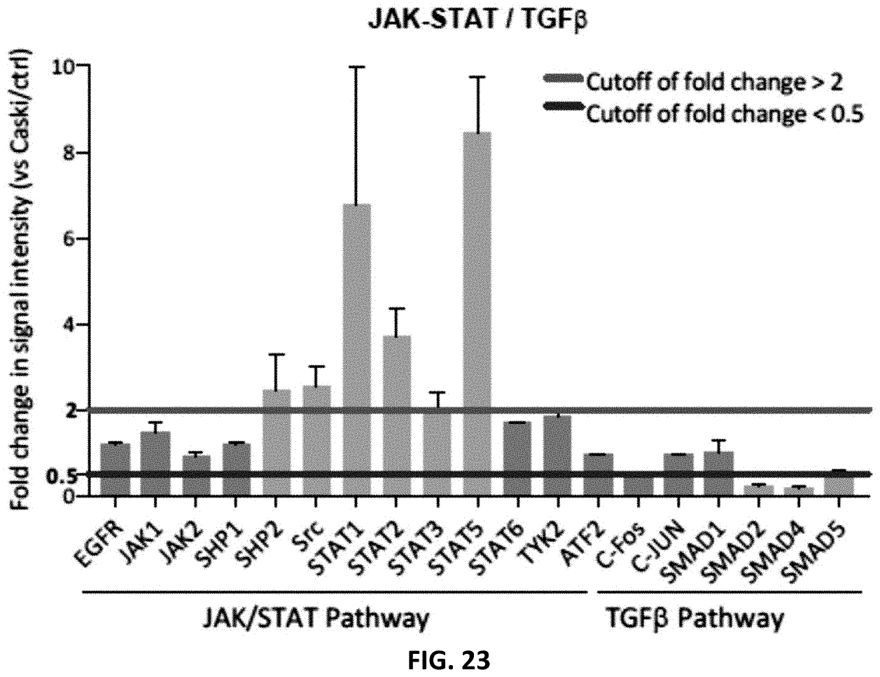

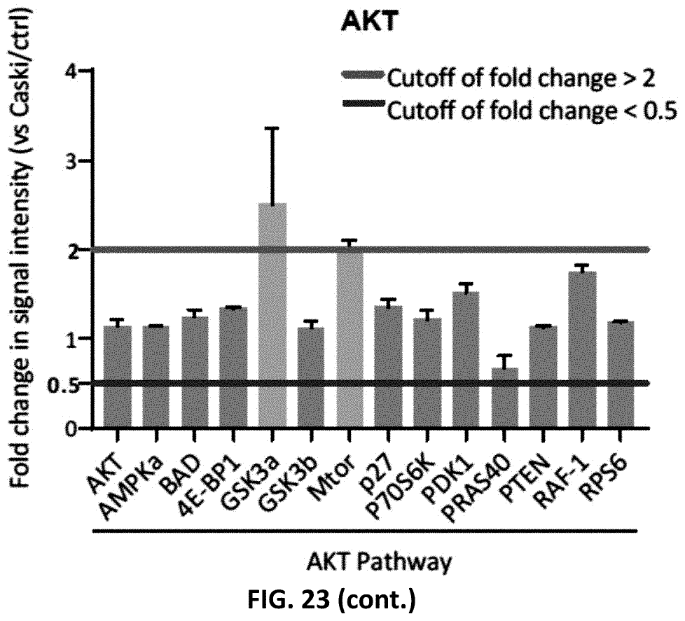

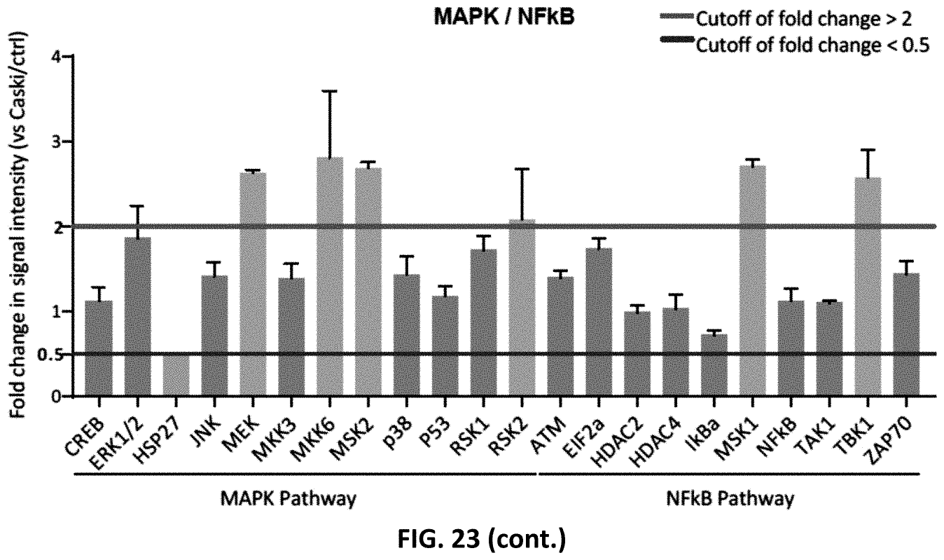

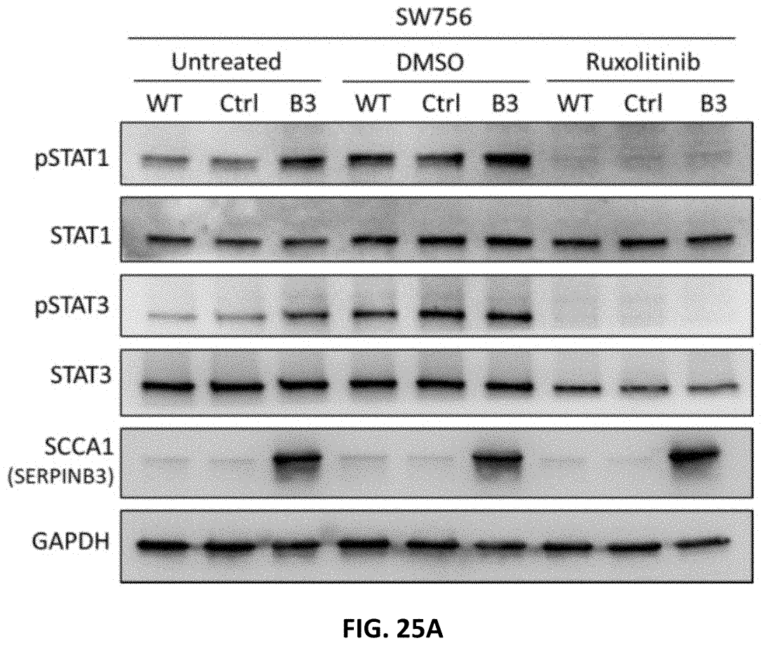

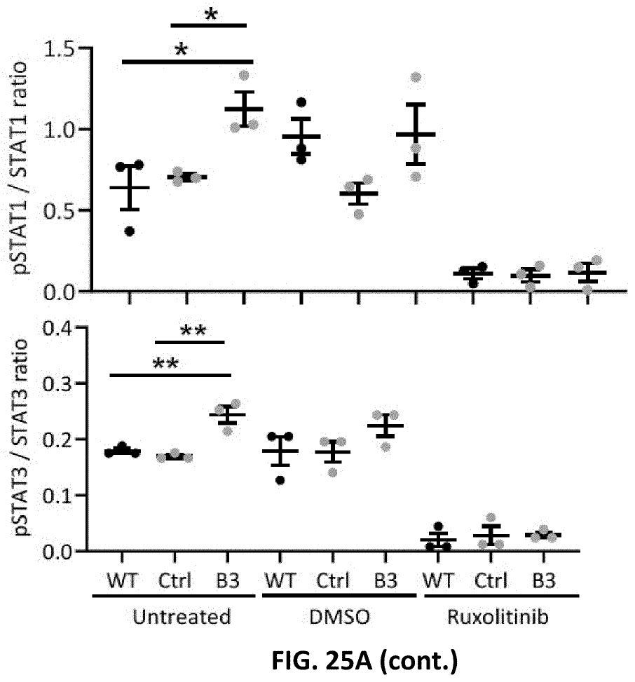

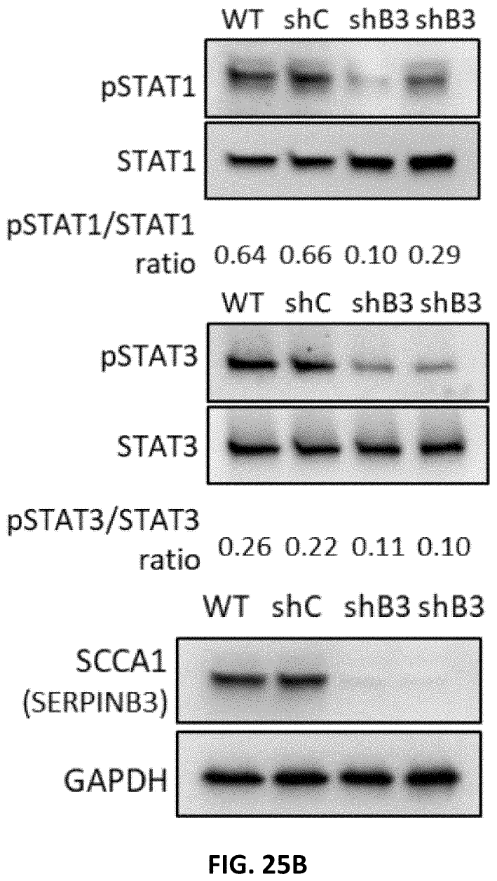

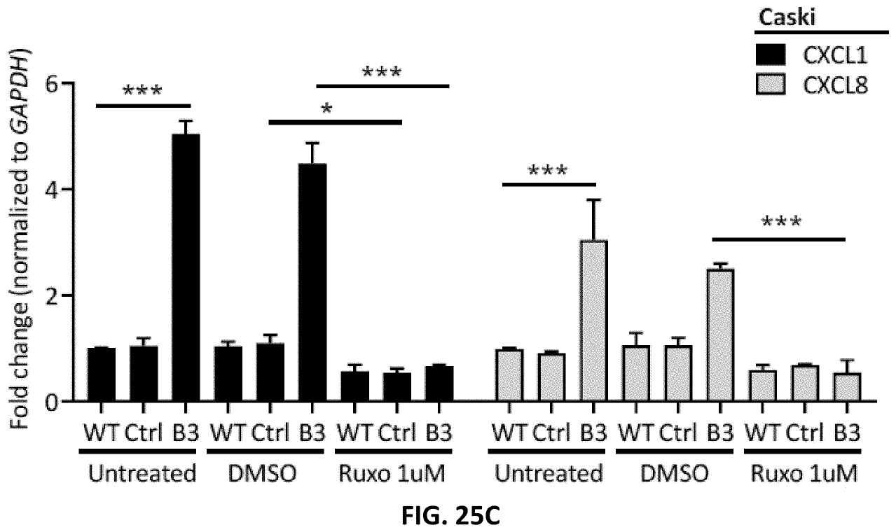

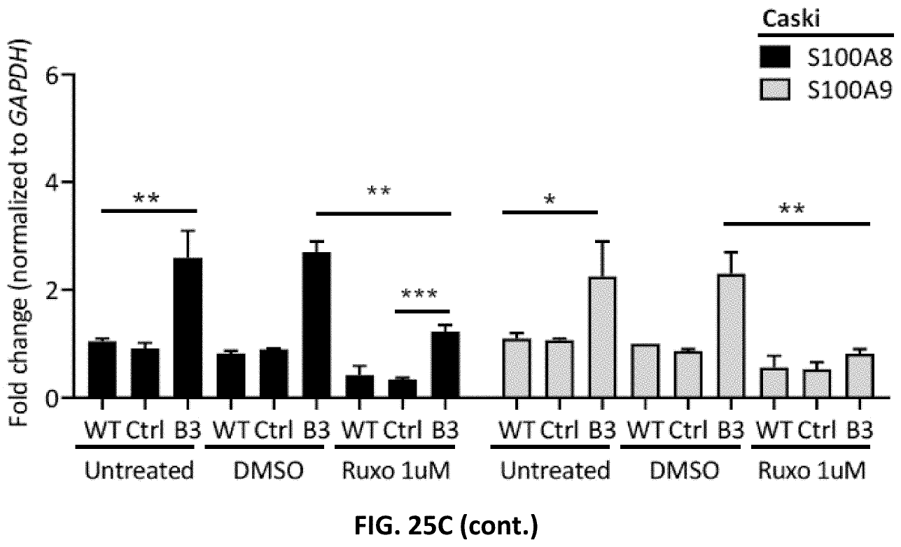

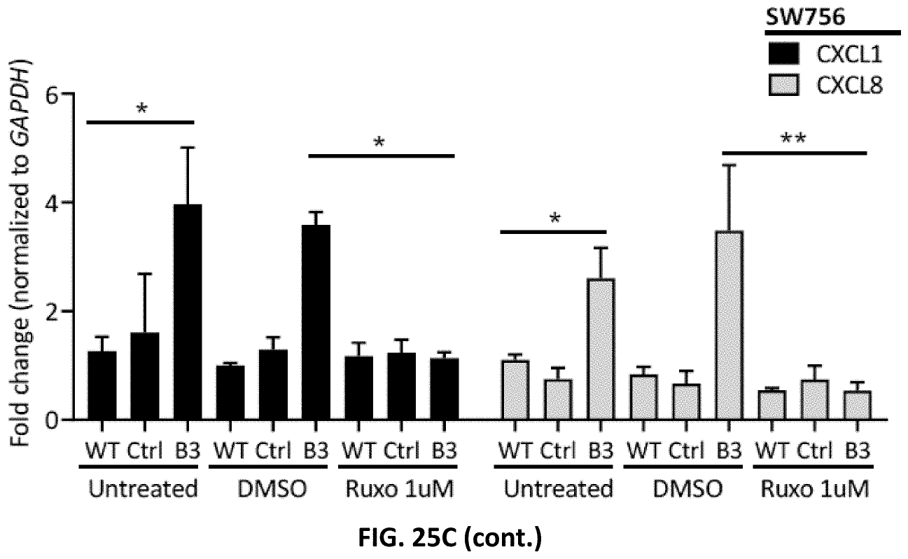

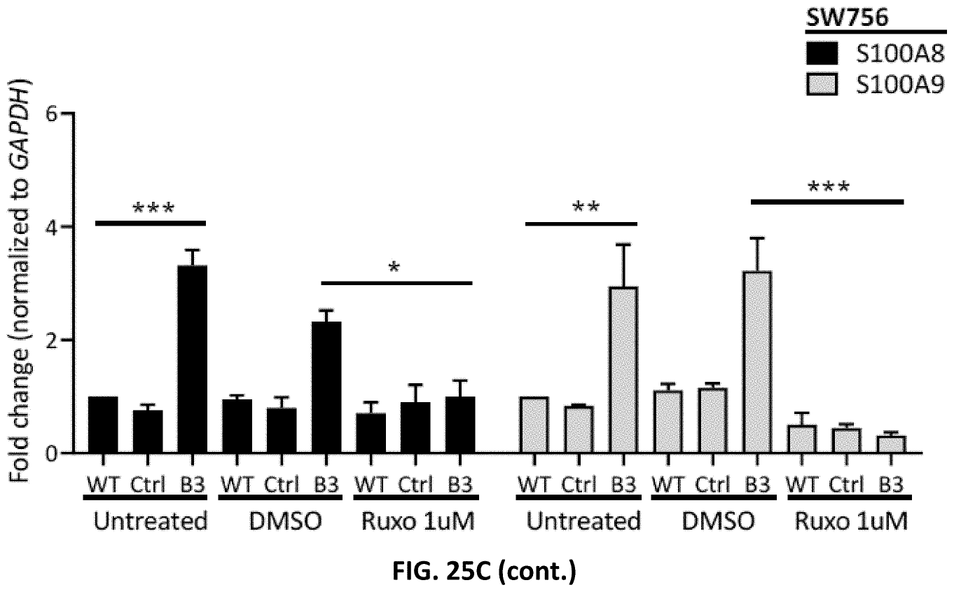

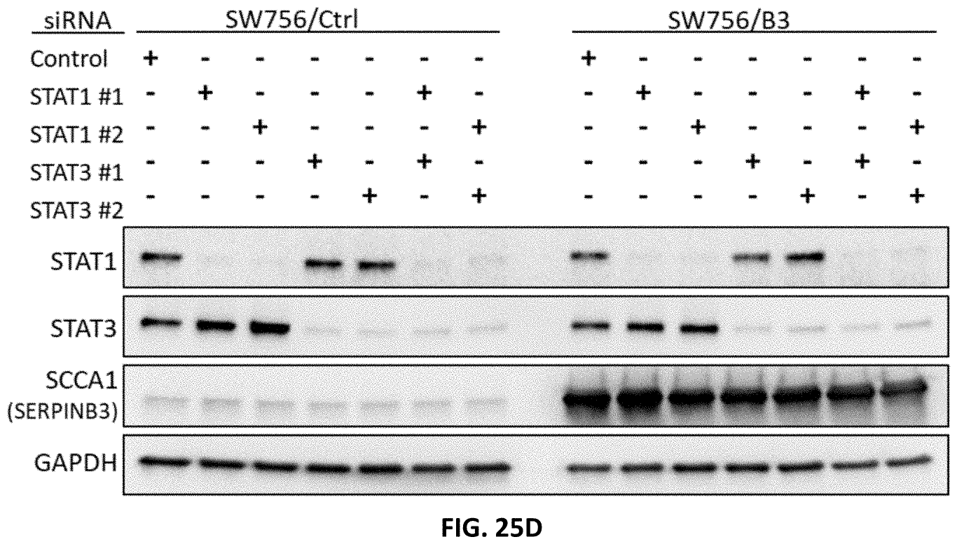

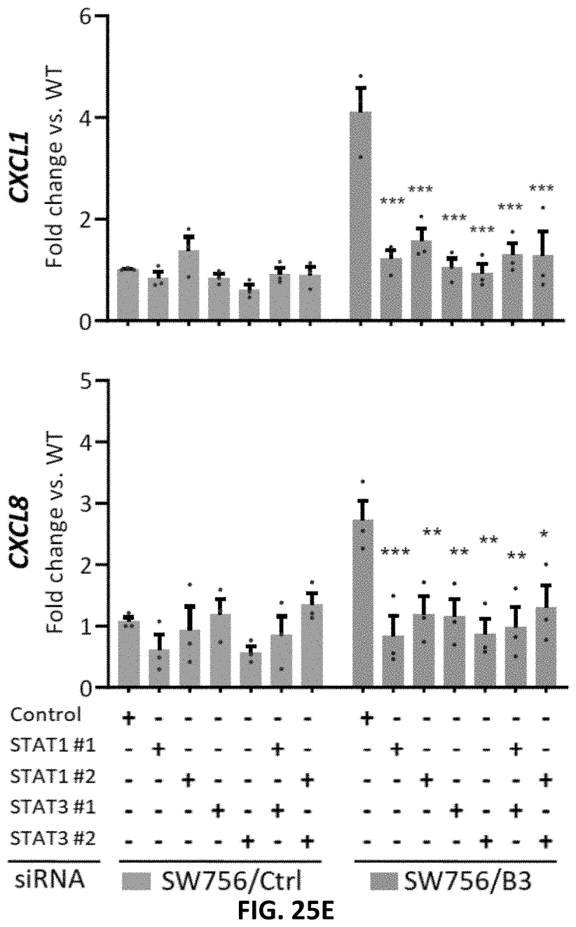

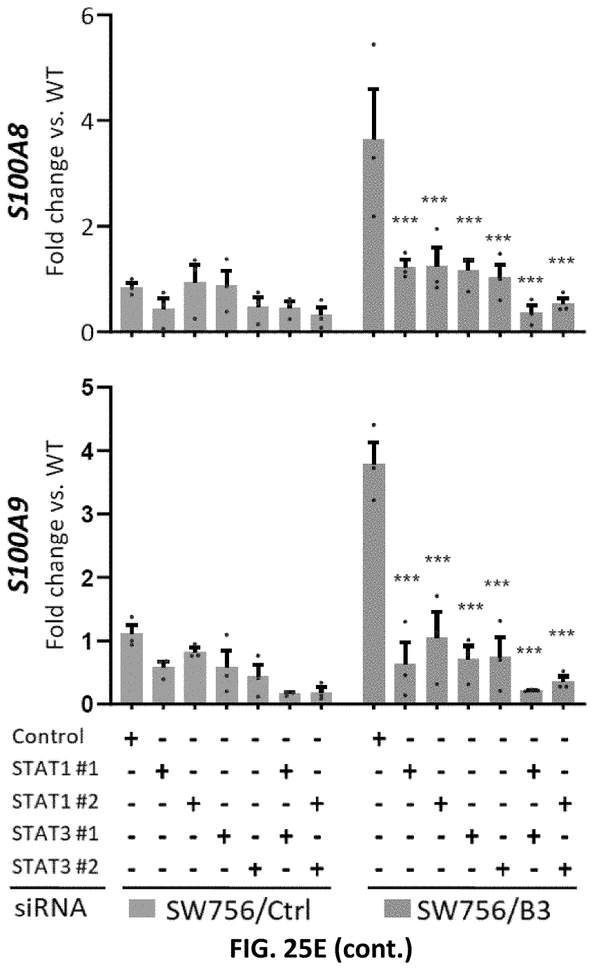

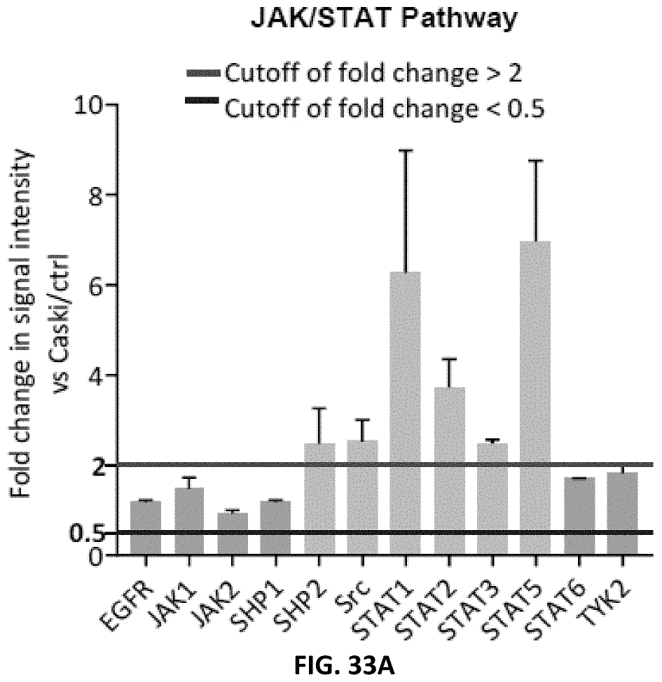

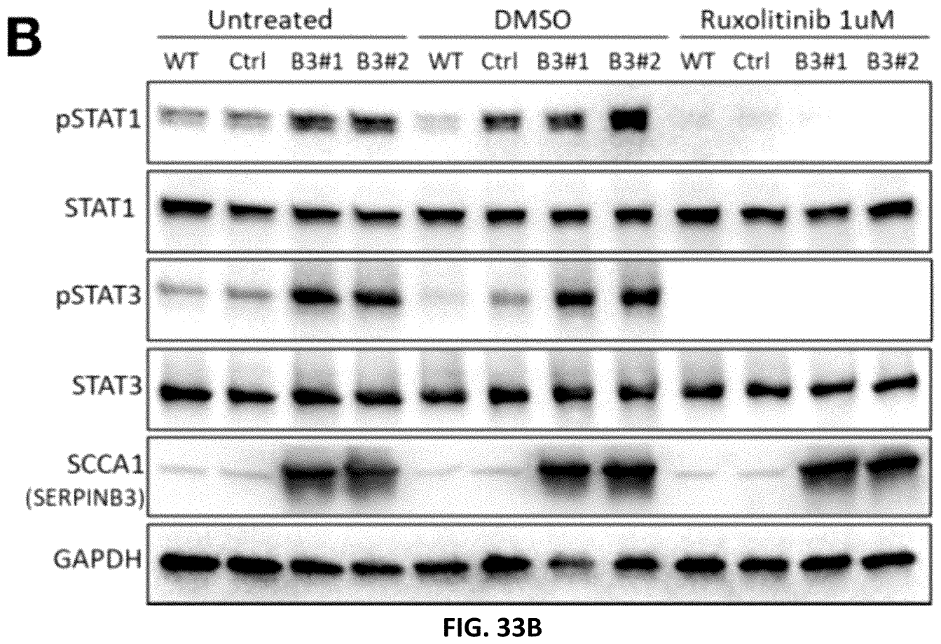

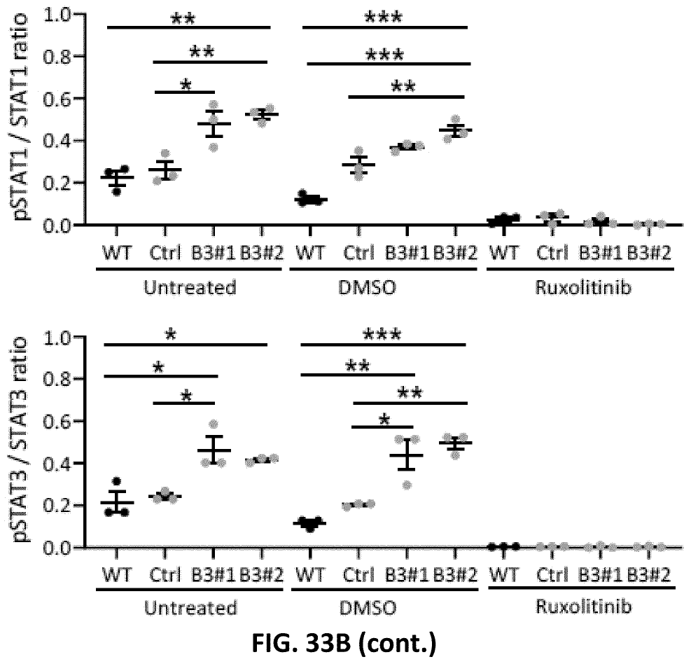

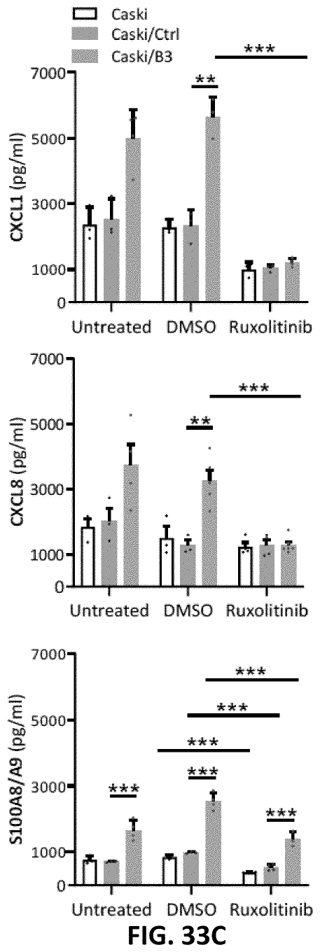

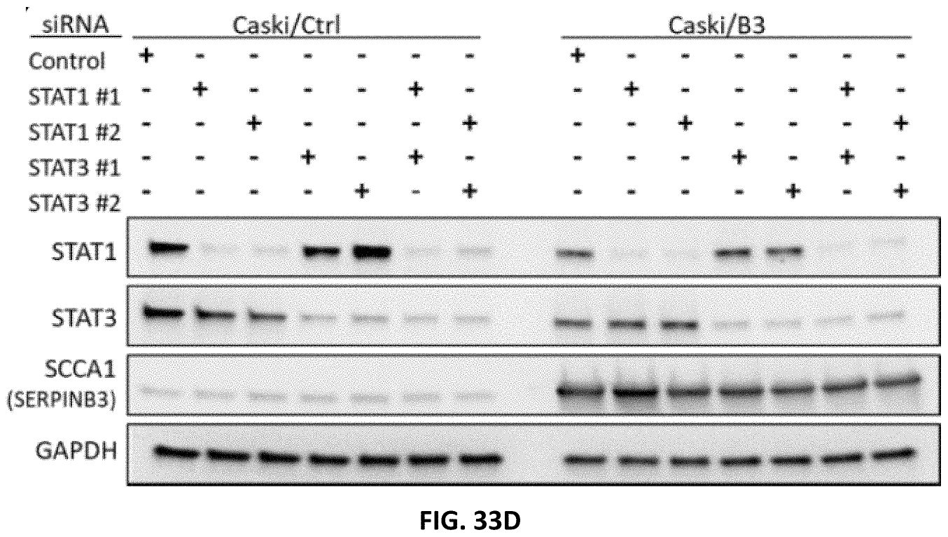

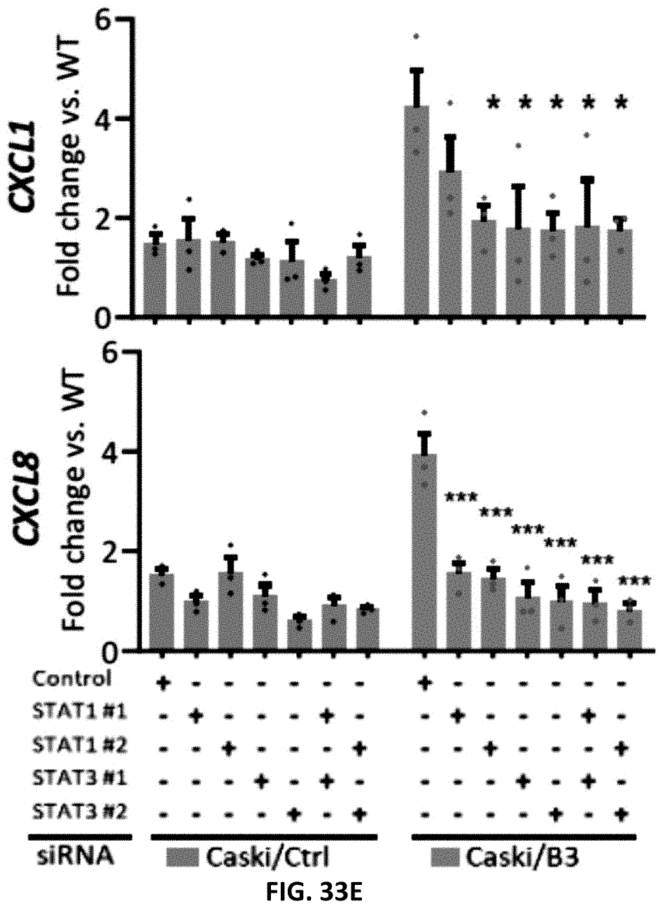

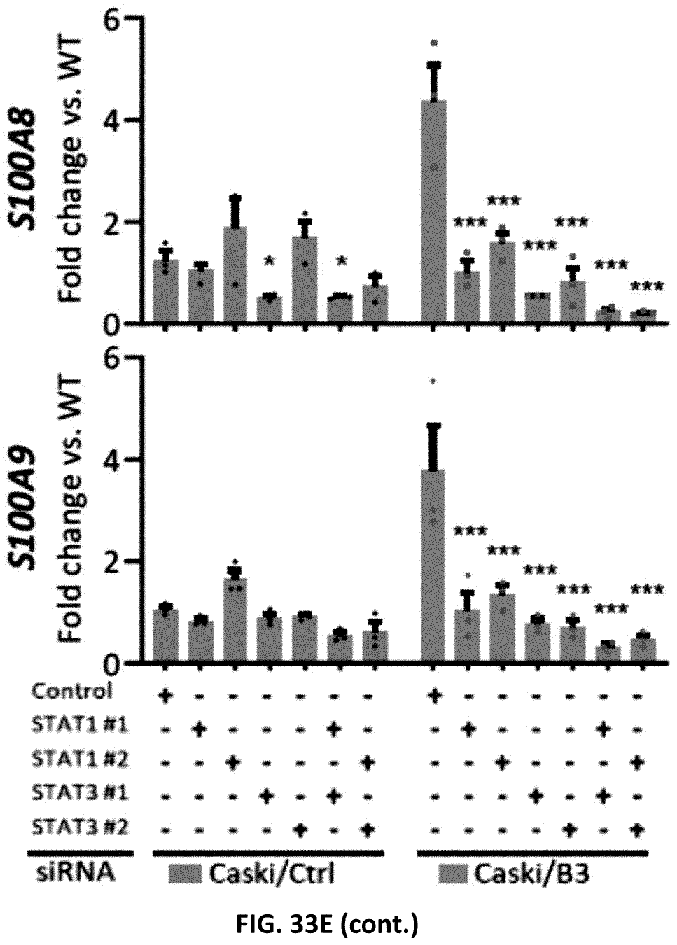

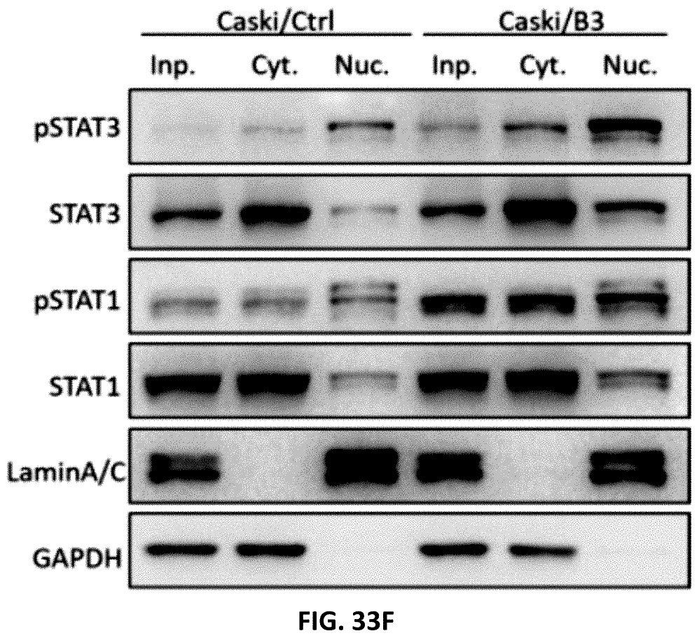

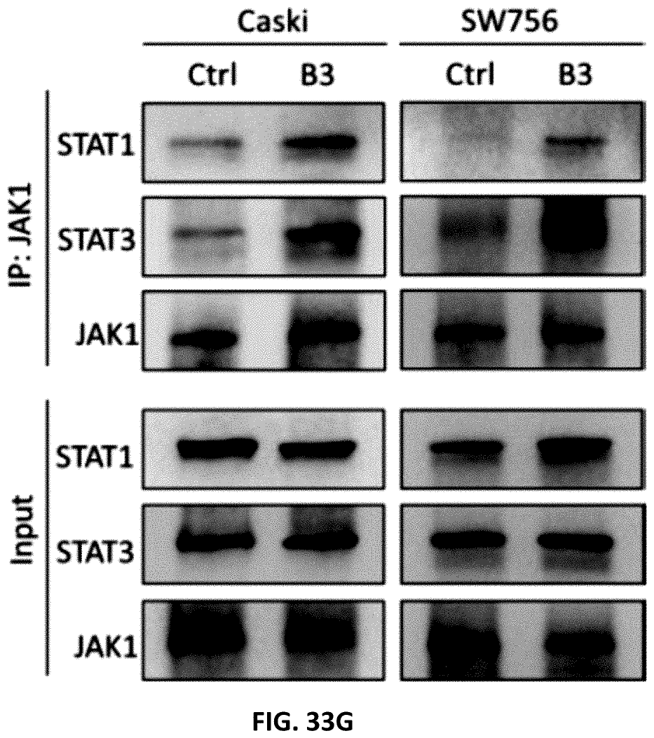

Although SERPINB3 has been implicated in pro-inflammatory signaling in pancreatic cancer and Kras mutant tumors, the underlying molecular mechanism is unknown. To provide further insight into SERPINB3-mediated suppressive immune response, we employed a human phosphorylation pathway profiling array that contained five cancer-associated pathways—MAPK, AKT, JAK/STAT, NF-κB, and TGF-β, and identified 14 proteins with upregulated phosphorylation (fold change≥2) and 4 proteins with downregulation (fold change≤50.5) in Caski/B3 compared to Caski/Ctrl cells (FIG. 23). Among those with increased phosphorylation, signal transducer and activator of transcription (STATs) proteins, including STAT1/2/3/5, showed the highest magnitude of change in phosphorylation (FIG. 33A). Phosphorylation of STAT1 and STAT3 in response to SERPINB3 expression was examined in Caski and SW756 cells, where the induction of SERPINB3 resulted in increased pSTAT1 and pSTAT3 (untreated, FIGS. 33B and 25A). In contrast, the knockdown of SERPINB3 by shRNA led to reduced pSTAT1 and pSTAT3 expression (FIG. 25B). Given that STAT proteins regulate the transcriptional activity of many cytokines and chemokines, it was hypothesized that SERPINB3 mediates suppressive chemokine production through promoting STAT signaling activation. FDA-approved small molecule inhibitor, ruxolitinib, inhibiting the phosphorylation of STAT1 and STAT3 was confirmed by immunoblotting (FIGS. 33B and 25A). The initially high secretion of CXCL1/8 and S100A8/A9 in Caski/B3 and SW756/B3 cells was significantly suppressed by ruxolitinib treatment, suggesting an essential role of STAT activation in chemokine production in SERPINB3 cells (FIGS. 33C and 25C). To further understand whether STAT signaling directly regulated chemokine expression in SERPINB3 cells, siRNA was used to individually silence STAT1 or STAT3 (FIGS. 33D and 25D). The expression of CXCL1/8 and S100A8/A9 was decreased in SERPINB3 cells by silencing either STAT1 or STAT3, and the simultaneous knockdown of both STAT1 and STAT3 did not lead to more significant suppression in CXCL1 and CXCL8. However, the knockdown of both STAT1 and STAT3 achieved more effective inhibition of S100A8 and S100A9 in SERPINB3 cells (FIGS. 33E and 25E). Moreover, both STAT1 and STAT3 proteins showed increased phosphorylation and nuclear translocation in SERPINB3 cells, indicating upregulated transcriptional activity in promoting downstream gene expression (FIG. 33F). Notably, an increase in phosphorylated STAT1/3 was not only observed in the nucleus but also cytoplasm, which suggests SERPINB3 may be involved in mediating upstream cytoplasmic kinase of the signaling cascade to promote STAT activation. Thus, co-immunoprecipitation of JAK1 protein was performed and increased interaction of JAK1 with STAT1 and STAT3 in Caski/B3 and SW756/B3 was found compared to Caski/Ctrl and SW756/Ctrl cells (FIG. 33G). These data show that suppressive chemokine production in SERPINB3 cells relies on STAT signaling and SERPINB3 mediated STAT activation through promoting JAK/STAT interaction, leading to increased STAT transcriptional activity.

Elevated Serum SCCA and High Tumor pSTAT3 are Associated with CD11b Expression and Poor Cancer-Specific Survival after CRT

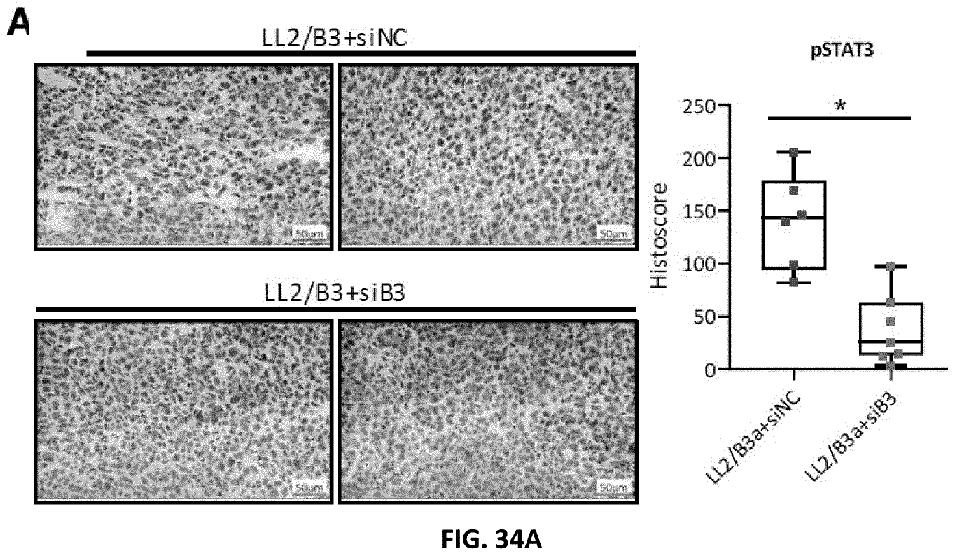

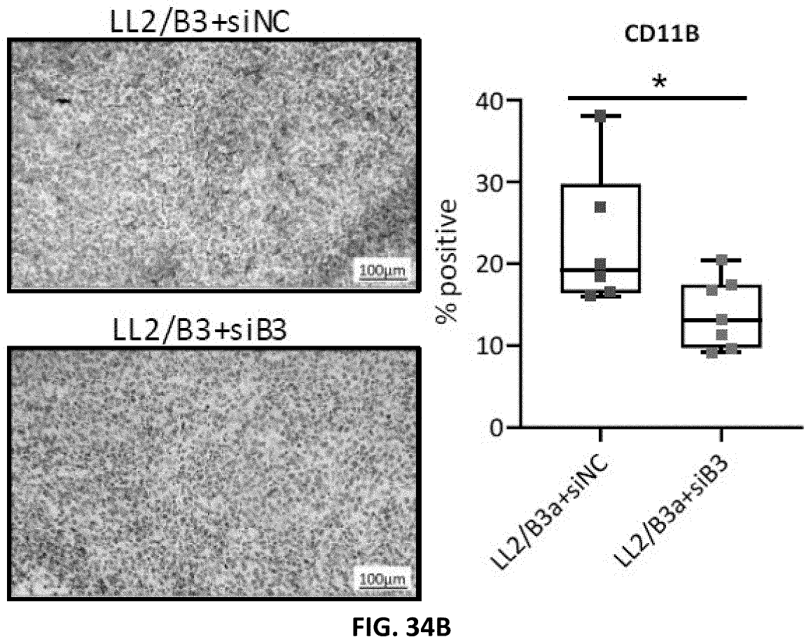

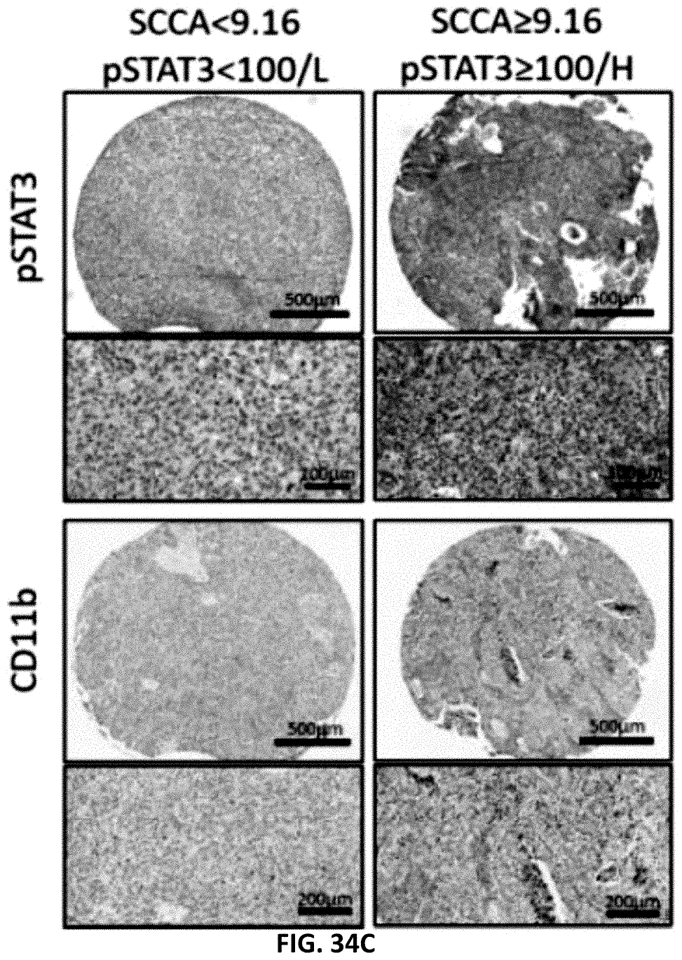

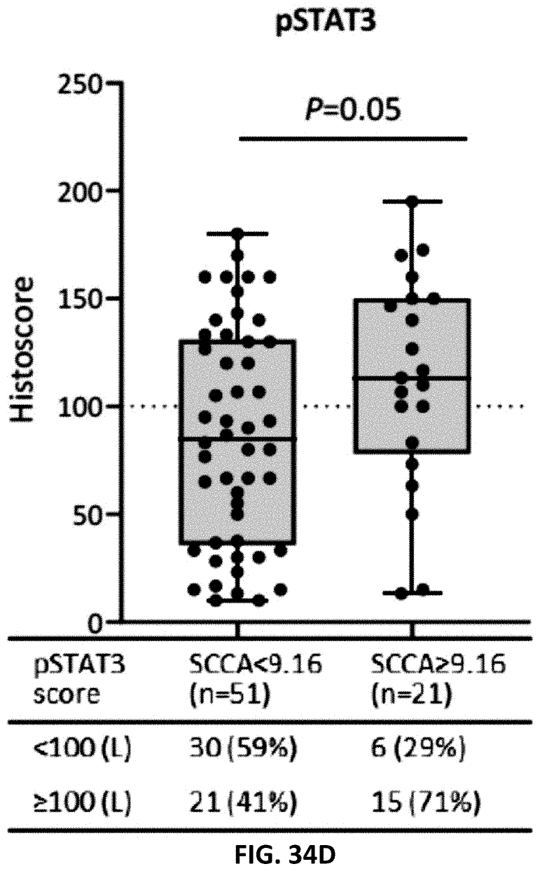

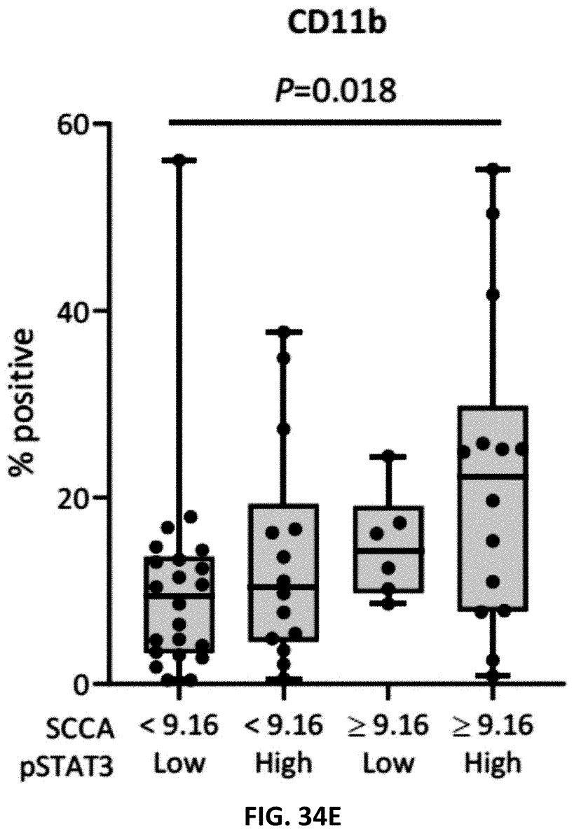

In corresponding to in vitro findings, mouse LL2/B3a tumors with initially high pSTAT3 expression were significantly reduced by the intratumoral knockdown of Serpinb3a (LL2/B3a+siB3), evident by the immunostaining of pSTAT3(Tyr705) (FIG. 34A). The reduction of pSTAT3 in Serpinb3a knockdown tumors further correlated with reduced CD11b+ myeloid cell infiltration (FIG. 34B).

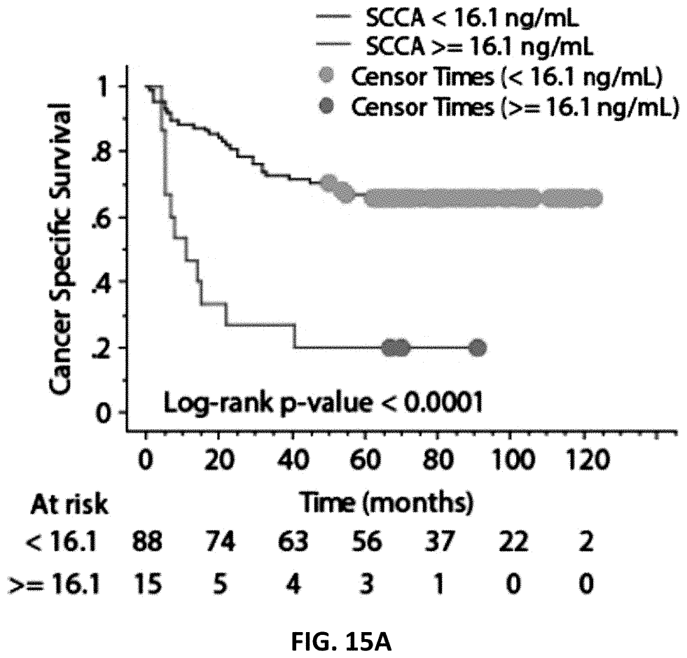

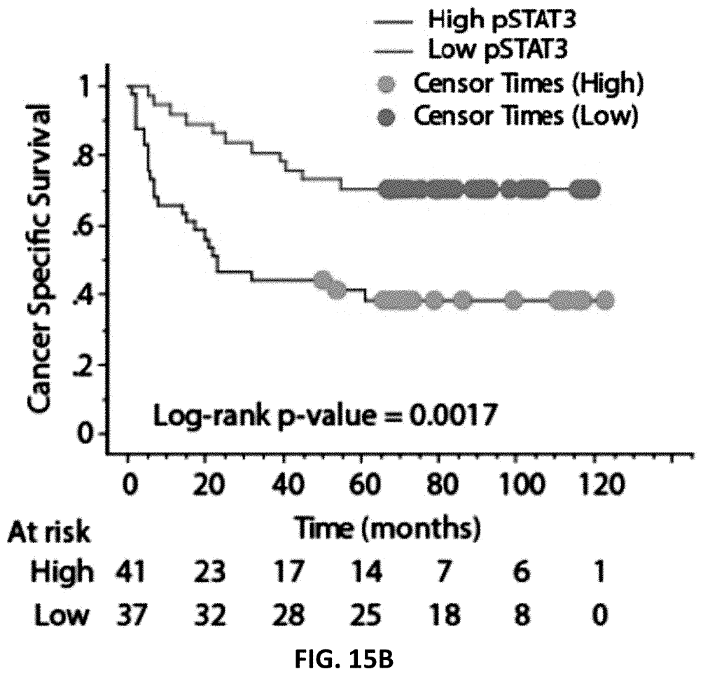

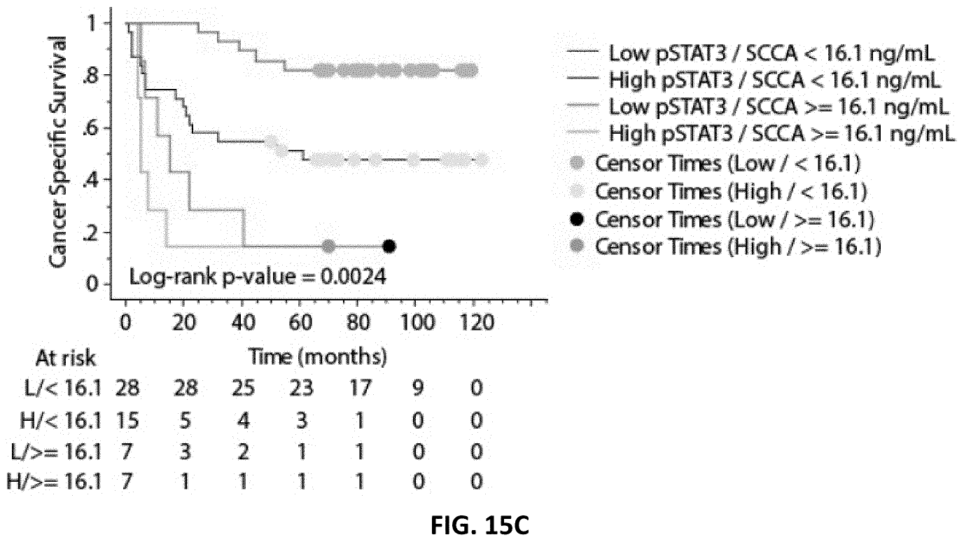

To evaluate the clinical implications of these findings, tissue microarrays containing pre-treatment cervix tumor biopsy specimens obtained from patients with biopsy-proven invasive cervical carcinoma were immunostained for pSTAT3 (Tyr705) and myeloid cell marker CD11b. Pretreatment serum SCCA values from 72 cancer patients with an average of 9.16 ng/ml presented a significant cutoff point for cancer-specific survival in our patient population. Patients with elevated pretreatment SCCA (≥9.16 ng/ml) had worse survival than those with low SCCA at the time of diagnosis (FIG. 15), in agreement with a previous study reporting SCCA as a clinical biomarker. The histoscore of pSTAT3 evaluated by immunochemistry was determined through the combined factors of the intensity and percentage of stained cells within the tumor proportion of TMA cores using an attribute cutoff of 100 for high or low pSTAT3 expression (FIG. 34C). In high pretreatment SCCA patient cohort (≥9.16 ng/ml), 71% of the population showed high pSTAT3 histoscore as opposed to 41% of high pSTAT3 in low pretreatment SCCA patients (<9.16 ng/ml) (FIG. 34D). Although pSTAT3 was not an independent prognostic factor for survival in our cohort, the patients with elevated serum SCCA, along with high pSTAT3 were associated with increased CD11b expression (FIG. 34E). In contrast, the majority of patients with pretreatment SCCA<9.16 ng/ml had low pSTAT3 histoscore and was correlated with low CD11b expression. This cohort had the highest cancer-specific survival (FIG. 15). Overall, SCCA is a strong clinical biomarker, and when combined with pSTAT3 expression, it may indicate an unfavorable tumor microenvironment and provide an opportunity for selection of patients for anti-STAT and/or anti-SERPINB3 directed therapies.

### Discussion

In this study, it was revealed that SERPINB3 modulated TME towards an immunosuppressive phenotype by upregulating CXCL1/8 and S100A8/A9 production, to facilitate tumor growth and impede the success of RT. These chemokines were increased in the tumors of both human SERPINB3-expressing Caski xenograft and murine Serpinb3a-expressing LL2 syngeneic mouse models, resulting in substantial numbers of infiltrating M-MDSCs, PMN-MDSCs, and M2 macrophages. Radiation-induced T cell responses were compromised by the suppressive microenvironment in SERPINB3 tumors. Targeting SERPINB3 in tumors reshaped TME, which demonstrated reduced suppressive chemokine production and myeloid cell infiltration, leading to enhanced T cell activity. More importantly, targeting SERPINB3 in combination with RT showed significant tumor growth inhibition and improved RT-induced T cell immunity. It is worth noting that the correlation between SERPINB3 and CXCL1/8, S100A8/A9 was conserved across several cancers known to have high SERPINB3 expression and often associated with poor treatment outcomes, suggesting a wide potential application of these results to a variety of tumors with SERPINB3 expression.

The association between SERPINB3 and chemokines has been reported in atopic dermatitis and psoriasis, whereby downregulation of SERPINB3 in keratinocytes was associated with reduced expression of CXCL1, 5, 8, and S100A8. Catanzaro and colleagues showed that SERPINB3 was a downstream mediator of mutant Ras-induced tumorigenesis and knockdown of SERPINB3 led to decreased IL-6, CXCL1, and CXCL8 production suppressing tumorigenesis. However, the downstream immune consequences were not further examined in these studies. CXCL1/8 are potent mediators of immune cell chemotaxis via chemokine receptor CXCR2, commonly expressed on myeloid progenitor cells, neutrophils, monocytes, and macrophages. S100A8 and S100A9 are well-established immunosuppressive factors in tumors, known to attract MDSC infiltration, promote suppressive macrophage differentiation, and stimulate immunosuppressive activity. Given the crosstalk between chemokines and immune cells, it was revealed that the secretion of CXCL1/8 and S100A8/A9 by SERPINB3-expressing tumors resulted in increased suppressive myeloid cell infiltration, including M-MDSCs, PMN-MDSCs and M2 macrophages. These suppressive myeloid cells have been shown to promote tumor progression by inhibiting antitumor immunity and disrupting T cell activation signaling, as well as facilitating tumor angiogenesis and neoplastic cell invasion and forming a pre-metastatic niche. In patients with cervical cancer and esophageal squamous cell carcinoma, high expression of SERPINB3 was associated with lymph node metastasis; however, the underlying causes are unknown. The increased immunosuppressive myeloid cell populations provided a possible mechanism for the high metastatic tendency of SERPINB3 tumors and identified a potential target for tumor control.

Strategies of targeting myeloid cells to improve T cell-mediated immunity or alter myeloid cell polarization and infiltration have been studied extensively. For instance, CXCR1/2 and CCR2 inhibitors interrupted CXCL8/CXCR1-2 and CCL2/CCR2 axis, blocking the recruitment of TAMs and MDSCs. Colony-stimulating factor 1 receptor (CSF-1) inhibitor repolarized TAMs from M2-like to M1-like phenotype and depleted TAMs to reduce suppressive immune responses but studies also found increased PMN-MDSC infiltration through a CXCR2-dependent manner. Other compensatory actions, such as the expansion of monocytes and macrophages when targeting granulocytes, and compensatory upregulation of PD-L1 and CTLA-4 by untargeted myeloid cells have also been reported. This limits the therapeutic efficacy of myeloid cell-targeting strategies. Similarly, when we depleted CD11b+ myeloid cells in SERPINB3 tumors, initial tumor growth inhibition and improved T cell activities were observed. However, with tumor progression, no difference in tumor doubling time between CD11b-depleted and non-depleted tumors was observed and an immune cell population with intermediate levels of CD11b expression appeared in CD11b-depleted tumors was also found. This suggests a combined therapy or additional strategies to overcome compensatory mechanisms triggered by myeloid cell-targeting therapies might be required.

Hyperactivated STAT3 signaling has been shown to mediate immunosuppression through tumor cell intrinsic and extrinsic mechanisms, and is associated with poor clinical prognosis in many cancers, including cervical, lung, and head and neck cancer, where elevated SERPINB3 expression was commonly observed. STAT3 regulates the expression of genes involved in tumor cell survival, proliferation, and angiogenesis, while with the increasing interest in immunotherapy, more and more studies have demonstrated that STAT3 also mediated the crosstalk between tumor and immune cells. For instance, hyperactivated STAT3 inhibited the production of cytokines/chemokines critical for adaptive immunity, induced the expression of PD-1/PD-L1, as well as suppressed T cell-mediated anti-tumor immunity. Moreover, increased STAT3 activity has been implicated in suppressive immune cell population such as Tregs and MDSCs; therefore, the potential of inhibiting STAT activity to improve therapeutic responses have been explored in many preclinical studies. Ruxolitinib, the first FDA-approved JKA/STAT pathway inhibitor, successfully triggered tumor regression, increased drug sensitivity, and prevented angiogenesis in many mouse models of cancers. However, clinical trials of pancreatic adenocarcinoma, breast cancer, colorectal cancer, and lung cancer showed lack of efficacy and very limited or no overall survival benefit. Among a few other ongoing trials, a phase I study in glioblastoma (NCT03514069) showed a promising preliminary result from combing ruxolitinib with radiation and temozolomide. The consideration of using STAT inhibitor in combination with radiation was also found in a recently completed trial (NCT01904123) using STAT3 inhibitor—WP1066, for patients with recurrent malignant glioma. Their preclinical study showed that STAT3 inhibitor and irradiation reprogrammed immunosuppressive glioma TME by improving dendritic cell maturation and interactions with T cells, resulting in enhanced survival compared to either treatment alone. Of note, HPV-related cancers, including cervical and head and neck, often showed hyperactivated STAT3 due to virus-associated inflammatory responses. Ruxolitinib demonstrated in-vitro effects on facilitating Cisplatin-induced cell death in HPV-positive cervical cancer cells; however, in vivo efficacy has not been investigated and whether this success can transition into clinical trials remain unclear.

In addition, knowing that STAT transcriptional activity is also involved in T-cell function, and other facets of the immune response, direct inhibition of this pathway may unintentionally tip the immune axis back in favor of the tumor. Here, the rationale for targeting an upstream, tumor-specific signal—SERPINB3, to be a more effective approach in the clinical setting is provided. Silencing SERPINB3 leads to the reduction in infiltrating immunosuppressive myeloid cells and in return, enhances T cell responses. The changes in TME further render radiation treatment effective in previously radioresistant SERPINB3 tumors. Nonetheless, we observed increased PD-1 and CLTA-4 expression in SERPINB3 tumors while targeting SERPINB3 slightly reduced the expression of CTLA-4 but not PD-1. The potential connection between SERPINB3 and immune checkpoints has also been reported in HPV-negative head and neck squamous cell carcinoma, where patients with high SERPINB3 expression corresponded to increased PD-L1 and PD-L2. Similarly, genome-level and IHC showed upregulated PD-L1 in SERPINB3-high ovarian and esophageal tumors. Although SERPINB3 knockdown improved cytotoxic T cell function, an increase in PMN-MDSCs and slightly higher PD-1 expression were observed following RT. Therefore, combined therapy targeting SERPINB3 and immune checkpoint inhibitors may reduce immunosuppressive chemokine-associated induction of myeloid cells and, on the other hand, may prevent T cell exclusion and dysfunction, leading to maximal RT-induced antitumor immunity.

The findings that SERPINB3 modulates the crosstalk between immune and cancer cells via secretion of CXCL1/8 and S100A8/A9 implicates this protease inhibitor member of the SERPIN superfamily in a key tumor strategy to evade the anti-tumor immune responses and resist therapies such as radiation. Targeting SERPINB3 reprograms the immunosuppressive environment and sensitizes the tumor to radiation therapy. This also presents several potential therapeutic combinations, such as STAT inhibitors or immune checkpoint blockade, to further improve treatment responses for cancers with elevated SERPINB3 expression.

### Methods

### Cell Lines and Plasmids

Cell lines were purchased from the ATCC. Caski, SW756, and C33a cells were cultured in Iscove's Modified Dulbecco's Medium (IMDM), and Lewis lung carcinoma cells (LL2/LLC) were cultured in Dulbecco's modified Eagle's medium (DMEM), supplemented with 10% fetal bovine serum (FBS), and 100 U/mL penicillin-streptomycin. The generation of SERPINB3 CRISPR-Cas9 knockout cells was described previously. SERPINB3 stable expression cells were generated using pULTRA lentiviral vector (Addgene #24129) containing human SERPINB3 or pLV-C-GFPSpark vector (Sino Biological LVCV-35) containing mouse Serpinb3a GFP-tagged fusion proteins. SERPINB3 knockdown cells were transduced with scramble shRNA (Addgene #1684) or SERPINB3 shRNA (Sigma Mission shRNA, TRCN0000052400). Genetically modified cells were generated through a lentivirus system by transfection of human 293T packaging cells. All cell lines were grown in monolayer at 37° C. with 5% CO₂ and periodically tested for Mycoplasma contamination.

### RNA Sequencing and TCGA Data Analysis

RNA sequencing (RNAseq) was performed on pre-treatment tumor biopsies obtained from patients enrolled in a prospective tumor banking study with written informed consent (Washington University IRB No. 201105374). RNAseq processing and normalization have been reported previously and data are available on GeneExpression Omnibus (GEO): accession number: GSE151666. Immune cell population and enrichment score were analyzed using xCell analysis, a gene signature-based method to estimate cell composition in bulk transcriptomic data. Heatmaps were generated using GraphPad Prism based on the average scores for each immune cell subtype in predefined patient groups. The CancerGenome Atlas (TCGA) RNAseq data were obtained through cBioPortal (https://www.cbioportal.org/). Spearman's correlation coefficient was used to test the significance of the correlation with an adjusted P<0.05.

### PBMC Isolation and Transwell Assay

Fresh primary PBMCs were obtained from patients who planned to undergo radiation therapy with brachytherapy for cervical cancer and had enrolled on a prospective biospecimen banking protocol (Institutional Review Board Protocol #201105374). A total volume of 10-20 ml of fresh blood was collected in EDTA separator tubes and peripheral blood mononuclear cells (PBMC) were immediately isolated using Lymphoprep and SepMate-50 (Stemcell Technologies) centrifugation tubes, according to the manufacturer's instruction. PBMCs were cultured in RPMI 1640 supplemented with 10% heat-inactive FBS and 100 U/mL penicillin-streptomycin. Transwell assay was performed using 8-μm Transwells (Falcon). Supernatants were collected from cells cultured in complete growth media for 48 h and loaded in the lower chamber of the transwell. PBMCs were loaded to the upper transwell for a 4 h migration period. Migrated cells were phenotyped by flow cytometry.

### Flow Cytometry and Data Analysis

Single-cell suspensions were blocked with either Human TruStain FcX Solution (422301, Biolegend) or mouse TruStain FcX PLUS (anti-mouse CD16/32) Antibody (S17011E, Biolegend) to avoid nonspecific Fc receptor binding and stained with LIVE/DEADFixable Dead Cell Stain Kit (MACS) to exclude dead cells. For surface staining, cells were incubated with the appropriate antibodies for 30 min at 4° C. Intracellular cytokine and nuclear staining was performed after surface staining using Cyto-Fast Fix/Perm Buffer Set and True-Nuclear Transcription Factor Buffer Set, respectively (BioLegend). Stained cells were analyzed using MACSQuant Analyzer 10 Flow Cytometer Antibody information is shown in FIG. 27. Data analysis including viSNE and FlowJo plugin FlowSOM were performed on FlowJo v.10 (TreeStar). A range of 20,000 to 60,000 live cells was acquired and individual flow cytometry data from each group were combined into a single data file for generating viSNE. Color-coded subpopulations were gated by pre-defined markers for each immune cell type and overlaid to the viSNE plots for total CD45+ cells from tumors. All flow cytometry gating plots, histograms, and statistics were generated on FlowJo.

Mouse Tumors with Anti-CD11b Antibody, Serpinb3a siRNA and/or Radiation Treatment

For xenograft models, female athymic nude mice aged 6-7 weeks old (Charles River) were injected subcutaneously with 5×106 Caski/Ctrl or Caski/B3 cells suspended in serum-free IMDM and 50% Matrigel Basement Membrane Matrix (Corning) to a final volume of 100 μl on their flank. For immunocompetent models, female C57/BL6 mice aged 7-8 weeks (Charles River) were injected subcutaneously with 5×10⁵ LL2/Ctrl or LL2/B3a cells suspended in 100 μl of PBS into the right flank. To deplete myeloid cells, mice were treated with anti-CD11b antibody (Ultra-LEAF purified anti-mouse CD11b, Biolegend) or isotype IgG2b as control (Ultra-LEAF purified rat IgG2b, Biolegend). Antibodies were administered through intraperitoneal injection at the initial dose of 300 μg in 150 μl PBS on day 10 post-tumor inoculation, and a subsequent dose of 150 μg in 100 μl PBS every three days. To knock down Serpinb3a, mice received either mSerpinb3a siRNA (5′-ACAUCGAAUUUAACUUCAUtt-3′; 5′-AUGAAGUUAAAUUCGAUGUtt-3′; ID:s73336, Thermo) or control siRNA (Ambion in vivo negative control #1 Thermo) complexed with Invivofectamine 3 (Thermo Fisher) as per manufacturer's protocol. Mice received three intratumoral injections of 10 μg siRNA on days 9, 11, and 13 post-tumor inoculation before radiation treatment performed on day 14. On days 16 and 19, mice received 20 μg siRNA via intraperitoneal injection. For radiation treatment, mice were randomized to receive sham or 10Gy RT using the Xstrahl Small Animal Radiation Research Platform (SARRP) 200 (Xstrahl Life Sciences). Tumor volume was measured twice weekly and calculated by (length×width2)/2. For tissue dissociation, tumors were manually dissected and digested with 1 mg/ml Collagenase, 0.5 mg/ml hyaluronidase, and 10 mg/mL DNase I type IV (Sigma), and transferred to a tissue disaggregator (Medicon, BectonDickinson) using a CTSV (Medimachine II, BectonDickinson) and tissue homogenates were filtered through a 100 μm strainer. Spleens were dissociated by being pressed through a 70 μm filter. Animal work was approved by the Washington University Institute Institutional Animal Care and Use Committee (Protocol #20-0470).

### Suppression Assay and T Cell Proliferation

Intratumoral myeloid cells were isolated from dissociated tumors using MojoSort mouse CD11b selection kit, biotin anti-mouse Ly6C, biotin anti-mouse Ly6G, biotin anti-mouse F4/80 antibodies, and streptavidin nanobeads (Biolegend) through magnetic purification. Splenic T cells were isolated from non-tumor-bearing mice and labeled with CellTrace Violet (Thermo Fisher) to evaluate proliferation. Purified myeloid cells were co-cultured with anti-CD3/CD28-activated T cells at a ratio of 1:1 for 4 days. The suppression was determined by CellTrace dilution using FACS to compare the proliferation of anti-CD3/CD28-activated T cells with and without myeloid cell co-culturing.

### Ex Vivo T Cell Stimulation

T cells isolated using MojoSort mouse CD3 T cell isolation kit (Biolegend) were labeled with CellTrace violet and activated with CD3/CD28 Dynabeads (Thermo Fisher) for 4 days to evaluate proliferation. To examine TNF and IFNr, T cells were stimulated with 500× Cell Activation Cocktail containing 40.5 μM phorbol-12-myristate 13-acetate (PMA) and 669.3 μM ionomycin (Biolegend) in the presence of 5 μg/mL BFA (Biolegend) for 5 h and stained with surface/intracellular markers for FACS analysis.

### Serum SCCA and Tissue Microarray Immunohistochemistry

Pretreatment serum SCCA was evaluated by ARUP National Reference Laboratory (Salt Lake City, UT, USA) using ELISA, and tissue microarrays were generated from untreated human tumor specimens. TMA sections were sent to HistoWiz Inc for IHC staining for CD11b (1:100, Abcam ab224800) and IHC for pSTAT3 (1:200, Sigma SAB4300033) was performed by Washington University AMP Core Labs. Mouse tumor sections were stained with pSTAT3 (1:150, Invitrogen PA5-121259) and CD11b (1:500, Invitrogen PA5-79532), using a Pierce peroxidase IHC detection kit (Thermo Fisher). QuPath V0.3.2 software was used for automated analysis using surface and cytoplasmic staining to determine the percent cells positive for CD11b. The staining scores for pSTAT3 were evaluated by the pathologist and calculated by (% of positively stained tumor cells x staining intensity ranged from 0-3). Values from at least two cores from each sample were considered valid and an average score was taken.

siRNA Knockdown, RNA Extraction, and qPCR

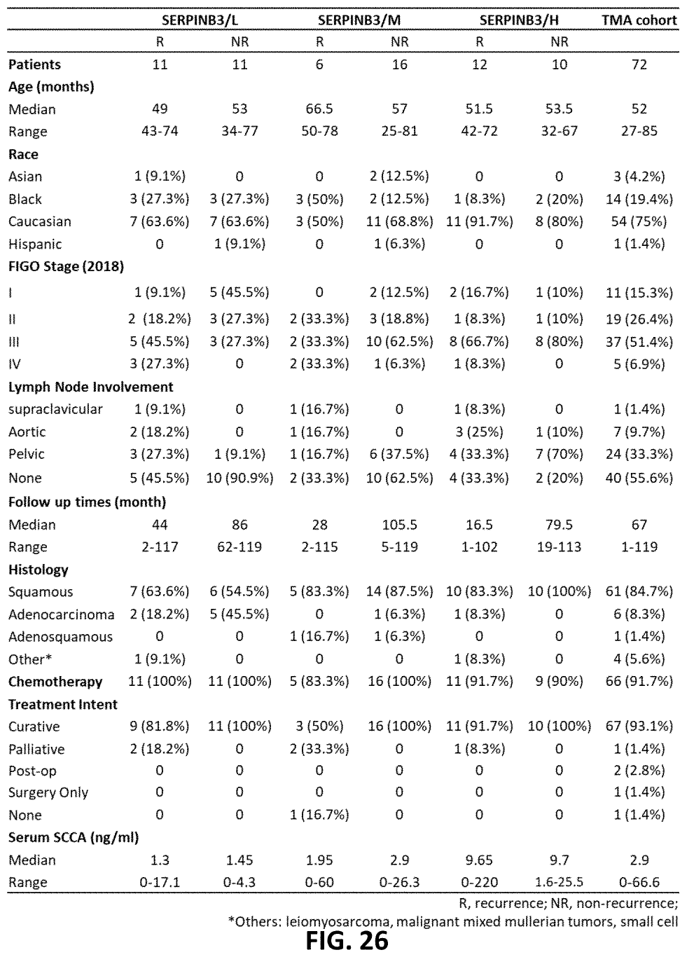

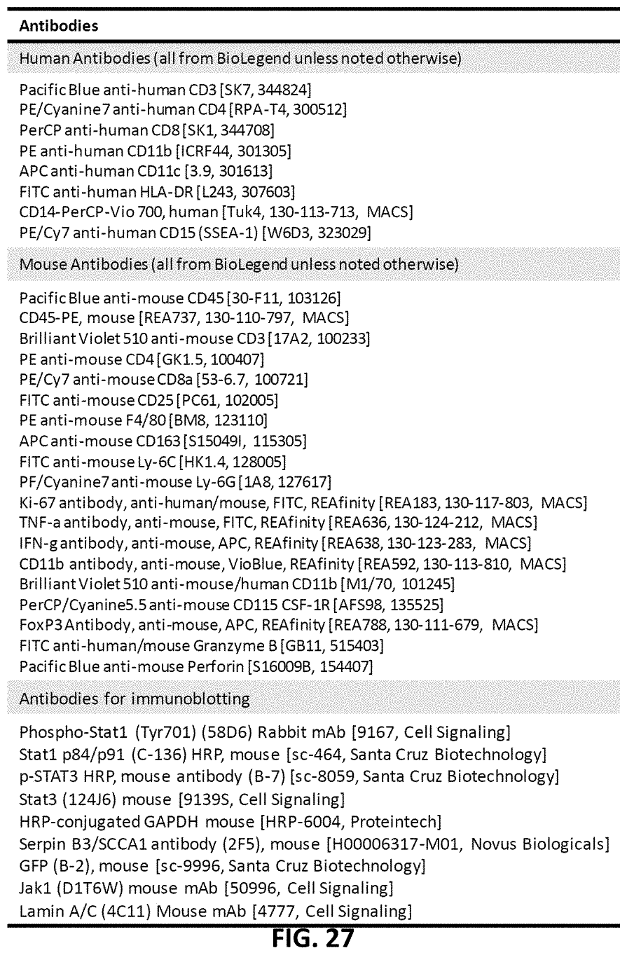

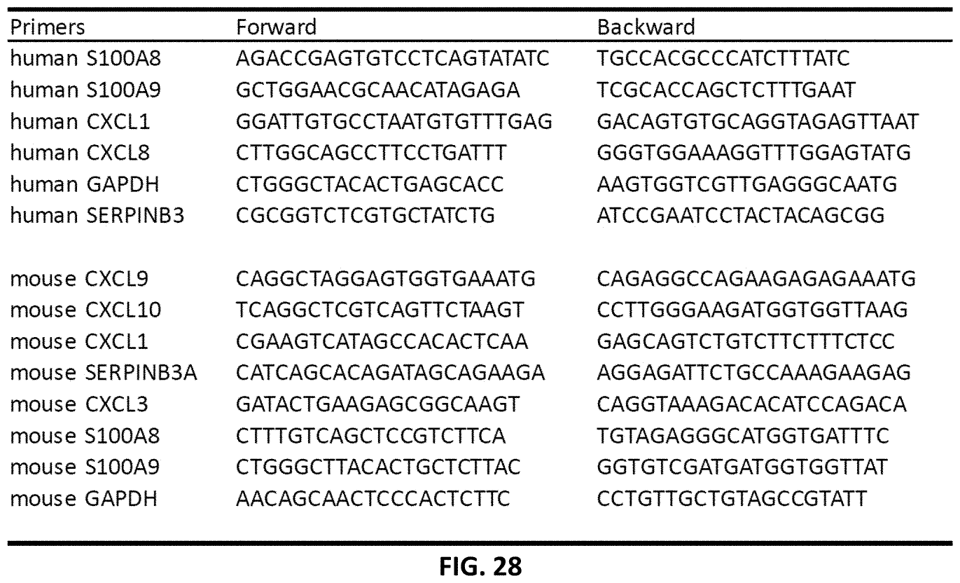

Lipofectamine RNAiMAX (Thermo Fisher) was used for STAT1 siRNA (ID: SASI_Hs02_00343387, SASI_Hs01_00098937, Sigma), STAT3 siRNA (ID: SASI_Hs01_00121206, SASI_Hs01_00061860, Sigma), and negative control siRNA (SIC001, Sigma) transfection, according to the manufacturer's instructions. RNA was isolated using GenElute Mammalian Total RNA Miniprep Kit (Sigma) and reverse transcribed to cDNA using a High Capacity cDNA Reverse Transcription Kit (Thermo Fisher). Quantitative polymerase chain reaction (qPCR) was performed using PowerUp SYBR green PCR Master Mix (Applied Biosystems) and the Applied Biosystems 7900 Fast real-time PCR system and software. Each sample was performed in triplicate, gene expression levels were normalized to GAPDH, and fold changes were calculated using the ΔΔCt method. Sequences of primers are detailed in FIG. 28 and Table 1 below.

TABLE 1
qPCR Primers

| | | | | |
|---|---|---|---|---|
| | SEQ | | SEQ | |
| | ID | | ID | |
| Primers | NO: | Forward | NO: | Backward |

Human

| | | | | |
|---|---|---|---|---|
| S100A8 | 1 | AGACCGAGTGTCCT | 2 | TGCCACGCCCATCT |
| | | CAGTATATC | | TTATC |
| S100A9 | 3 | GCTGGAACGCAACA | 4 | TCGCACCAGCTCTT |
| | | TAGAGA | | TGAAT |
| CXCL1 | 5 | GGATTGTGCCTAAT | 6 | GACAGTGTGCAGG |
| | | GTGTTTGAG | | TAGAGTTAAT |
| CXCL8 | 7 | CTTGGCAGCCTTCC | 8 | GGGTGGAAAGGTT |
| | | TGATTT | | TGGAGTATG |
| GAPDH | 9 | CTGGGCTACACTGA | 10 | AAGTGGTCGTTGAG |
| | | GCACC | | GGCAATG |
| SERPINB3 | 11 | CGCGGTCTCGTGCT | 12 | ATCCGAATCCTACT |
| | | ATCTG | | ACAGCGG |

Mouse

| | | | | |
|---|---|---|---|---|
| CXCL9 | 13 | CAGGCTAGGAGTGG | 14 | CAGAGGCCAGAAG |
| | | TGAAATG | | AGAGAAATG |
| CXCL10 | 15 | TCAGGCTCGTCAGT | 16 | CCTTGGGAAGATG |
| | | TCTAAGT | | GTGGTTAAG |
| CXCL1 | 17 | CGAAGTCATAGCCA | 18 | GAGCAGTCTGTCTT |
| | | CACTCAA | | CTTTCTCC |
| SERPINB3A | 19 | CATCAGCACAGATA | 20 | AGGAGATTCTGCCA |
| | | GCAGAAGA | | AAGAAGAG |
| CXCL3 | 21 | GATACTGAAGAGCG | 22 | CAGGTAAAGACACA |
| | | GCAAGT | | TCCAGACA |
| S100A8 | 23 | CTTTGTCAGCTCCGT | 24 | TGTAGAGGGCATG |
| | | CTTCA | | GTGATTTC |
| S100A9 | 25 | CTGGGCTTACACTG | 26 | GGTGTCGATGATG |
| | | CTCTTAC | | GTGGTTAT |
| GAPDH | 27 | AACAGCAACTCCCA | 28 | CCTGTTGCTGTAGC |
| | | CTCTTC | | CGTATT |

### Enzyme-Linked Immunosorbent Assay (ELISA)

Cell culture supernatant was collected at 48 h after fresh media was added to the adherent cells in a monolayer. Cell lysates were prepared using NP-40 buffer (Alfa Aesar). Quantification of human/mouse chemokines in tissue culture supernatants and tissue homogenates was performed using a commercially available human CXCL1/GRO alpha, human IL-8/CXCL8, human S100A8/S100A9 Heterodimer, mouse CXCL1/KC, and mouse S100A8/S100A9 HeterodimerDuoSet ELISA kit from R&D Systems. Mouse GRO gamma ELISA Kit was obtained from Abcam. Chemokine concentration in samples was determined by interpolation from a standard curve.

### Phosphorylation Protein Array

Human Phosphorylation Pathway Profiling Array C55 consisted of the detection of 55 phosphorylated proteins (RayBiotech). The same amount of protein from each sample was used for screening and assays were performed according to the manufacturer's instruction. Array blots were scanned with the Bio-Rad ChemiDoc MP imaging system and images were processed using the Protein Array Analyzer plug-in of the ImageJ program.

### Co-Immunoprecipitation and Immunoblotting

Immunoprecipitation of Jak1 was performed using a Pierce co-immunoprecipitation kit (Thermo Fisher). Cell fractionation was carried out using NE-PER nuclear and cytoplasmic extraction reagents kit (Thermo Fisher). The purity of non-nuclear and nuclear fractions was determined using GAPDH and lamin A/C, respectively. For immunoblotting, cells were lysed with RIPA buffer (Cell Signaling) supplemented with proteinase/phosphatase inhibitors (Thermo Fisher). Protein concentrations were determined using BCA (Thermo Fisher) and proteins were electrophoresed on 4-20% gradient gels (Bio-Rad), transferred to PVDF blots using the Trans-Blot TurboTransfer system (Bio-Rad), and incubated with antibodies shown in FIG. 27. Chemiluminescence was detected by using an ECL reagent (Cytiva) and visualized using the Bio-Rad ChemiDoc MP imaging system and Image Lab software (Bio-Rad).

### Statistics

Statistical analyses were performed using GraphPad Prism version 8 and all values are reported as mean±SEM. Two-tailed unpaired t-test was used for two groups comparisons and one-way or two-way ANOVA was used for multiple comparisons. P values of less than 0.05 were considered statistically significant (*P<0.05; **P<0.01; ***P<0.001).

What is claimed is:

1. A method for treating a squamous cell carcinoma in a patient in need, comprising administering a therapeutically effective amount of an active agent configured to modulate STAT-related immunosuppression as an adjuvant to a radiotherapy treatment.

2. The method of claim 1, wherein the active agent comprises a STAT modulator or a SERPINB3 inhibiting agent.

3. The method of claim 2, wherein the STAT modulator is selected from an anti-STAT antibody, a fusion protein, a small molecule, an inhibitory protein, and an sgRNA targeting a STAT

4. The method of claim 3, wherein the STAT modulator is a STAT3 inhibitor.

5. The method of claim 2, wherein the SERPINB3 inhibiting agent is selected from an anti-SERPINB3 antibody, a fusion protein, a small molecule, an inhibitory protein, and an sgRNA targeting SERPINB3.

6. The method of claim 1, wherein the active agent is Ruxolitinib.

7. A method of selecting a treatment for a patient with a squamous cell carcinoma, the method comprising:
a. detecting an expression level of SIRPINB3 of the patient;
b. selecting a treatment comprising radiotherapy and administration of a therapeutically effective amount of an active agent if the expression level of SIRPINB3 falls above a threshold level.

8. The method of claim 7, wherein detecting the expression level of SIRPINB3 within the tumor comprises obtaining a serum concentration of SCCA from the patient.

9. The method of claim 8, wherein the threshold level is a serum SCCA concentration of about 9.16 ng/ml.

10. The method of claim 9, wherein the threshold level is a serum SCCA concentration of about 16.1 ng/ml.

11. The method of claim 7, further comprising detecting a pSTAT3 score within a sample of tumor cells stained for pSTAT3 and selecting a treatment comprising radiotherapy and administration of a therapeutically effective amount of the active agent if the expression level of SIRPINB3 falls above a threshold level and the pSTAT3 score falls above a second threshold level.

12. The method of claim 11, wherein the pSTAT score comprises a percentage of positively-stained tumor cells weighted by an intensity score, wherein the intensity score ranges from about 1 to about 3.

13. The method of claim 12, wherein the second threshold comprises a pSTAT score of about 100.

14. The method of claim 7, wherein the active agent is configured to modulate STAT-related immunosuppression as an adjuvant to a radiotherapy treatment.

15. The method of claim 14, wherein the active agent comprises a STAT modulator or a SERPINB3 inhibiting agent.

16. The method of claim 15, wherein the STAT modulator is selected from an anti-STAT antibody, a fusion protein, a small molecule, an inhibitory protein, and an sgRNA targeting STAT.

17. The method of claim 16, wherein the STAT modulator is a STAT3 inhibitor.

18. The method of claim 14, wherein the SERPINB3 inhibiting agent is selected from an anti-SERPINB3 antibody, a fusion protein, a small molecule, an inhibitory protein, and an sgRNA targeting SERPINB3.

19. The method of claim 7, wherein the active agent is Ruxolitinib.


SEQUENCE LISTING (28 sequences)

SEQ ID NO: 1 - DNA, 23 bp, synthetic construct
  source 1..23: mol_type = other DNA
agaccgagtgtcctcagtatatc

SEQ ID NO: 2 - DNA, 19 bp, synthetic construct
  source 1..19: mol_type = other DNA
tgccacgcccatctttatc

SEQ ID NO: 3 - DNA, 20 bp, synthetic construct
  source 1..20: mol_type = other DNA
gctggaacgcaacatagaga

SEQ ID NO: 4 - DNA, 19 bp, synthetic construct
  source 1..19: mol_type = other DNA
tcgcaccagctctttgaat

SEQ ID NO: 5 - DNA, 23 bp, synthetic construct
  source 1..23: mol_type = other DNA
ggattgtgcctaatgtgtttgag

SEQ ID NO: 6 - DNA, 23 bp, synthetic construct
  source 1..23: mol_type = other DNA
gacagtgtgcaggtagagttaat

SEQ ID NO: 7 - DNA, 20 bp, synthetic construct
  source 1..20: mol_type = other DNA
cttggcagccttcctgattt

SEQ ID NO: 8 - DNA, 22 bp, synthetic construct
  source 1..22: mol_type = other DNA
gggtggaaaggtttggagtatg

SEQ ID NO: 9 - DNA, 19 bp, synthetic construct
  source 1..19: mol_type = other DNA
ctgggctacactgagcacc

SEQ ID NO: 10 - DNA, 21 bp, synthetic construct
  source 1..21: mol_type = other DNA
aagtggtcgttgagggcaatg

SEQ ID NO: 11 - DNA, 19 bp, synthetic construct
  source 1..19: mol_type = other DNA
cgcggtctcgtgctatctg

SEQ ID NO: 12 - DNA, 21 bp, synthetic construct
  source 1..21: mol_type = other DNA
atccgaatcctactacagcgg

SEQ ID NO: 13 - DNA, 21 bp, synthetic construct
  source 1..21: mol_type = other DNA
caggctaggagtggtgaaatg

SEQ ID NO: 14 - DNA, 22 bp, synthetic construct
  source 1..22: mol_type = other DNA
cagaggccagaagagagaaatg

SEQ ID NO: 15 - DNA, 21 bp, synthetic construct
  source 1..21: mol_type = other DNA
tcaggctcgtcagttctaagt

SEQ ID NO: 16 - DNA, 22 bp, synthetic construct
  source 1..22: mol_type = other DNA
ccttgggaagatggtggttaag

SEQ ID NO: 17 - DNA, 21 bp, synthetic construct
  source 1..21: mol_type = other DNA
cgaagtcatagccacactcaa

SEQ ID NO: 18 - DNA, 22 bp, synthetic construct
  source 1..22: mol_type = other DNA
gagcagtctgtcttctttctcc

SEQ ID NO: 19 - DNA, 22 bp, synthetic construct
  source 1..22: mol_type = other DNA
catcagcacagatagcagaaga

SEQ ID NO: 20 - DNA, 22 bp, synthetic construct
  source 1..22: mol_type = other DNA
aggagattctgccaaagaagag

SEQ ID NO: 21 - DNA, 20 bp, synthetic construct
  source 1..20: mol_type = other DNA
gatactgaagagcggcaagt

SEQ ID NO: 22 - DNA, 22 bp, synthetic construct
  source 1..22: mol_type = other DNA
caggtaaagacacatccagaca

SEQ ID NO: 23 - DNA, 20 bp, synthetic construct
  source 1..20: mol_type = other DNA
ctttgtcagctccgtcttca

SEQ ID NO: 24 - DNA, 21 bp, synthetic construct
  source 1..21: mol_type = other DNA
tgtagagggcatggtgatttc

SEQ ID NO: 25 - DNA, 21 bp, synthetic construct
  source 1..21: mol_type = other DNA
ctgggcttacactgctcttac

SEQ ID NO: 26 - DNA, 21 bp, synthetic construct
  source 1..21: mol_type = other DNA
ggtgtcgatgatggtggttat

SEQ ID NO: 27 - DNA, 20 bp, synthetic construct
  source 1..20: mol_type = other DNA
aacagcaactcccactcttc

SEQ ID NO: 28 - DNA, 20 bp, synthetic construct
  source 1..20: mol_type = other DNA
cctgttgctgtagccgtatt